(19) United States
(12) Patent Application Publication
(10) Pub. No.: US 2023/0227538 A1
(43) Pub. Date: Jul. 20, 2023

(54) SARS-COV2 NEUTRALIZING SINGLE DOMAIN ANTIBODY CONSTRUCTS

(71) Applicant: REGENTS OF THE UNIVERSITY OF CALIFORNIA, Oakland, CA (US)

(72) Inventors: Peter Walter, Oakland, CA (US); Aashish Manglik, Oakland, CA (US); Michael SCHOOF, Oakland, CA (US); Bryan FAUST, Oakland, CA (US); Nicholas HOPPE, Oakland, CA (US); Reuben SAUNDERS, Oakland, CA (US); Christian BILLESBOELLE, Oakland, CA (US)

(21) Appl. No.: 17/998,605

(22) PCT Filed: May 12, 2021
(86) PCT No.: PCT/US21/32092

§ 371 Date: Nov. 11, 2022

Related U.S. Application Data
(60) Provisional application No. 63/023,645, filed on May 12, 2020.
(60) Provisional application No. 63/042,135, filed on Jun. 22, 2020.
(60) Provisional application No. 63/047,655, filed on Jul. 2, 2020.
(60) Provisional application No. 63/061,928, filed on Aug. 6, 2020.

(51) Int. Cl. C07K 16/10; A61P 31/14

(52) U.S. Cl. CPC C07K 16/10; A61P 31/14; A61K 2039/505

(57) ABSTRACT

Antibodies, including single-domain antibodies, that bind to SARS-CoV2 virus and methods of treatment using single-do-main antibodies that bind to SARS-CoV2 virus are provided.

### I. BACKGROUND OF THE INVENTION

Severe acute respiratory syndrome coronavirus 2 or “SARS-CoV-2” is a virus strain that causes coronavirus disease 2019 (COVID-19). See, e.g., Gorbalenya A E, et al. Nature Microbiology. 5 (4): 536-544 (March 2020). Therapeutic treatments to address the global pandemic are needed.

### II. BRIEF DESCRIPTION OF THE DRAWINGS

FIGS. 1A and 1B depicts the general strategy for blocking the entry of the SARS-CoV2 virus (“SC2 virus”). As generally discussed herein, the spike protein of the SC2 virus forms a trimeric structure that binds to the extracellular domain of the ACE2 receptor on human cells at a location deemed the spike receptor binding domain (RBD). FIG. 1A depicts a space filling model and FIG. 1B uses a ribbon diagram. FIG. 1B shows that by blocking the ACE2—spike protein interaction, the SC2 virus can no longer enter the host cells.

FIGS. 2A, 2B and 2C show the validation of the correct structure for the spike trimeric antigen comprising residues extracellular domain (ECD) residues 1-1208, stabilizing mutations P986 and P987, a substitution for the furin cleavage site and a C-terminal trimerization motif (hereafter termed “spike ECD”). FIG. 2A shows a model of the structure of the SC2 spike ECD binding the human ACE2 receptor, showing the location of the RBD within the spike ECD. In contrast to other studies, the spike ECD was used to generate antigen binding domains (ABDs) in the present invention. FIG. 2B shows that using Cryogenic Electron Microscopy (“cryo-EM”) the correct trimeric spike protein ECD structure of the antigen was used herein. FIG. 2C depicts the antigen validation using a spike ECD-ACE2 binding assay, showing a K_{D} of 44 nM, a kₐ of 32.6×10⁵ M⁻¹s⁻¹ and a k_{d} of 0.012 s⁻¹.

FIG. 3 depicts the binding of a candidate MASC protein, “AeroNab6”, to SC2 spike ECD, that competes for binding to that is competitive with ACE2. MASC protein (monomer) was displayed on the surface of yeast by fusion to a HA-epitope tagged “stalk” protein that tethers the MASC protein to the yeast cell surface. Yeast displaying the MASC protein were incubated for 30 minutes at room temperature with 1 nM purified spike ECD labeled with Alexa 647 fluorophore (Spike-Alexa 647) and 10 μg/mL anti-HA Alexa488 antibody (12CA5) in assay buffer (20 mM HEPES pH 8.0, 150 mM sodium chloride, and 0.1% bovine serum albumin). Yeast were subsequently washed with assay buffer to remove unbound spike ECD and amount of spike ECD binding on the yeast surface was assessed by flow cytometry. Spike ECD binding was indicated by simultaneous presence of Alexa 647 and Alexa 488 fluorescence. To assess competition with ACE2, the assay above was repeated with 1.4 μM ACE2-Fc (a fusion protein comprising the ACE2 ECD to the human IgG1 Fc domain). A decrease in Alexa 647 fluorescence, corresponding to a loss of spike ECD binding, indicates that the MASC protein binds to spike ECD at an epitope that is competitive with ACE2.

FIGS. 4A and 4B depicts a schematic of the “up” and “down” conformations of the RBD domains of spike protein trimers. FIG. 4A shows a cryo-EM structure of the “down” or “off” position on the left and engaged with the ACE2 receptor in the “extended” or “on” position on the right. The RBD must be extended in order to engage the ACE2 receptor. FIG. 4B shows a cryo-EM structure at ˜3.0 Å resolution with a MASC protein monomer, AeroNab6, showing that the AeroNab6 MASC monomer binds to the “down” conformation of the Spike trimer on the left, thus preventing the binding of ACE2. On the right of FIG. 4B is a top view of the structure, showing three AeroNab6 monomers engaged on the Spike trimer. Additionally, this structure shows a minimal linker-length for a multimeric form of the AeroNab6 MASC protein that simultaneously binds more than one RBD. The distance between the N- and C-termini of individual AeroNab6 monomers bound to spike ECD in the “down” state is 51 Å. This requires ≥15 amino acids to bridge individual subunits to simultaneously engage multiple RBD monomers.

FIGS. 5A and 5B depict the mechanism of action of the MASC proteins of the invention. FIG. 5A shows that the AeroNab6 MASC protein engages with one RBD of the trimer using CDR1 and CDR2, and a second RBD of the trimer with CDR3. This is extremely effective in locking the RBD into the “off” position with extremely high affinity, as discussed further below. This structure also identifies the contact residues to facilitate affinity maturation. As further discussed below, AeroNab6 MASC makes extensive contacts within the ACE2 binding region of the SC2 spike RBD, including residues 446, 447, 449, 453, 455, 456, 483-486, 489-490, 493-496, 498, 501, and 505). The CDR3 of AeroNab6 MASC contacts a neighboring RBD on the SC2 spike at a three-dimensional epitope defined by residues 342, 343, 367, 371-375, 404, 436-441. This additional contact enables AeroNab6 MASC to locking the neighboring RBD in the “off” position, while simultaneously disrupting ACE2 binding at an adjacent RBD. FIG. 5B shows the overlap between the binding site of the AeroNab6 MASC molecule to the Spike protein with the binding site of ACE2 to the Spike protein. This overlap explains the fact that the AeroNab6 MASC monomer still blocks ACE2 from binding to monomeric RBDs.

FIGS. 6A, 6B and 6C depict the increase in potency as higher valency MASC proteins are made. FIG. 6A shows the monomeric AeroNab6 binding kinetics, with a KD of 210 nM. FIG. 6B shows the increase in binding affinity of the dimeric MASC fusion protein, and FIG. 6C shows the further increase of a trimeric MASC fusion protein. Further decrease in the dissociation kinetics between the dimeric and trimeric fusion proteins suggests engagement of all three SC2 spike RBDs by the trimeric MASC.

FIGS. 7A and 7B show affinity maturation of one MASC monomer, AeroNab6. FIG. 7A shows mutations were made in vhhCDR1 and vhhCDR2, which binds to a first RBD, and in vhhCDR3, which binds to a second RBD of the Spike trimer. FIG. 7B shows the binding kinectics of the parent protein, AeroNab6, and one of the affinity matured candidates, AeroNab6ₘ, as measured by surface plasmon resonance (SPR). For this particular antigen binding domain, there was a 500-fold enhancement of binding to the Spike protein.

FIGS. 8A, 8B and 8C show the increase in binding affinity of an affinity matured MASC protein candidate, AeroNab6m X 3. FIG. 8A is the parental AeroNab6, AeroNab6ₘ is an affinity matured protein and AeroNab6ₘX3 is the trimeric form, designed to bind to the trimeric Spike protein, as measured by SPR. Surprisingly and fortuitously, the trimeric AeroNab6ₘX3 disassociates from the Spike protein with a half-life of at least weeks. The theoretical dissociation half-life for AeroNab6ₘX3 predicted by the dissociation kinetics of the monomer and simultaneous engagement of three RBDs is >100 years.

FIGS. 9A, 9B and 9C depicts the successful humanization of the AeroNab6 MASC protein. FIG. 9A shows the starting kinetic parameters of the AeroNab6, with the llama framework regions shown in FIG. 9B. The CDRs are transplanted onto a human heavy chain framework (IGHV3-66) as shown in FIG. 9C. The humanized version, AeroNabh, has only two amino acid substitutions in the human IGHV3-66 sequence as shown in FIG. 17. As shown in FIG. 9D, the humanization substitutions do not cause significant loss of affinity for the Spike protein.

FIG. 10 depicts a pseudovirus neutralization assay, using infection of human ACE2-expressing HEK293 cells with a lentiviral construct containing the SARS-CoV2 Spike protein. As shown in the Figure, the trimeric MASC fusion proteins show higher neutralization than the MASC monomers. Additionally, the affinity matured MASC proteins show increased potency as well.

FIG. 11 shows a real viral neutralization assay, measuring inhibition of SARS-CoV2 infection of VeroE6 cells by the MASC test articles shown, with viral quantification after 72 hours. As shown, the trimeric MASC fusion proteins show higher neutralization than the MASC monomers. Additionally, the affinity matured MASC proteins show increased potency as well. Neutralization of authentic SARS-CoV2 was performed using a plaque reduction neutralization test. MASC proteins were serially diluted in culture medium and mixed with 100 μL of 500 TCID50 SARS-CoV2 for 1 hour. The mixture was added to VeroE6 cells and incubated for 1 hour, after which the cells were overlaid with a solid support to allow the development of plaques, which were quantified on day 3. The half maximal inhibitory concentrations (IC50) were determined using 3-parameter logistic regression.

FIG. 12 shows a table summarizing some of the data with a set of the MASC proteins indicated.

FIG. 13 depicts the sequences of some sdABDs in the original screening, including the CDRs and each framework, noting that FR2 in some of the original clones was also changed.

FIG. 14 depicts the full length sequences of the sdABDs of the MASC proteins corresponding to the clones in FIG. 13.

FIG. 15 depicts the framework backbone and the CDR sets for a number of different MASC protein of the invention.

FIG. 16 depicts the sdABD sequences of a number of MASC monomers based on the CDRs disclosed herein.

FIGS. 17A and 17B depicts some sequences of use in the invention. FIG. 17A depicts the sequence of the spike antigen used in the generation of the data herein and FIG. 17B is the sequence of the human ACE2 extracellular domain (ECD). The SC2 Spike ECD used for MASC protein identification used a construct encoding residues 1-1208 of SARS-Cov2 with proline substitutions at 986/987 and a substitution for the furin cleavage site (GSAS for residues 682-685). A C-terminal T4 fibritin trimerization motif was included, followed by a rhinovirus 3C protease cleavage site, an 8× histidine tag, and a Twin Strep Tag (as described in Wrapp et al Science 2020). The SC2 Spike ECD construct was expressed in either Expi293 or ExpiCHO cells (Thermo) per manufacturer instructions. SC2 Spike ECD was purified by a combination of metal affinity and size exclusion chromatography.

FIG. 18 depicts some sequences of particular use in the present invention. The CDRs are each underlined, and the junctions between the sdABDs and the linkers are shown as slashes (“/”).

FIG. 19 depicts the significant lyophilization stability of a trimeric MASC fusion protein, AeroNab6X3. Pre- and post-lyophilization measurements by either superdex S200 gel filtration or SPR analysis on the immobilized Spike protein show that the MASC fusion proteins of the invention can be lyophilized with no aggregation, denaturation or activity loss, in that binding is preserved.

FIGS. 20A, 20B and 20C shows the significant stability to aerosolization by a trimeric MASC fusion protein, AeroNab6X3. FIG. 18A shows an inexpensive nebulizer that creates 3.5 μm droplets. Using Superdex S200 gel filtration, the result show that the fusion protein is stable to aerosolization with no aggregation or denaturation comparing pre-aerosolization (FIG. 18B) and post-aerosolization (FIG. 18C).

FIG. 21 shows the significant increase in affinity achieved in Example 2. Yeast displaying nanobody variants of NbCOV6 were incubated with fluorescent SARS-Cov2 Spike receptor binding domain (RBD). The amount of RBD bound to the yeast cell surface was quantified by flow cytometry. The pool of affinity matured variants titrate with increased potency compared to the parent NbCOV6, indicative of higher affinity to the receptor binding domain.

FIG. 22 shows a comparison of the SPR affinities for the original parental anti-Spike MASC proteins measured using immobilized SC2 spike ECD.

FIG. 23 shows comparison of the SPR affinities for a number of MASC proteins and fusion proteins measured using immobilized SC2 spike ECD.

FIG. 24 shows the humanization strategy for AeroNab6, showing the close similarity of the parental clone for human IGHV3-66 sequence.

FIG. 25 shows useful CDR sets and the framework regions of the invention.

FIG. 26 shows the sequences of two dimeric MASC constructs using the AeroNab6mh sdABD and the NbCOV003 sdABD.

FIG. 27 shows data to support Example 4.

FIG. 28 shows data to support Example 4.

FIG. 29 shows data to support Example 4.

FIG. 30 shows data to support Example 4.

FIG. 31 shows data to support Example 4.

FIG. 32 shows the Cryo-EM workflow for Nb6. A flowchart representation of the classification workflow for Spike^{S2P}-Nb6 complexes yielding open and closed Spike^{S2P} conformations. From top to bottom, particles were template picked with a set of 20 Å low-pass filtered 2D backprojections of apo-Spike^{S2P} in the closed conformation. Extracted particles in 2D classes suggestive of various Spike^{S2P} views were subject to a round of heterogenous refinement in cryoSPARC with two naïve classes generated from a truncated Ab initio job, and a 20 Å low-pass filtered volume of apo-Spike^{S2P} in the closed conformation. Particles in the Spike^{S2P} 3D class were subject to 25 iterations of 3D classification into 6 classes without alignment in RELION, using the same input volume from cryoSPARC 3D classification, low pass filtered to 60 Å, T=8. Particles in classes representing the open and closed Spike^{S2P} conformations were imported into cisTEM for automatic refinement. Viewing distribution plots were generated with pyEM, and visualized with ChimeraX. Half maps from refinement were imported into cryoSPARC for local resolution estimation as shown in FIG. S3.

FIG. 33 shows the Cryo-EM workflow for Nb11. Classification workflow for Spike^{S2P}-Nb11 complexes yielding open and closed Spike^{S2P} conformations. Particles were template picked from two separate collections with a set of 20 Å low-pass filtered 2D backprojections of apo-Spike^{S2P} in the closed conformation. Extracted particles were Fourier cropped to 128 pixels prior to extensive heterogenous refinement in cryoSPARC, using a 20 Å low-pass filtered volume of apo-Spike^{S2P} in the closed conformation and additional naïve classes for removal of non-Spike^{S2P} particles. After cryoSPARC micrograph curation and heterogenous refinement, Spike^{S2P} density corresponding to all regions outside of the ACE2 RBD::Nanobody interface were subtracted. A mask around the ACE2 RBD::Nanobody interface was generated, and used for multiple rounds of 3D classification without alignment in RELION. Particles in classes representing open and closed Spike^{S2P} conformations were selected, unsubtracted and unbinned prior to refinement in RELION. Viewing distribution plots were generated with pyEM, and visualized with ChimeraX. Half maps from refinement were imported into cryoSPARC for local resolution estimation as shown in FIG. S3.

FIG. 34 shows resolution of cryo-EM maps and models. A. Local resolution estimates of Spike^{S2P} complexes as generated in cryoSPARC. All maps (except mNb6) are shown with the same enclosed volume. All maps are colored on the same scale, as indicated. B. Gold standard Fourier Shell Correlation (GSFSC) plots for cryo-EM maps calculated within cryoSPARC. Resolution values in parentheses represent values at FSC=0.143 (dashed line). C. Model-map correlation calculated in Phenix. Resolution values in parentheses represent values at FSC=0.5 (dashed line).

FIG. 35 shows modeling of distances for multimeric nanobody design. A. Model of Spike^{S2P}:Nb6 complex in the closed state. The minimal distance between adjacent Nb6 N- and C-termini is 52 Å (dashed line). B. Model of Spike^{S2P}:Nb6 complex in the open state with Nb6 docked into the cryo-EM density for up-state RBD. Minimal distance between N- and C-termini of both nanobodies is 72 Å. Nb6 cannot bind RBD2 in open Spike^{S2P}, as this would sterically clash with RBD3. C. Model of Spike^{S2P}:Nb11 complex in the closed state. The minimal distance between adjacent Nb6 N- and C-termini is 71 Å (dashed line). D. of Spike^{S2P}:Nb11 complex in the open state. The minimal distance between adjacent Nb6 N- and C-termini is 68 Å between Nb 11 bound to RBD2 in the down-state and RBD3 in the up-state. For B, the model of Nb6 from A was docked into the cryo-EM map to enable modeling of distance between N- and C-termini. For C and D, a generic nanobody was docked into cryo-EM maps to model the distance between N- and C-termini.

FIG. 36 shows radiolytic hydroxyl radical footprinting of Spike^{S2P}. A. Change in oxidation rate between Spike^{S2P} and Nb3-Spike^{S2P} complexes at all residues. A cluster of highly protected residues in the Spike^{S2P}-Nb3 complex is observed in the N-terminal domain. B. Oxidation rate plots of the two (M177, H207) most heavily protected residues upon Nb3 binding to Spike^{S2P}. Data points labeled with an asterisk are excluded from rate calculations as these values fall outside of the first order reaction, likely due to extensive oxidation-mediated damage. C. Change in oxidation rate mapped onto Spike in the all RBD down conformation.

FIG. 37 shows multivalent Nb3 construct inhibits Spike^{S2P}:ACE2 interaction. A. SPR experiments with immobilized Spike^{S2P} show that Nb3 and monovalent ACE2 can bind Spike^{S2P} simultaneously. The order of Nb3 and monovalent ACE2 does not affect the binding of the second reagent. Nb3 therefore does not inhibit Spike^{S2P} binding to monovalent ACE2. B. Nanobody inhibition of 1 nM Spike^{S2P}-Alexa 647 binding to ACE2 expressing HEK293T cells by either monovalent or trivalent Nb3. n=2 biological replicates for Nb3-tri. All error bars represent s.e.m.

FIG. 38 shows CryoEM workflow for mNb6. Classification workflow for the Spike^{S2P}-mNb6 complex yielding a closed Spike^{S2P} conformation. From top to bottom, particles were template picked from two separate collections with a set of 20 Å low-pass filtered 2D backprojections of apo-Spike^{S2P} in the closed conformation. Extracted particles were Fourier cropped to 96 pixels prior to 2D classification. Particles in Spike^{S2P} 2D classes were selected for a round of heterogeneous refinement in cryoSPARC using a 20 Å low-pass filtered volume of apo-Spike^{S2P} in the closed conformation and additional naïve classes for removal of non-Spike^{S2P} particles. In RELION, particles in the Spike^{S2P} 3D class were subject to two rounds of 3D classification without alignment into 6 classes using the same input volume from cryoSPARC 3D classification, low pass filtered to 60 Å, T=8. Unbinned particles in the Spike^{S2P}-closed conformation were exported into cisTEM for automatic refinement, followed by local refinement using a mask around the RBD::Nanobody interface. Viewing distribution plots were generated with pyEM, and visualized with ChimeraX. Half maps from refinement were imported into cryoSPARC for local resolution estimation as shown in FIG. S3.

FIG. 39 shows mNb6 and Nb3-tri are additive for viral neutralization. Inhibition of pseudotyped lentivirus infection of ACE2 expressing HEK293T cells by mNb6 with increasing concentrations of Nb3-tri. mNb6 neutralization is additive with Nb3-tri, as demonstrated by inhibitory activity at a sub-saturating dose of Nb3-tri. However, the potency of mNb6 is unchanged by Nb3-tri, suggesting no synergistic effect on viral neutralization.

FIG. 40 shows stability of Nb6 and its derivatives. A. Thermal denaturation of nanobodies assessed by circular dichroism measurement of molar ellipticity at 204 nm. Apparent melting temperatures (Tₘ) for each nanobody are indicated. B. Nanobody inhibition of 1 nM Spike^{S2P}-Alexa 647 binding to ACE2 expressing HEK293T cells after incubation at either 25° C. or 50° C. for 1 hour or after aerosolization. C. Inhibition of pseudotyped lentivirus infection of ACE2 expressing HEK293T cells by mNb6-tri after aerosolization, lyophilization, or heat treatment at 50° C. for 1 hour.

FIG. 41 shows nanobody affinities and efficacies in neutralization assays. aAverage values from n=5 biological replicates for Nb6, Nb11, Nb15, Nb19 are presented, all others were tested with n=3 biological replicates.bAverage values from n=2 biological replicates for Nb12, Nb17, and Nb11-tri are presented, all others were tested with n=3 biological replicates. cAverage values from n=2 biological replicates for Nb3, Nb3-bi, and Nb3-tri. n=3 biological replicates for all others. dNb3, Nb17, and Nb18 expresses at 41.3, 4.0, and 2.2 milligrams per liter of E. coli culture, respectively. Nb3 is monodisperse on size exclusion chromatography over a GE S200 Increase 10×300 column, while Nb17 and Nb18 are polydisperse. NB—no binding. NC—no competition. NP—not performed.

FIG. 42 shows Cryo-electron microscopy data collection and refinement statistics.

FIG. 43 shows X-ray crystallography data collection and refinement statistics.

FIG. 44 shows X-ray crystallography data collection and refinement statistics. ^{a} Values in parentheses correspond to the highest resolution shell. ^{b} R_{merge}=Σ|I−<I>I/ΣI.

FIG. 45 shows nanobody expression plasmids.

FIG. 46 shows Biophysical stability of AeroNab6mhx3. AeroNab6mhx3 is resistant to thermal denaturation. Circular dichroism of AeroNabs measured over increasing temperatures shows loss of beta-sheet character at 204 nm. Melting temperatures (Tm) were calculated as loss of 50% signal.

FIG. 47 shows the structure of Spike bound to mNb6. Cryo-EM structure of mNb6 bound to Spike shows stabilization of closed Spike conformation.

FIG. 48 shows mNb6 X-Ray Structure (apo- and Spike-bound). CDR1 and CDR3 bind by an adaptive fit mechanism.

FIG. 49 shows other nanobodies from primary screen.

FIG. 50 shows AeroNab3 targets an allosteric epitope. Inhibition of SARS-CoV2 infection of VeroE6 cells by indicated dose of AeroNab constructs. Viral plaques were quantified after 72 hours. AeroNab3 targets a unique epitope on Spike to neutralize viral infection.

FIG. 51 shows the Experimental Design of a Transmission Study for Example 5.

FIG. 52 shows the Experimental Design of an Efficacy Study for Example 5.

FIG. 53 shows lung virus titers of golden Syrian hamsters after treatment with Nanoparticle A prior to cohabitation with SARS-CoV-2-infected animals as described in Example 5.

FIG. 54 shows oropharyngeal swab virus titers of golden Syrian hamsters after treatment with Nanoparticle A prior to cohabitation with SARS-CoV-2-infected animals as described in Example 5.

FIG. 55 shows lung virus titers of golden Syrian hamsters treated with Nanoparticle A and infected with SARS-CoV-2 as described in Example 5.

FIG. 56 shows oropharyngeal swab virus titers of golden Syrian hamsters treated with Nanoparticle A and infected with SARS-CoV-2 as described in Example 5.

FIG. 57 shows for the Transmission Study the percent initial body weight of 5-week-old golden Syrian hamsters following challenge with SARS-CoV-2 and treatment with Nanoparticle A prior to cohabitation with infected animals. (n=4 hamsters/infected group, n=8 hamsters/naïve group) Animals with the same shape symbols were cohabitated. Groups represented with the closed circle and closed square were infected on study day 0. Animals represented by the open circle were naïve and placebo-treated prior to cohabitation with animals from group 1 for 4 hrs per day for 3 days. Animals represented by the open square were naïve and Nanoparticle A-treated prior to cohabitation with animals from group 3. The differences in percent initial body weight were not statistically significant when compared by one-way ANOVA.

FIG. 58 shows for the Transmission Study outlined in Example 5, the lung virus titers of 5-week-old golden Syrian hamsters after challenge with SARS-CoV-2 and treatment with Nanoparticle A prior to cohabitation with infected animals. Animals with the same shape symbols were cohabitated. Groups represented with the closed circle and closed square were infected on study day 0. Animals represented by the open circle were naïve and placebo-treated prior to cohabitation with animals from group 1 for 4 hrs per day for 3 days. Animals represented by the open square were naïve and Nanoparticle A-treated prior to cohabitation with animals from group 3. Treatment with Nanoparticle A significantly reduced lung virus titers in naïve animals cohabitated with SARS-CoV-2-infected animals.

FIG. 59 shows for the Transmission Study outlined in Example 5, the lung weights of 5-week-old golden Syrian hamsters after challenge with SARS-CoV-2 and treatment with Nanoparticle A prior to cohabitation with infected animals. Animals with the same shape symbols were cohabitated. Groups represented with the closed circle and closed square were infected on study day 0. Animals represented by the open circle were naïve and placebo-treated prior to cohabitation with animals from group 1 for 4 hrs per day for 3 days. Animals represented by the open square were naïve and Nanoparticle A-treated prior to cohabitation with animals from group 3. Lung weights were not statistically different between groups when compared by one-way ANOVA.

FIG. 60 shows for the Transmission Study in Example 5, oropharyngeal swab virus titers of 5- week-old golden Syrian hamsters after challenge with SARS-CoV-2 and treatment with Nanoparticle A prior to cohabitation with infected animals. Animals with the same shape symbols were cohabitated. Groups represented with the closed circle and closed square were infected on study day 0. Animals represented by the open circle were naïve and placebo-treated prior to cohabitation with animals from group 1 for 4 hrs per day for 3 days. Animals represented by the open square were naïve and Nanoparticle A-treated prior to cohabitation with animals from group 3. No significant difference in oropharyngeal swab virus titers were determined by one-way ANOVA.

FIG. 61 shows for the Efficacy Study in Example 5, the percent initial body weight of 5-week-old golden Syrian hamsters following treatment with Nanoparticle A and infection with SARS-CoV-2. (n=8 hamsters/group) Treatment with Nanoparticle A started 2 hours prior to infection. The differences in percent initial body weight were not statistically significant when compared by one-way ANOVA.

FIG. 62 shows the lung virus titers of 5-week-old golden Syrian hamsters after treatment with Nanoparticle A and infection with SARS-CoV-2. Treatment with Nanoparticle A started significantly reduced lung virus titers at doses of 2 and 0.63 mg/kg/d compared to placebo-treated animals. (**P<0.01 compared to placebo-treated animals.)

FIG. 63 shows the lung weights of 5-week-old golden Syrian hamsters after challenge with SARS-CoV-2 and treatment with Nanoparticle A prior to cohabitation with infected animals. Lung weights were not statistically different between groups when compared by one-way ANOVA.

FIG. 64 shows for the Efficacy Study in Example 5, oropharyngeal swab virus titers of 5-week-old golden Syrian hamsters after treatment with Nanoparticle A and infection with SARS-CoV-2. Treatment with Nanoparticle A at a dose of 2 mg/kg/d significantly reduced oropharyngeal swab titers of hamsters infected with SARS-CoV-2. (*P<0.05 compared to placebo-treated animals.)

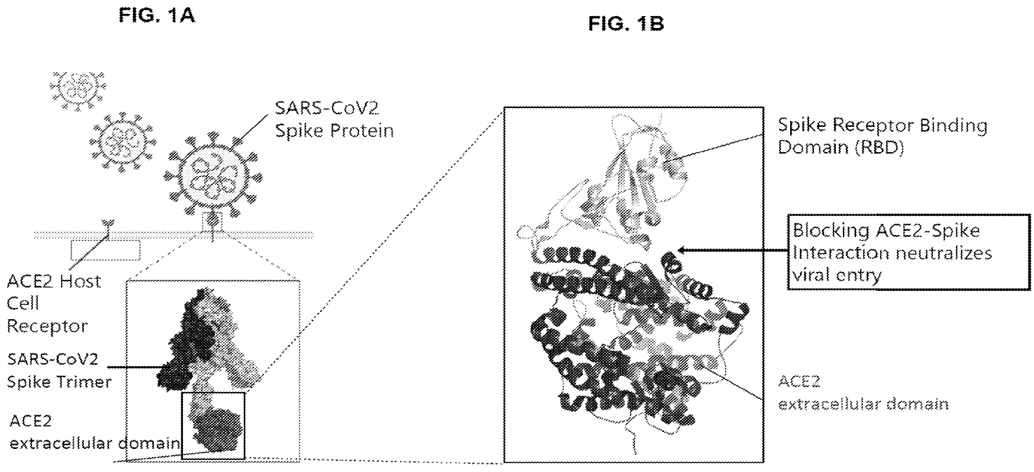

### III. DETAILED DESCRIPTION OF THE INVENTION

A. Definitions

By “amino acid” and “amino acid identity” as used herein is meant one of the 20 naturally occurring amino acids or any non-natural analogues that may be present at a specific, defined position. In many embodiments, “amino acid” means one of the 20 naturally occurring amino acids. By “protein” herein is meant at least two covalently attached amino acids, which includes proteins, polypeptides, oligopeptides and peptides.

By “amino acid modification” herein is meant an amino acid substitution, insertion, and/or deletion in a polypeptide sequence or an alteration to a moiety chemically linked to a protein. For example, a modification may be an altered carbohydrate or PEG structure attached to a protein. For clarity, unless otherwise noted, the amino acid modification is always to an amino acid coded for by DNA, e.g. the 20 amino acids that have codons in DNA and RNA. The preferred amino acid modification herein is a substitution.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence.

By “amino acid deletion” or “deletion” as used herein is meant the removal of an amino acid sequence at a particular position in a parent polypeptide sequence.

The polypeptides of the invention specifically bind to the Spike trimeric protein as outlined herein. “Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antigen binding domain (SBD) having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, at least about 10⁻¹³ M, at least about 10⁻¹⁴ M, at least about 10⁻¹⁵ M or greater, where KD refers to a dissociation rate of a particular ABD-antigen interaction. Typically, an ABD that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore assay or Octet as is known in the art.

By “parent polypeptide” or “precursor polypeptide” (including the renumerated anti-Spike antigen binding domains of the invention) as used herein is meant a polypeptide that is subsequently modified to generate a variant. In this case, for example, any one of the starting clones of FIG. 13 can be considered a “parent polypeptide” as is the case of AeroNab6. Parent polypeptide may refer to the polypeptide itself, compositions that comprise the parent polypeptide, or the amino acid sequence that encodes it.

The polypeptides of the invention have at least about 90%, 91, 92, 92, 94, 95, 96, 97, 98, 99, 99.2. 99.4. 99.6. 99.8 or 100% sequence identity with a sequence set forth herein.

By “position” as used herein is meant a location in the sequence of a protein. Positions may be numbered sequentially, or according to an established format.

By “variable heavy domain” or “VH domain” or “VHH domain” herein is meant the region of the antigen binding domain that contains the CDRs. The molecules discussed herein do not contain VL domains. In these embodiments, each VH is composed of three hypervariable regions (“complementary determining regions,” “CDRs”) and four “framework regions”, or “FRs”, arranged from amino-terminus to carboxy-terminus in the following order: FR1-vhhCDR1-FR2-vhhCDR2-FR3-vhhCDR3-FR4. The vhFR regions self-assemble to form the sdABD that are Fv domains. By “single domain Fv”, “sdFv” or “sdABD” herein is meant an antigen binding domain that only has three CDRs, generally based on camelid antibody technology. See: Protein Engineering 9(7):1129-35 (1994); Rev Mol Biotech 74:277-302 (2001); Ann Rev Biochem 82:775-97 (2013). sdABDs are distinguished from single domain antibodies by the lack of the constant domains (in the case of camelid antibodies, the CH2-CH3 domains).

The hypervariable regions confer antigen binding specificity and generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et at, SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

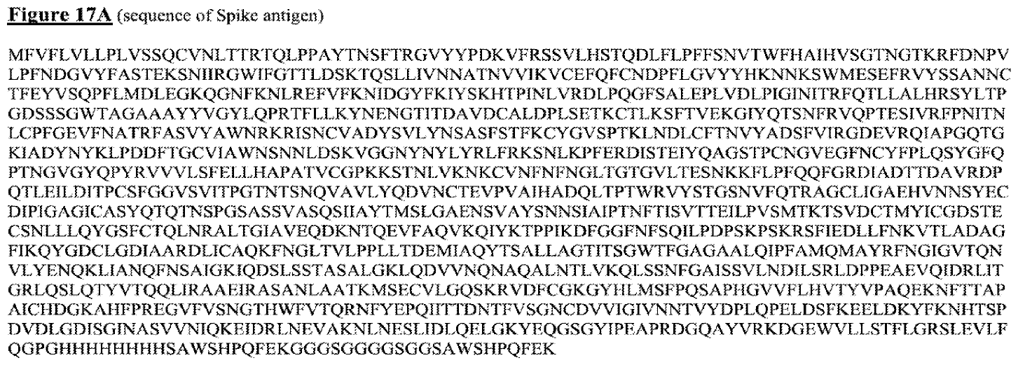

As described herein the spike antigen is defined by the sequence found in FIG. 17A and/or the following sequence: MFVFLVLLPLVSSQCVNLTTRTQLPPAYTNSFTRGVYYPDKVFRSSVLHSTQDLFLPFFSNVTWFH AIHVSGTNGTKRFDNPVLPFNDGVYFASTEKSNIIRGWIFGTTLDSKTQSLLIVNNATNVVIKVCE FQFCNDPFLGVYYHKNNKSWMESEFRVYSSANNCTFEYVSQPFLMDLEGKQGNFKNLREFVFK NIDGYFKIYSKHTPINLVRDLPQGFSALEPLVDLPIGINITRFQTLLALHRSYLTPGDSSSGWTAGA AAYYVGYLQPRTFLLKYNENGTITDAVDCALDPLSETKCTLKSFTVEKGIYQTSNFRVQPTESIVR FPNITNLCPFGEVFNATRFASVYAWNRKRISNCVADYSVLYNSASFSTFKCYGVSPTKLNDLCFT NVYADSFVIRGDEVRQIAPGQTGKIADYNYKLPDDFTGCVIAWNSNNLDSKVGGNYNYLYRLF RKSNLKPFERDISTEIYQAGSTPCNGVEGFNCYFPLQSYGFQPTNGVGYQPYRVVVLSFELLHAPA TVCGPKKSTNLVKNKCVNFNFNGLTGTGVLTESNKKFLPFQQFGRDIADTTDAVRDPQTLEILDI TPCSFGGVSVITPGTNTSNQVAVLYQDVNCTEVPVAIHADQLTPTWRVYSTGSNVFQTRAGCLI GAEHVNNSYECDIPIGAGICASYQTQTNSPRRARSVASQSIIAYTMSLGAENSVAYSNNSIAIPTN FTISVTTEILPVSMTKTSVDCTMYICGDSTECSNLLLQYGSFCTQLNRALTGIAVEQDKNTQEVFA QVKQIYKTPPIKDFGGFNFSQILPDPSKPSKRSFIEDLLFNKVTLADAGFIKQYGDCLGDIAARDLI CAQKFNGLTVLPPLLTDEMIAQYTSALLAGTITSGWTFGAGAALQIPFAMQMAYRFNGIGVTQN VLYENQKLIANQFNSAIGKIQDSLSSTASALGKLQDVVNQNAQALNTLVKQLSSNFGAISSVLN DILSRLDKVEAEVQIDRLITGRLQSLQTYVTQQLIRAAEIRASANLAATKMSECVLGQSKRVDFCG KGYHLMSFPQSAPHGVVFLHVTYVPAQEKNFTTAPAICHDGKAHFPREGVFVSNGTHWFVTQR NFYEPQIITTDNTFVSGNCDVVIGIVNNTVYDPLQPELDSFKEELDKYFKNHTSPDVDLGDISGIN ASVVNIQKEIDRLNEVAKNLNESLIDLQELGKYEQYIKWPWYIWLGFIAGLIAIVMVTIMLCCMT SCCSCLKGCCSCGSCCKFDEDDSEPVLKGVKLHYT (SEQ ID NO: 300). This sequence is interpreted to include variants arising from mutations in SARS-CoV-2 that might arise from time to time. In various embodiments, the variants differ from the above sequence in the ACE2 binding domain of the spike protein. In some embodiments, the variants differ from the above sequence at a site other than the ACE2 binding domain. In some embodiments, the variants differ from the above sequence in at least the ACE2 binding domain and one other site. In various embodiments, the variant is different than the above sequence by at least 1, 2, 3, 4, 5, 6, 7, 8, 9, 10 or more amino acids.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3).

A useful comparison of CDR numbering is as below, see Lafranc et al., Dev. Comp. Immunol. 27(1):55-77 (2003):

TABLE 1

| | Kabat + | | | | | |
|---|---|---|---|---|---|---|
| | Chothia | IMGT | Kabat | AbM | Chothia | Contact |
| vhCDR1 | 26-35 | 27-38 | 31-35 | 26-35 | 26-32 | 30-35 |
| vhCDR2 | 50-65 | 56-65 | 50-65 | 50-58 | 52-56 | 47-58 |
| vhCDR3 | 95-102 | 105-117 | 95-102 | 95-102 | 95-102 | 93-101 |
| vlCDR1 | 24-34 | 27-38 | 24-34 | 24-34 | 24-34 | 30-36 |
| vlCDR2 | 50-56 | 56-65 | 50-56 | 50-56 | 50-56 | 46-55 |
| vlCDR3 | 89-97 | 105-117 | 89-97 | 89-97 | 89-97 | 89-96 |

Throughout the present specification, the IMGT numbering system is generally used when referring to a residue in the variable domain.

The present invention provides a large number of different CDR sets which can be assembled into sdABDs. In the context of a single domain ABD (“sdABD”), a CDR set is only three CDRs; these are sometimes referred to in the art as “VHH” domains as well.

The CDRs contribute to the formation of the antigen-binding, or more specifically, epitope binding sites. “Epitope” refers to a determinant that interacts with a specific antigen binding site in the variable regions known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specific antigen binding peptide; in other words, the amino acid residue is within the footprint of the specific antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

B. Introduction

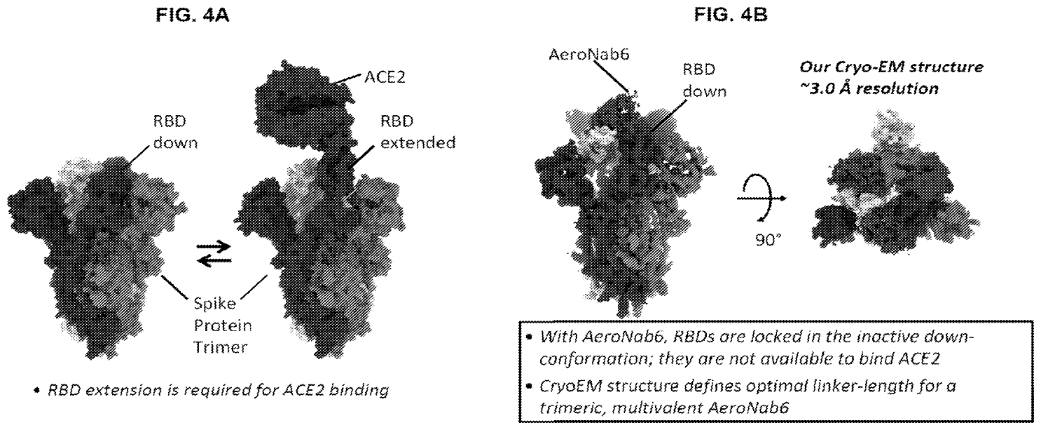

As is known in the art, coronaviruses are enveloped positive-stranded RNA viruses that relies on membrane fusion as an early step for entering host cells. Additionally, the surfaces of many coronaviruses, and SARS-CoV2 in particular, are decorated with a Spike glycoprotein. The Spike protein forms a homotrimeric complex of three identical Spike protein monomers that can be functionally categorized as having two distinct subunits, S1 and S2. The S1 subunit contains the receptor binding domain (RBD) which binds to the ACE2 receptor on human cells, while the S2 subunit is involved in the fusion of the viral and cellular membranes. The RBDs of the trimeric complex can be in two different conformations: an extended, or “up” conformation (sometimes also referred to herein as the “on” conformation), as depicted in FIG. 4, that is accessible for binding to the ACE2 complex, and a “down” or “off” conformation, again depicted in FIG. 4, which represents a receptor-inaccessible state. That is, the Spike protein cannot bind to the ACE2 receptor and infect cells when in the “down” conformation.

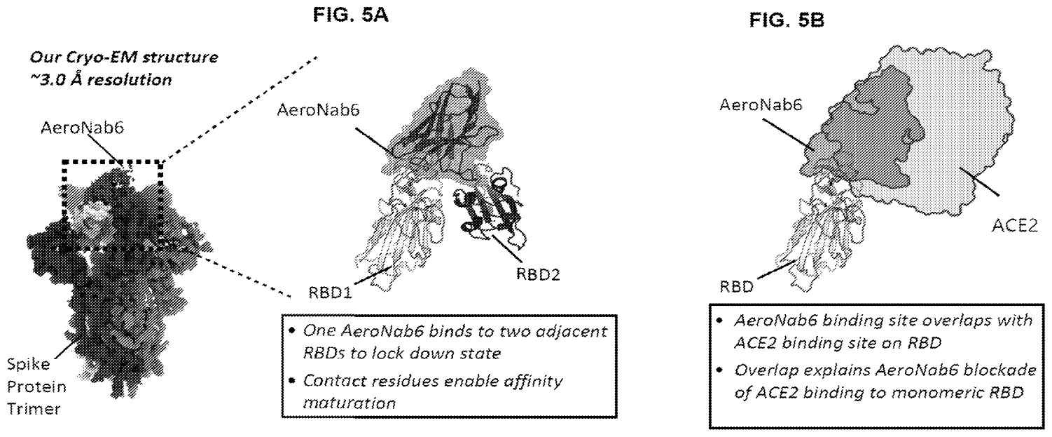

The present invention is directed to antigen binding domains that not only bind to the Spike protein, but bind in such a way as to “lock” the Spike protein in the “off” or “down” position with extremely high affinity. As is shown in FIG. 5, the binding of the MASC proteins of the invention actually bind to two different RBDs (of the three present in the trimer), thus not only keeping the Spike protein “off” but also occupying a part of the ACE2 binding site to further prevent membrane fusion and infection.

The present invention provides Multivalent Anti-SARS-CoV2 (“MASC”) fusion proteins to antigen binding domains (ABDs) that bind in a multivalent way to the trimeric Spike protein of the SARS-CoV2 virus with very high affinity. The antigen binding domains are based on single domain antibodies (sdAbs) that contain a single variable heavy domain (frequently referred to in the field as “VHH” domains) instead of the typical variable heavy and variable light domains of traditional antibodies. The single domain antigen binding domains (sdABDs) confer a number of advantages in their use to bind to viral proteins, as they are significantly smaller than traditional ABDs, have generally high thermal stability, and increased solubility, as further discussed below.

Additionally, as has been shown for other sdABDs, the sdABDs of the present invention can be assembled into multimeric structures such as dimers and trimers, similar to other “Nanobodies™”; see generally U.S. Pat. No. 9,834,595. Thus, the present invention provides multivalent anti-SARS-CoV2 (“MASC”) fusion proteins that contain sdABDs linked together through domain linkers as is more fully described below that bind the Spike protein and prevent viral entry into human cells via the ACE2 receptor, as is further discussed below.

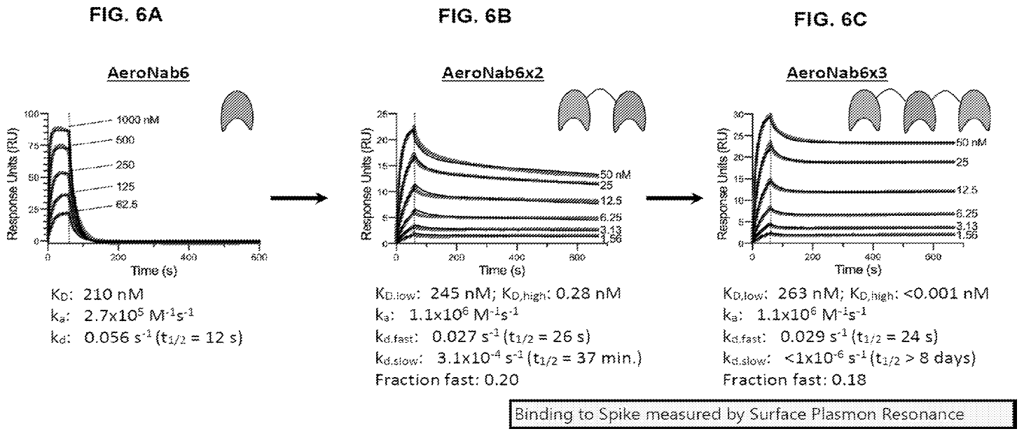

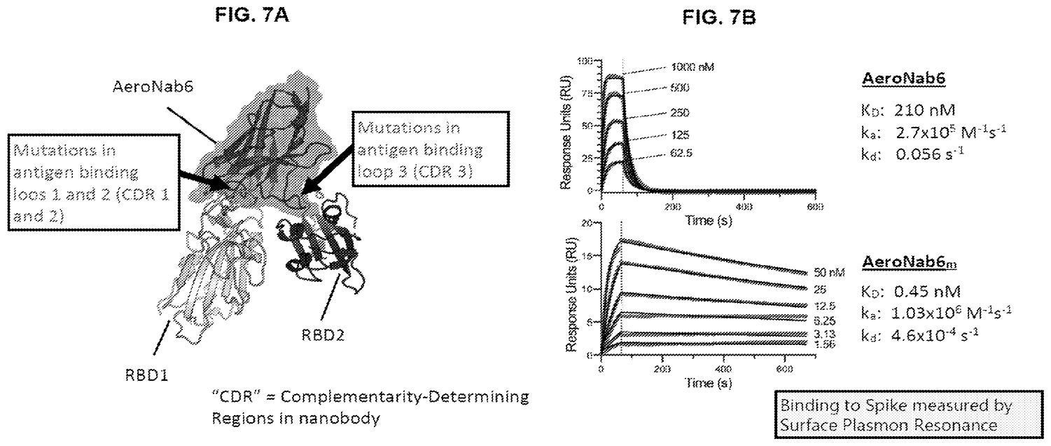

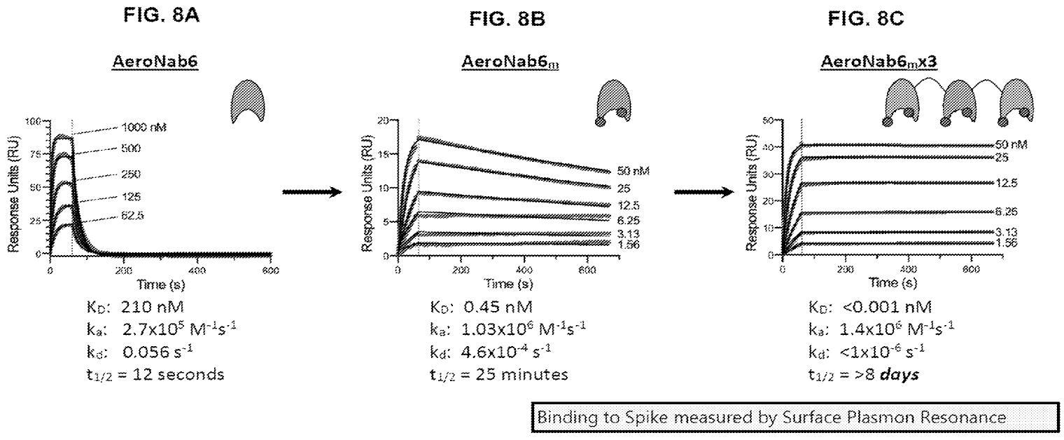

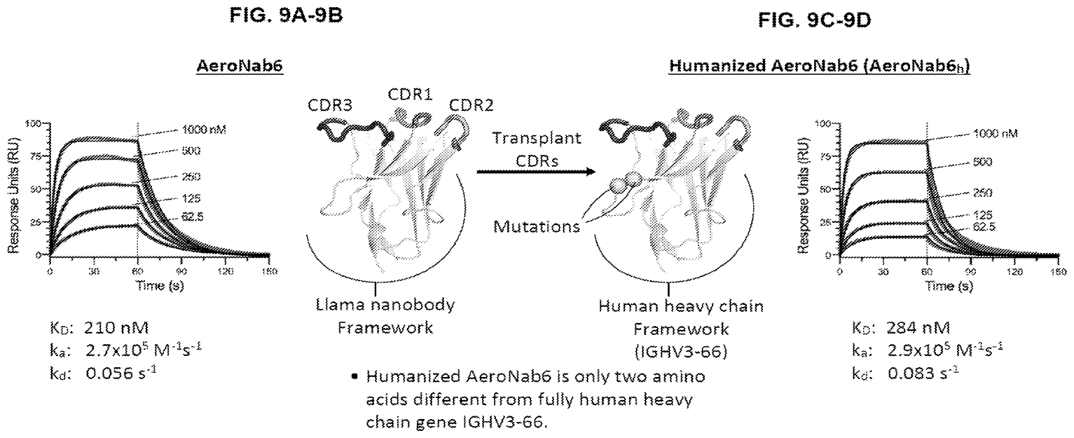

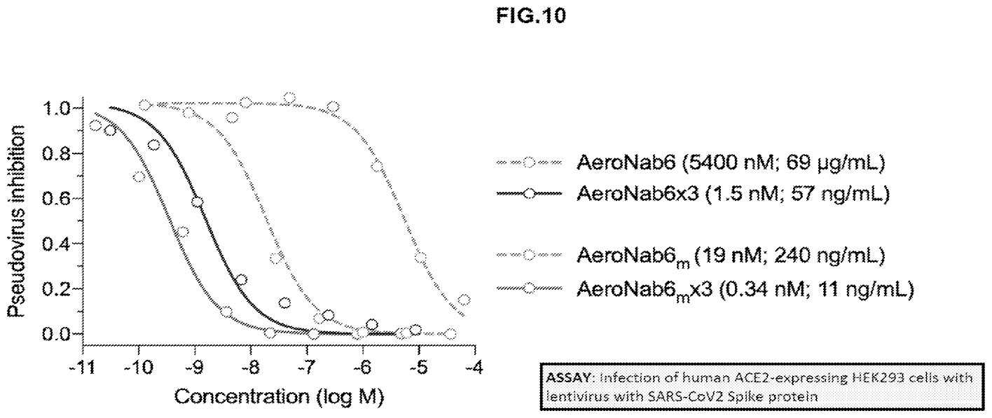

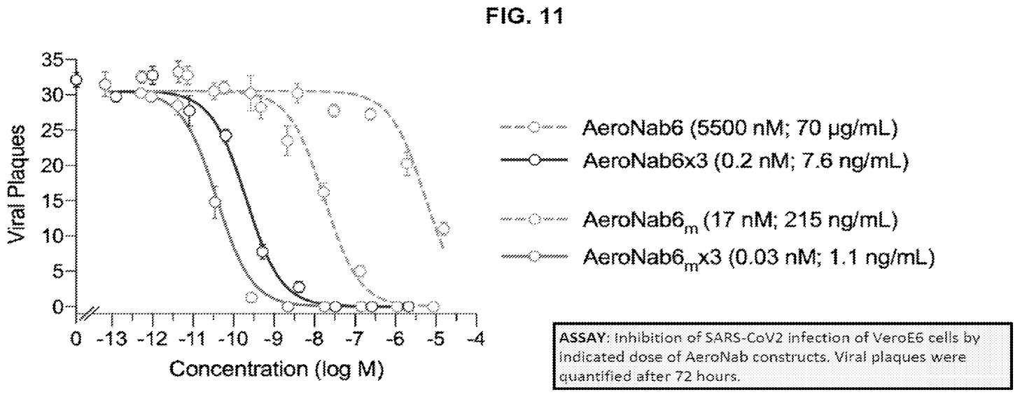

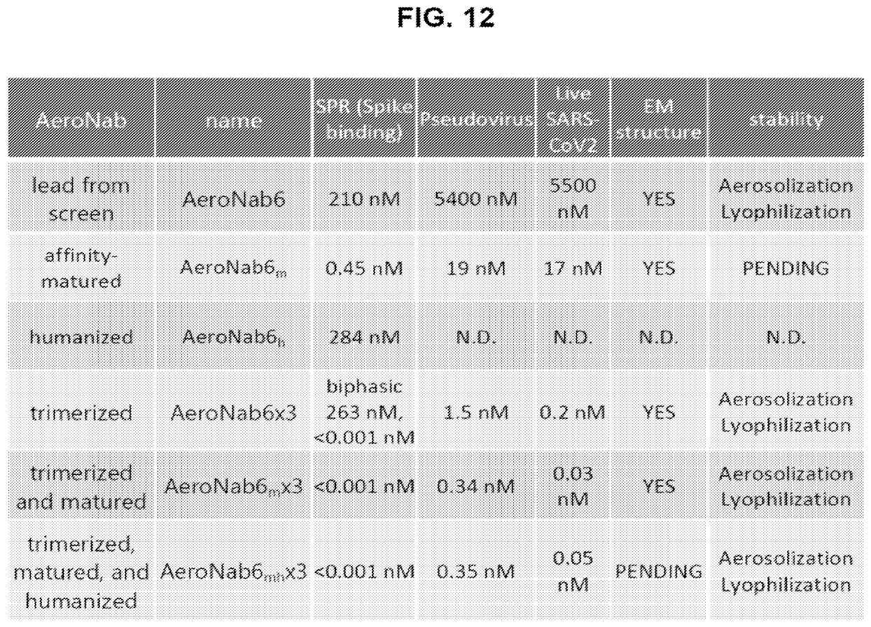

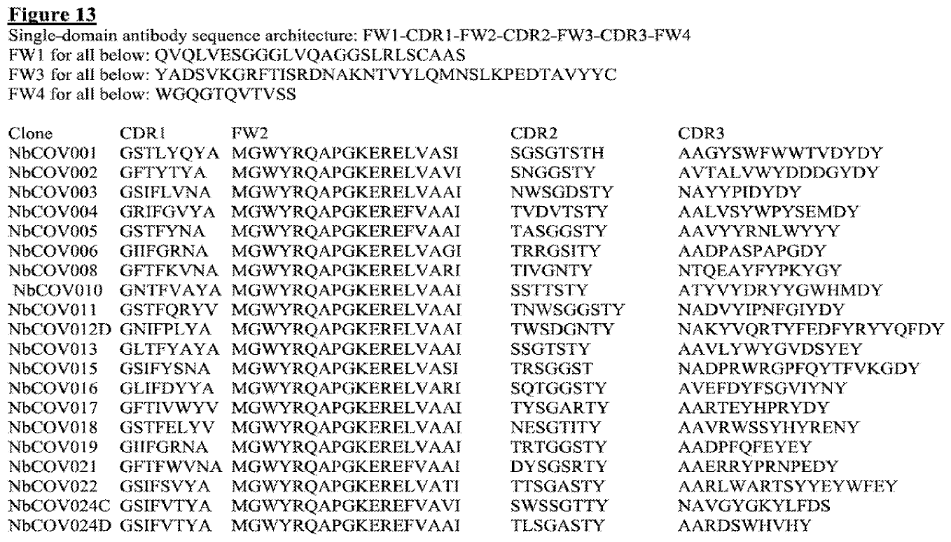

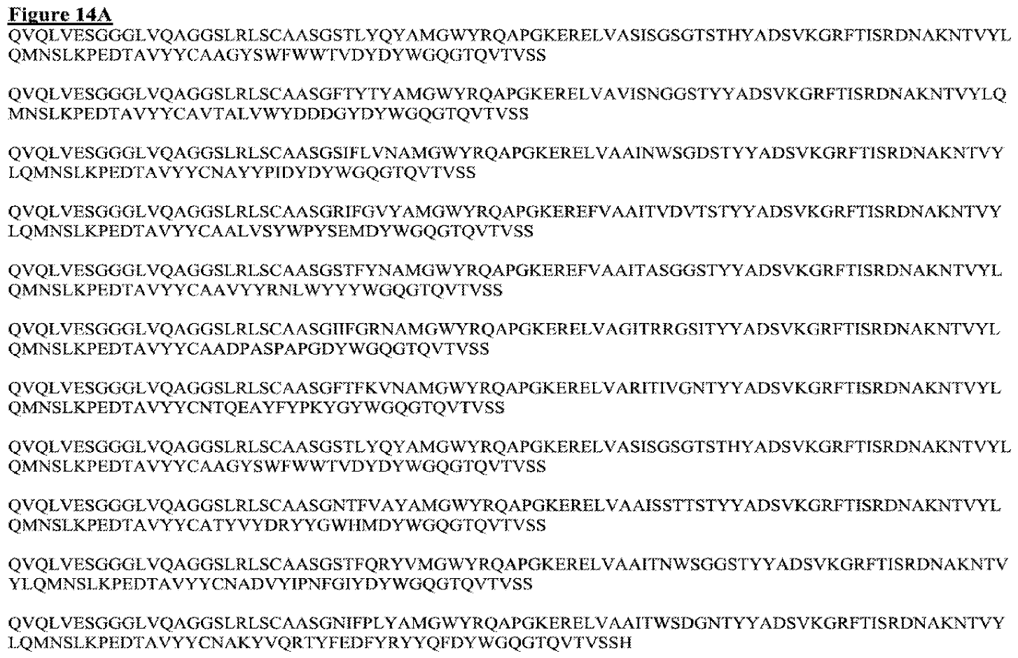

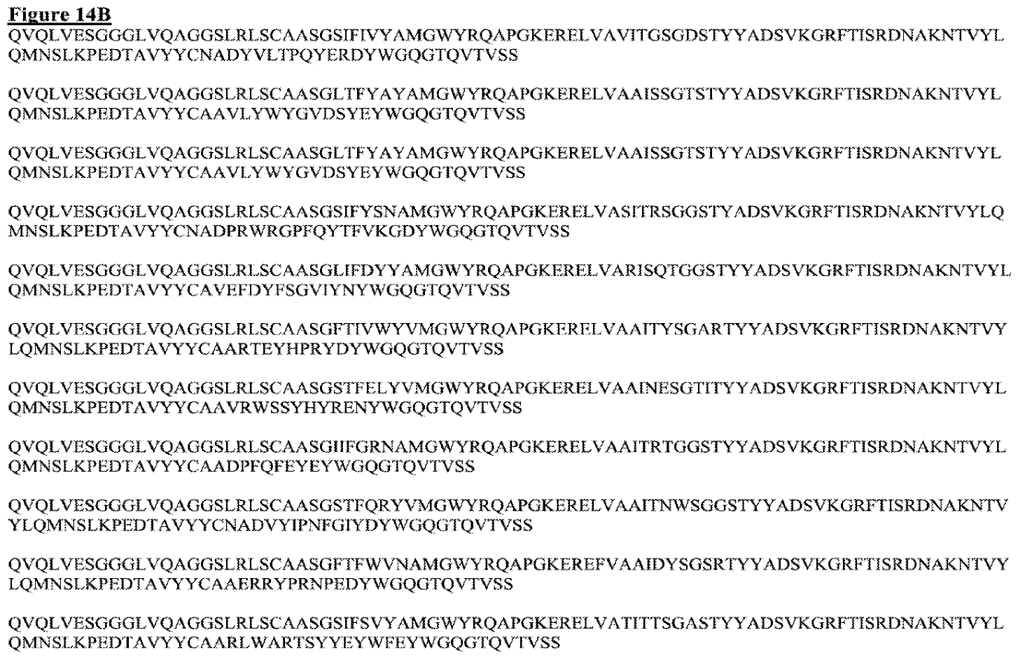

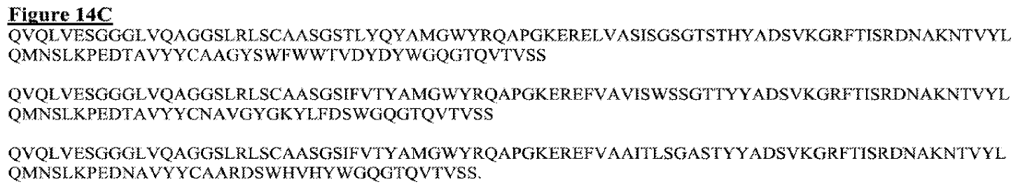

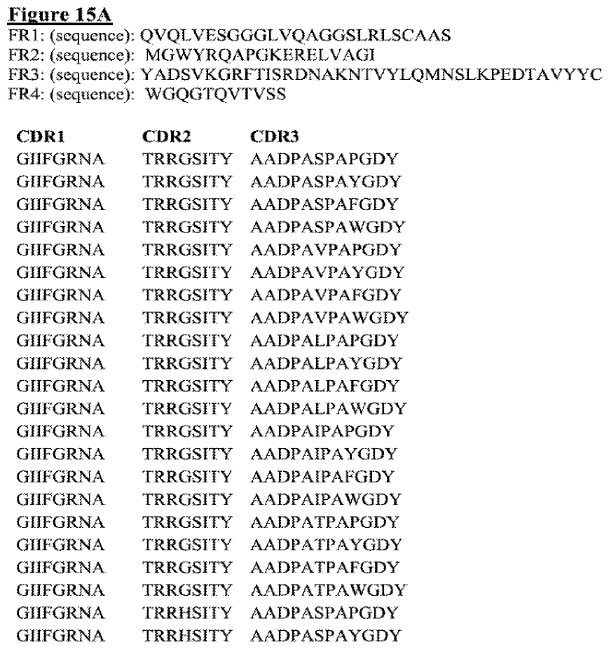

In these multivalent embodiments, the fusion proteins of the invention have a several different components, generally referred to herein as domains, which are linked together in a variety of ways depending on the format. Some of the domains are binding domains, that each bind to the target Spike protein, and some are domain linkers. Thus, as generally pictured in FIG. 6, the present invention provides for MASC proteins that comprise a single sdABD that binds the RBD, as well as MASC fusion proteins that contain two sdABDs linked using a domain linker (sdABD-domain linker-sdABD) and MASC fusion proteins that contain three sdABDs linked with domain linkers (sdABD-domain linker-sdABD-domain linker-sdABD). As discussed below, these domain linkers can be the same or different.

Another distinct advantage of the MASC fusion proteins of the invention is that due to their significant thermal and structure stability, the MASC fusion proteins can be lyophilized and/or aerosolized while retaining binding and neutralization functions. The possibility of administering these MASC fusion proteins directly to the pulmonary system is particularly useful in the case of the SARS-CoV2 virus, as it is known to act specifically in the lungs.

Accordingly, the present invention provides MASC proteins and MASC fusion proteins as is further described herein.

C. Multivalent Anti-SARS-CoV2 (“MASC”) Proteins

Accordingly, the present invention provides MASC proteins that can take several different formats. As discussed herein, the MASC proteins can be a single sdABD as outlined herein, sometimes referred to herein as “monomeric MASC proteins”. MASC proteins can also be linked together to form dimers and trimers as discussed herein. In these embodiments, the dimers and trimers are referred to generally as “MASC fusion proteins”, and there are domain linkers between the monomers.

As will be appreciated by those in the art and more fully described below, in addition to multimers of a single sdABD, multimers can also be made using sdABDs with different binding affinities or properties. That is, a dimeric MASC fusion protein can be “homodimeric”, with the sdABDs having the identical CDRs and/or sequence, or “heterodimeric”, where one sdABD has one set of CDRs and the other has a different set of CDRs. Similarly, trimeric MASC fusion proteins can be homotrimers, or they can be heterotrimers, either utilizing two different sdABDs with different CDRs (two of one and one of the other in the trimer), or heterotrimers with three different CDR sets.

Additionally, as more fully discussed below, the MASC fusion proteins can also include an additional domain that serves to extend the half-life of the MASC protein in plasma. Thus, for example, monomeric, dimeric or trimeric MASC proteins can be fused to a half-life extension domain.

As discussed herein, 21 different clones were originally made, representing a wide variety of antigen binding domains as depicted in FIG. 13. All of these clones bound the spike trimer.

Furthermore, as discussed herein, any of the parental MASC proteins shown in FIG. 13 can undergo affinity maturation. An exemplary example is the affinity maturation of AeroNab6, discussed herein. An affinity maturation campaign resulted in a number of changes in the vhhCDRs, all of which can be combined.

Similarly, as will be appreciated by those in the art, the parental MASC proteins of FIG. 13, or affinity matured MASC proteins can also be humanized. Humanization techniques are well known in the art.

As further discussed below, AeroNab6 MASC makes extensive contacts within the ACE2 binding region of the SC2 spike RBD, including residues 446, 447, 449, 453, 455, 456, 483-486, 489-490, 493-496, 498, 501, and 505). The CDR3 of AeroNab6 MASC contacts a neighboring RBD on the SC2 spike at a three dimensional epitope defined by residues 342, 343, 367, 371-375, 404, 436-441. This additional contact enables AeroNab6 MASC to locking the neighboring RBD in the “off” position, while simultaneously disrupting ACE2 binding at an adjacent RBD.

Additionally, it is known in the art that there can be immunogenicity in humans originating from the C-terminal sequences of certain ABDs. Accordingly, in general, when the C-terminus of the constructs terminates in an sdABD such as depicted here, a histidine tag (either His6 or His10) can be used. These in some cases can be used as purification tags as well, but these sequences can also be used to reduce immunogenicity in humans, as is shown by Holland et al., DOI 10.1007/s10875-013-9915-0 and WO2013/024059.

1. Monomeric Constructs

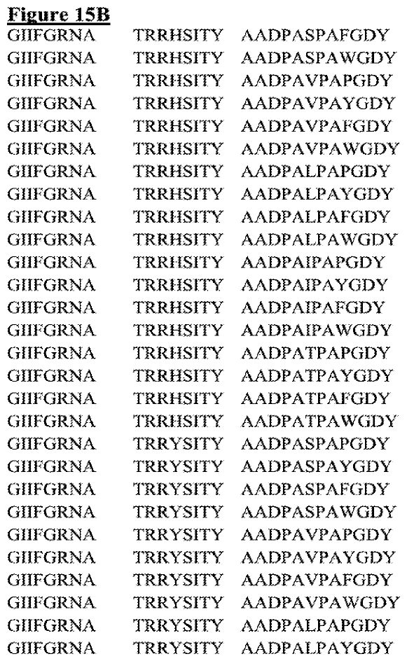

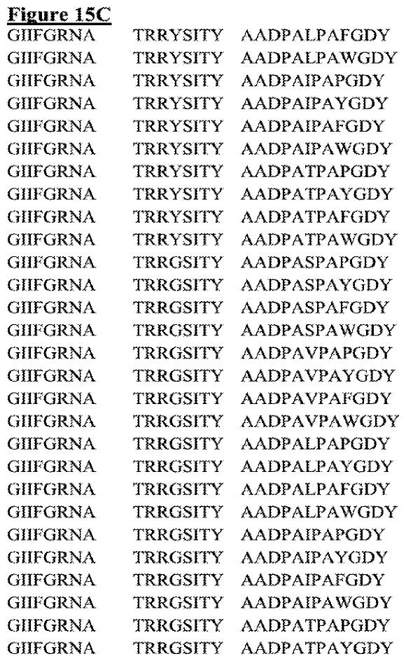

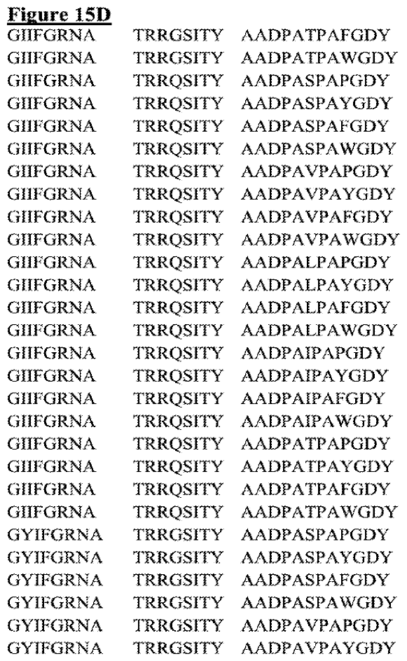

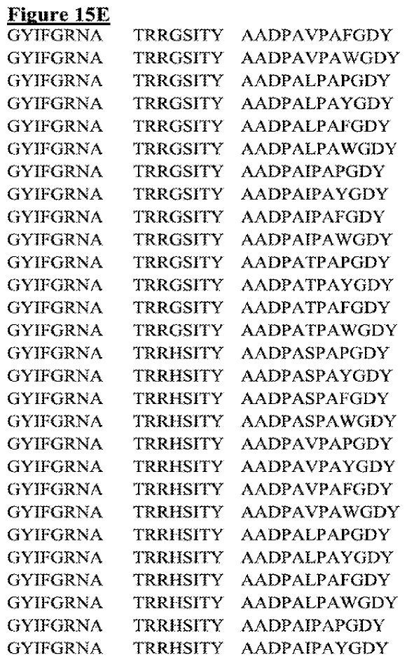

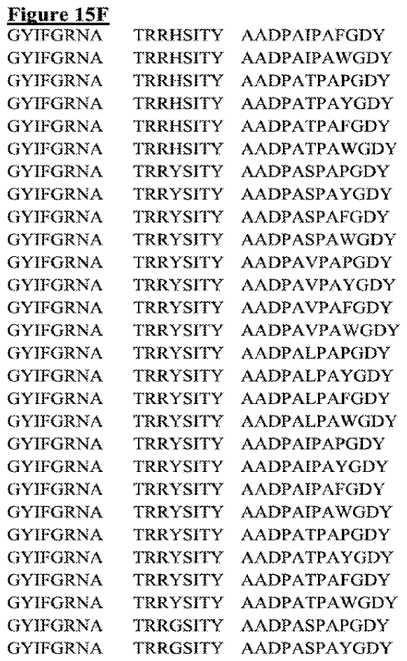

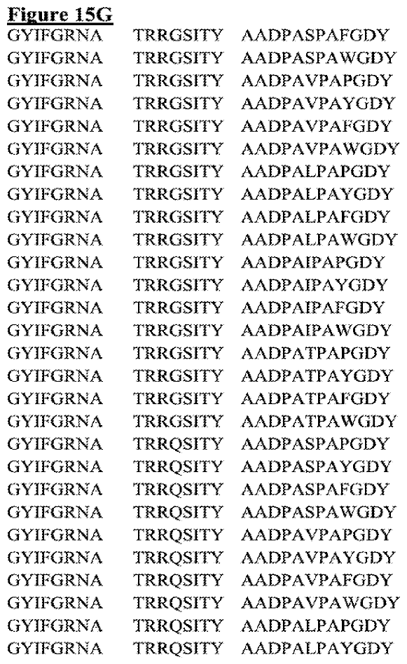

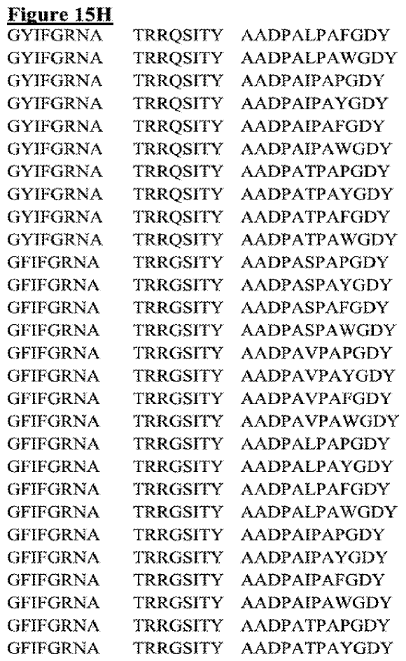

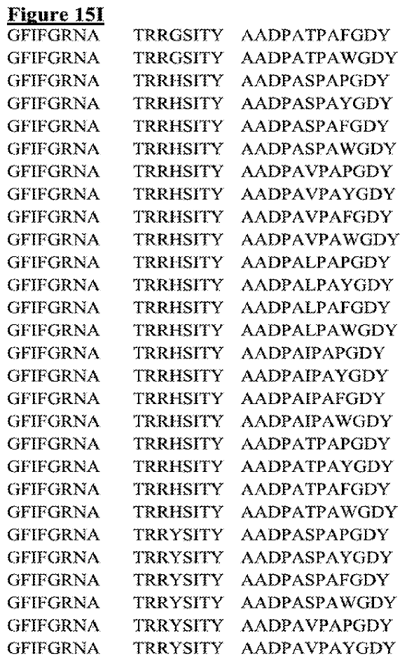

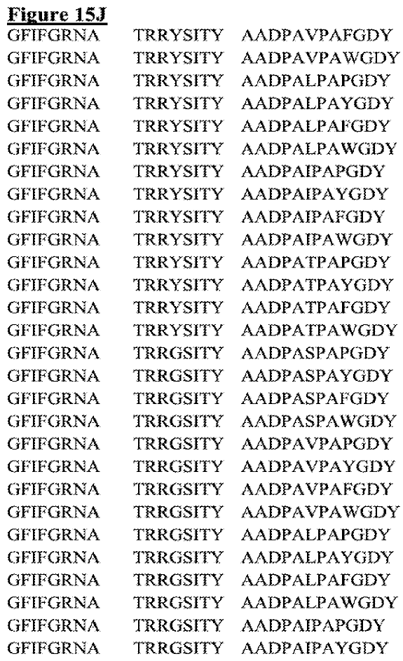

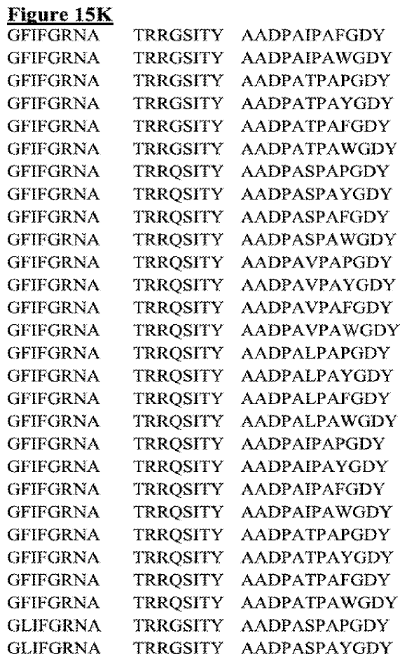

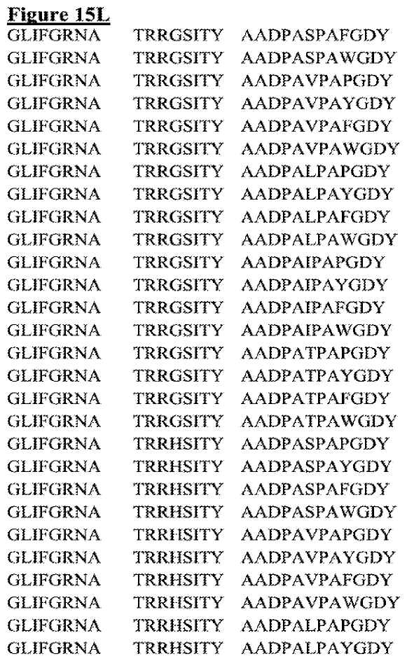

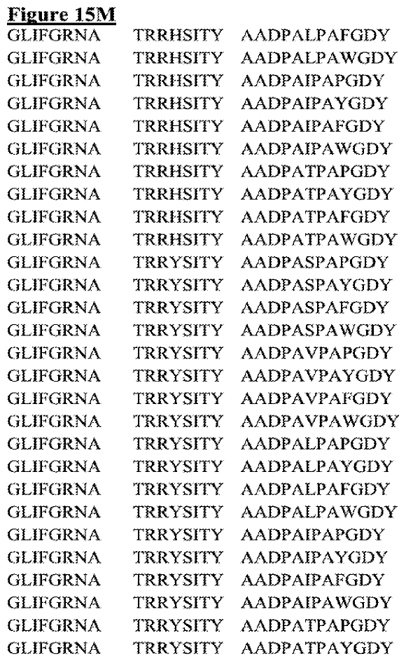

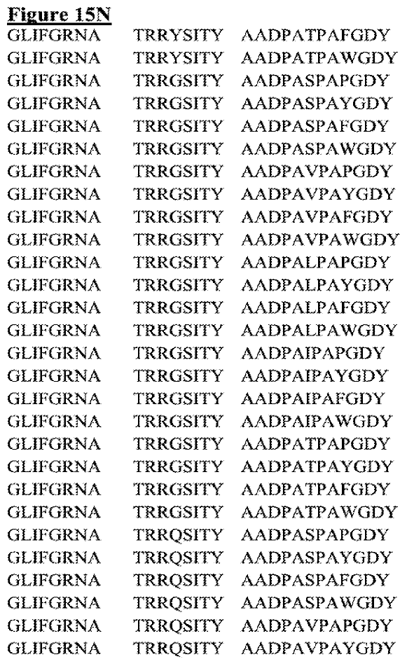

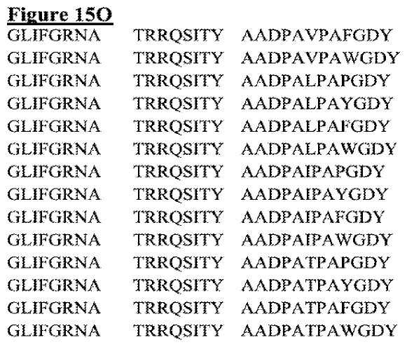

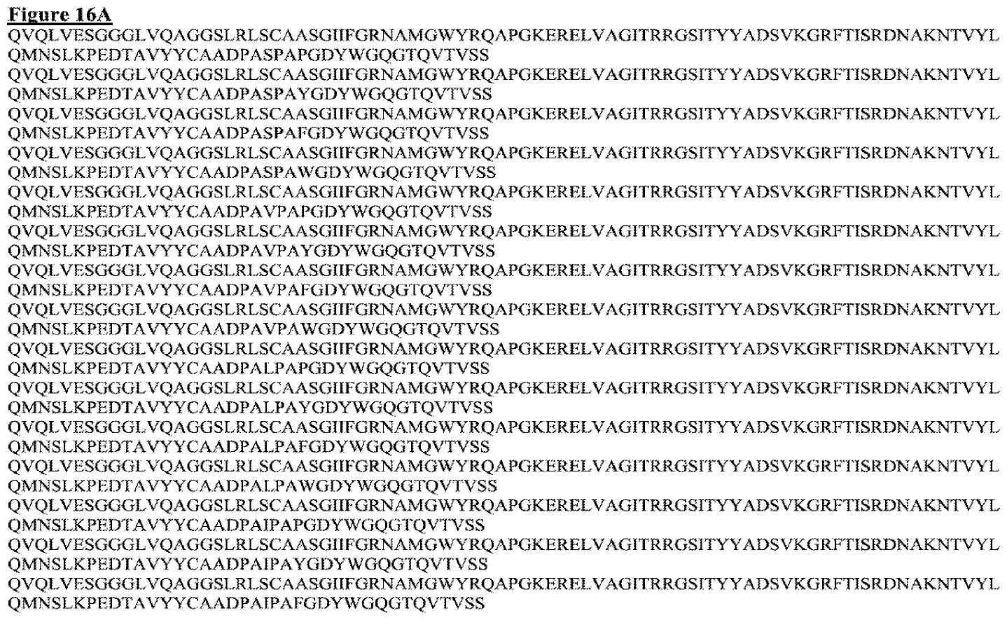

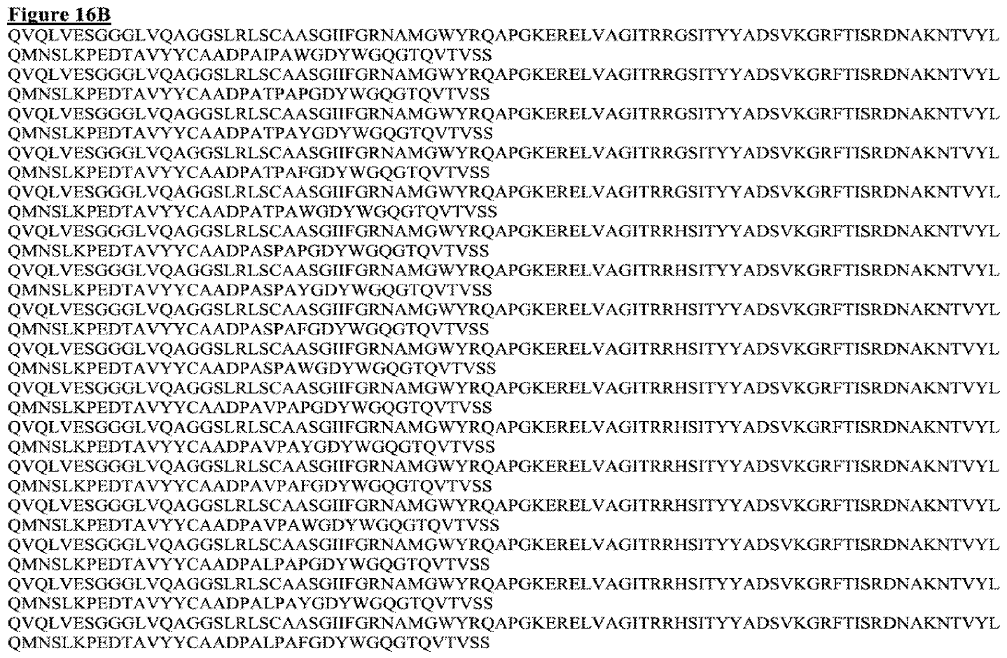

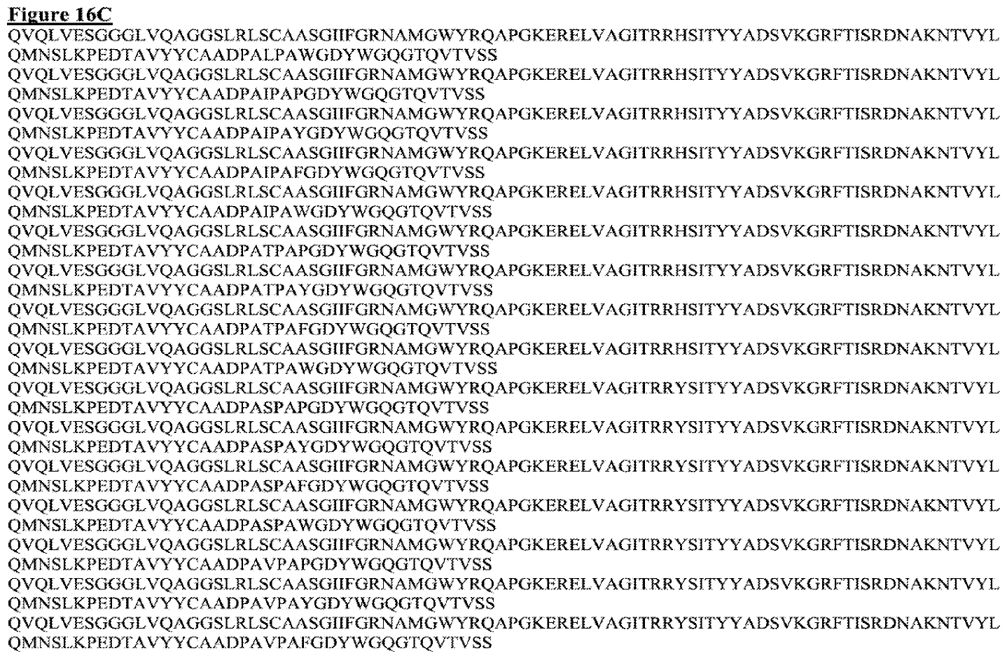

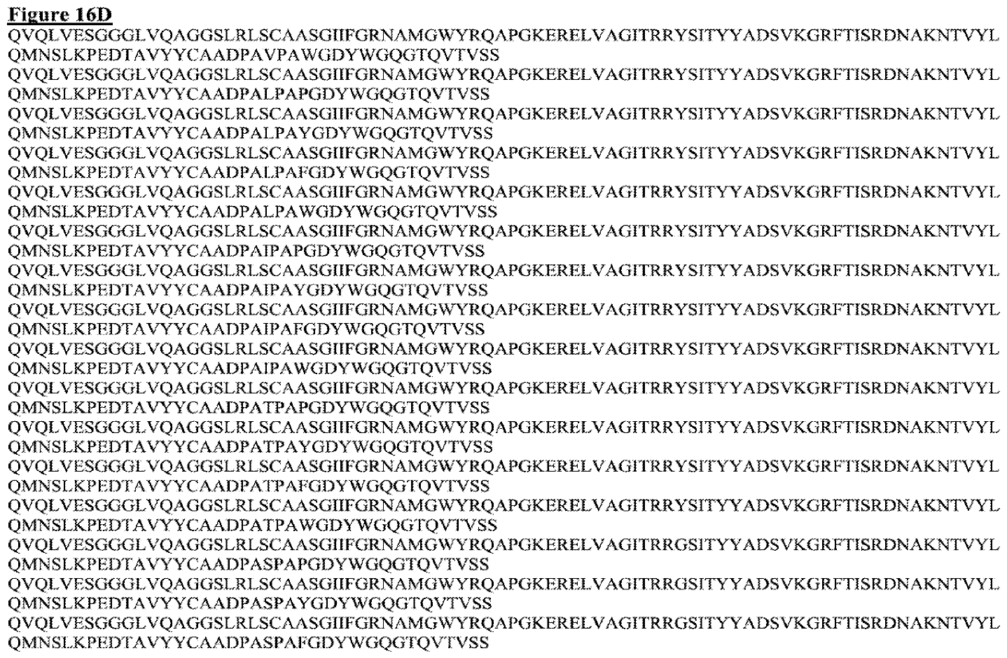

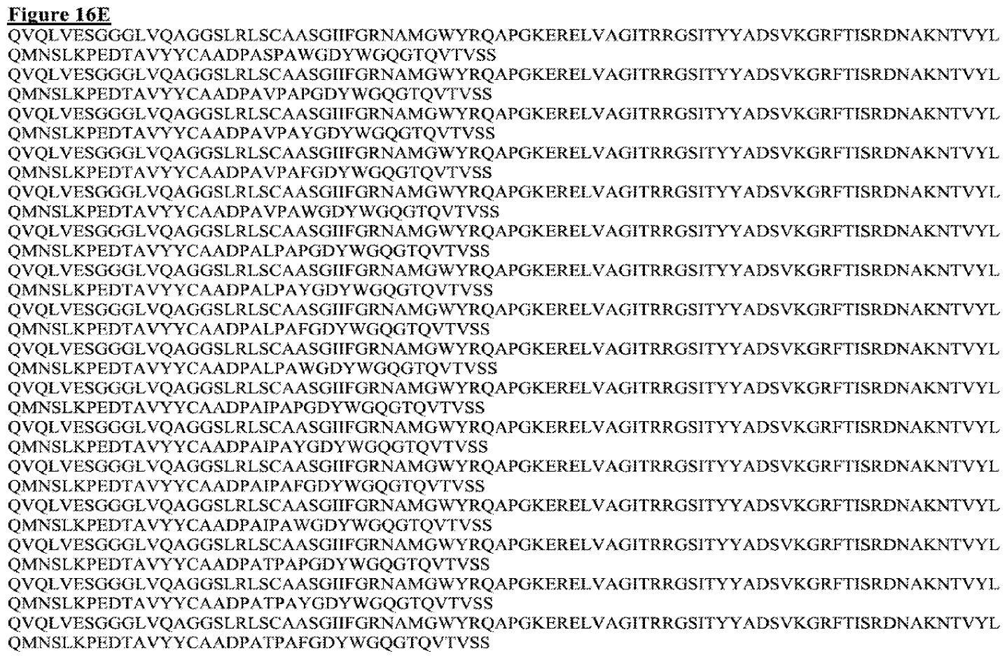

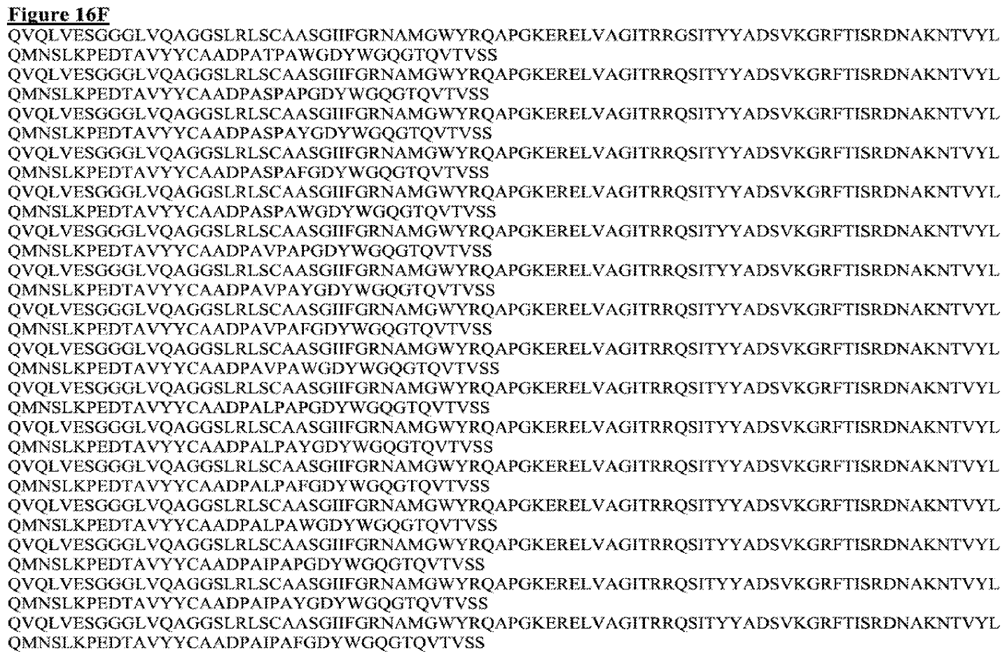

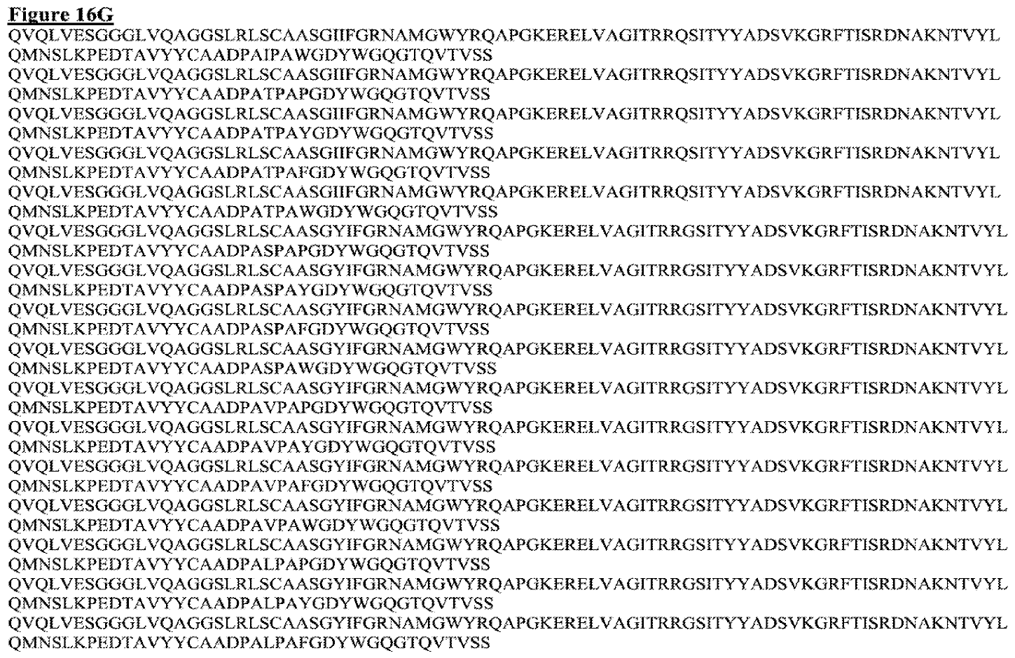

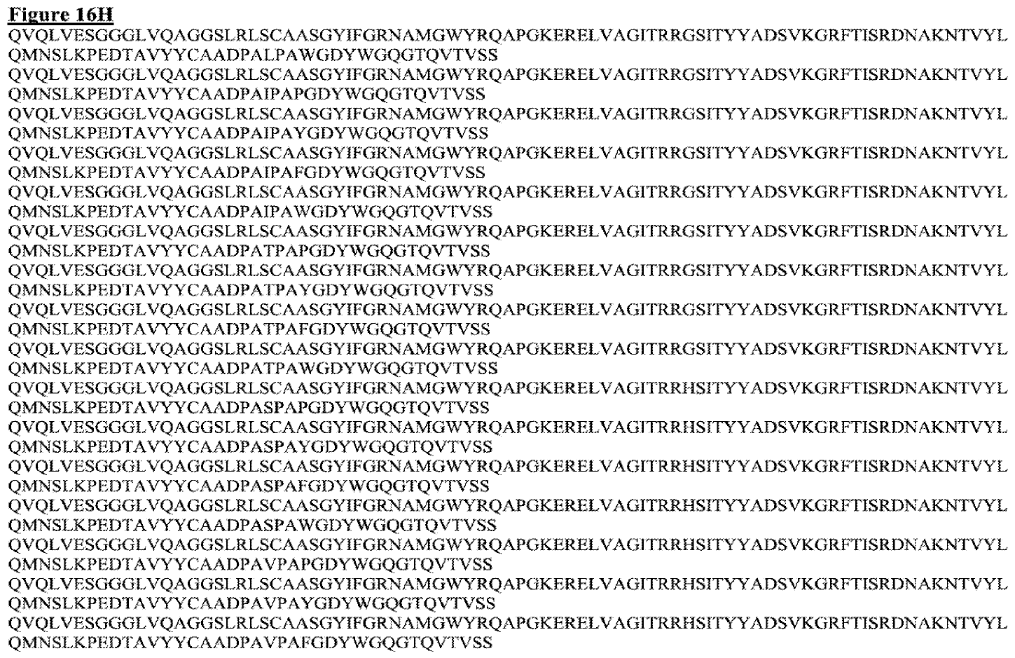

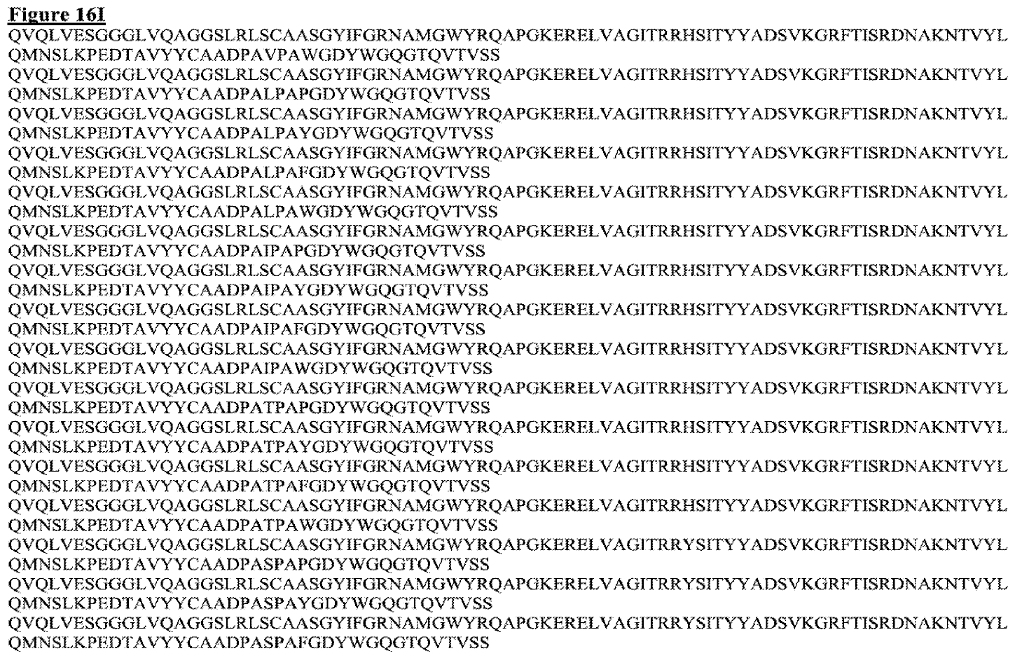

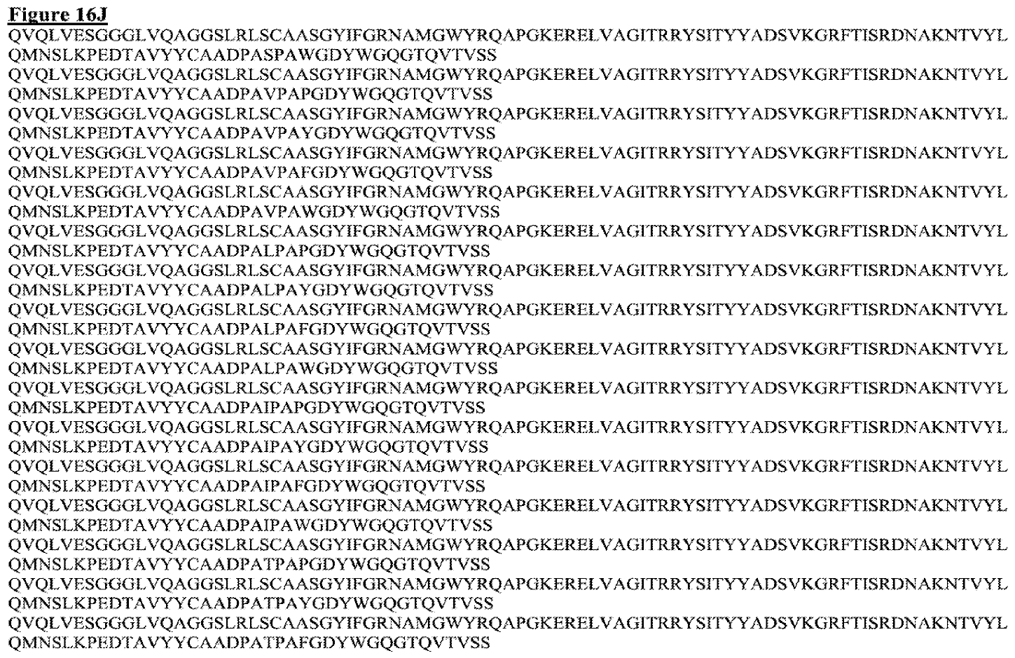

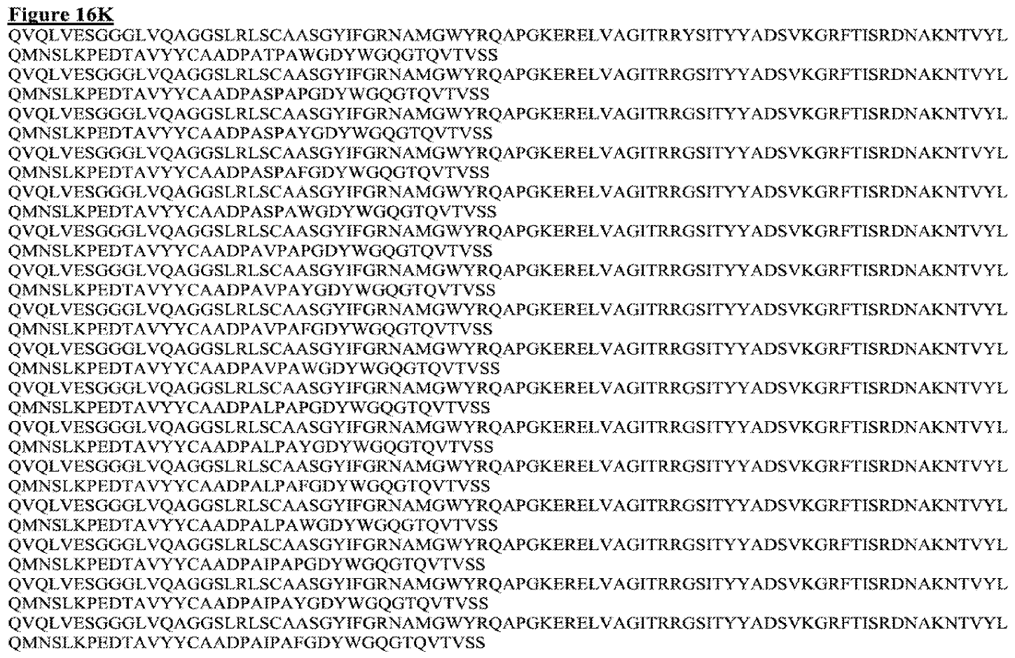

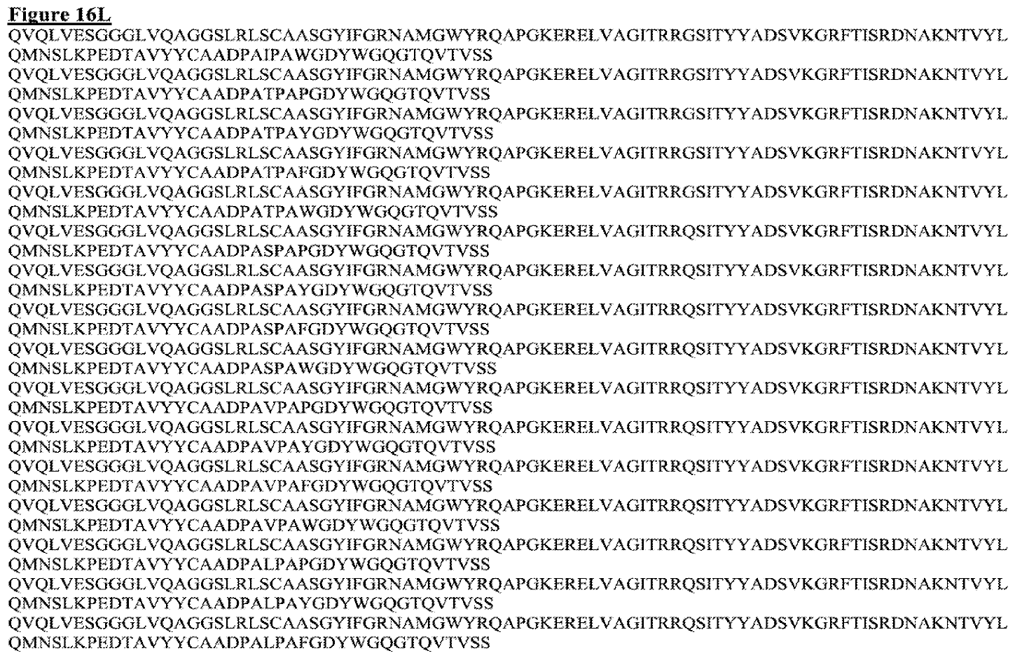

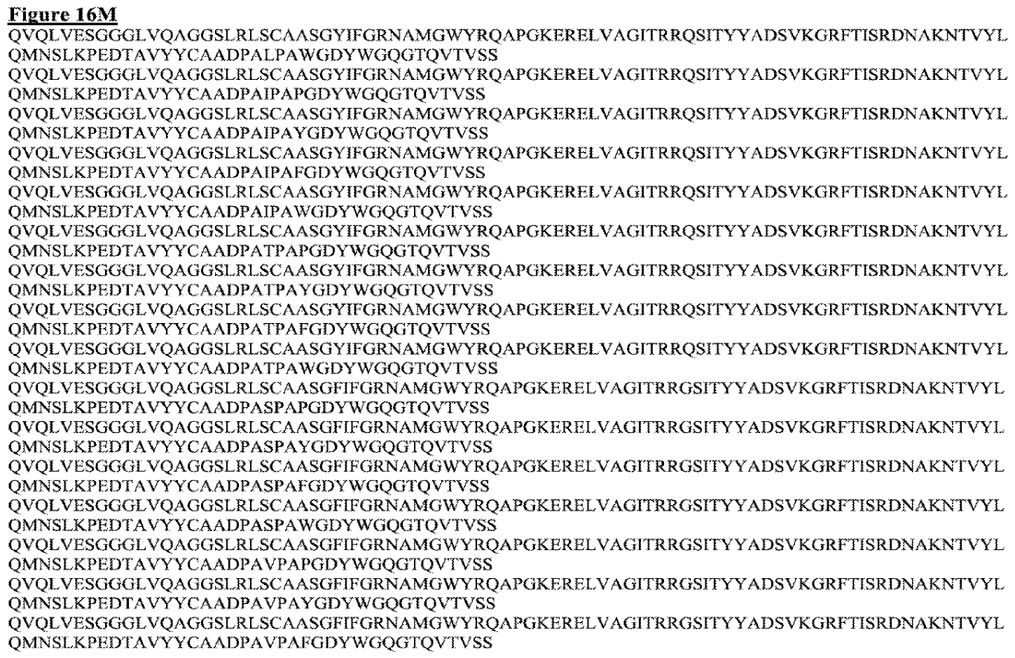

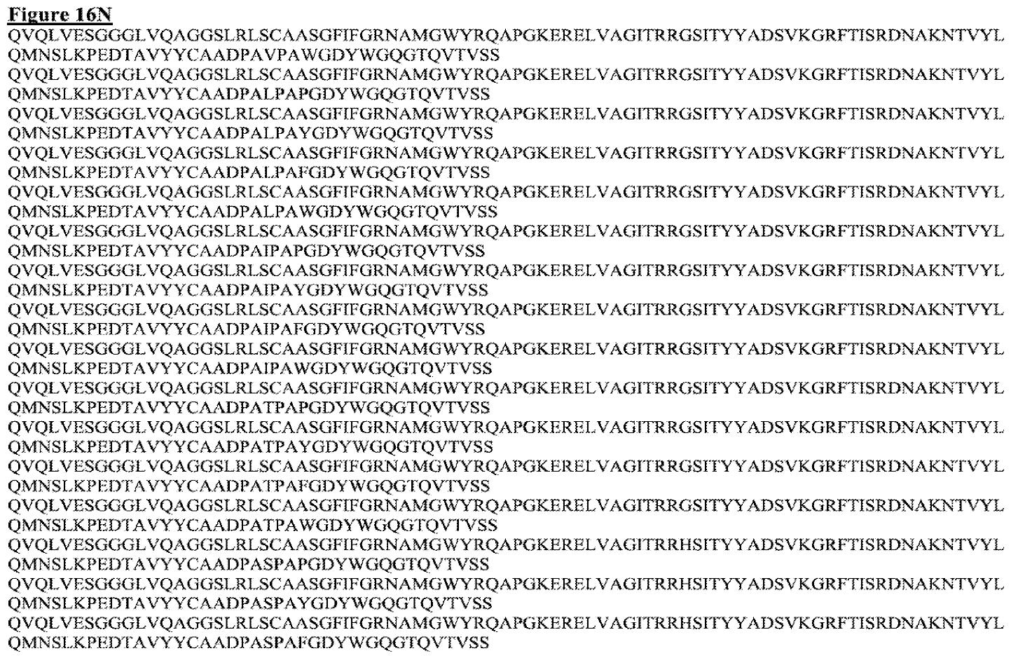

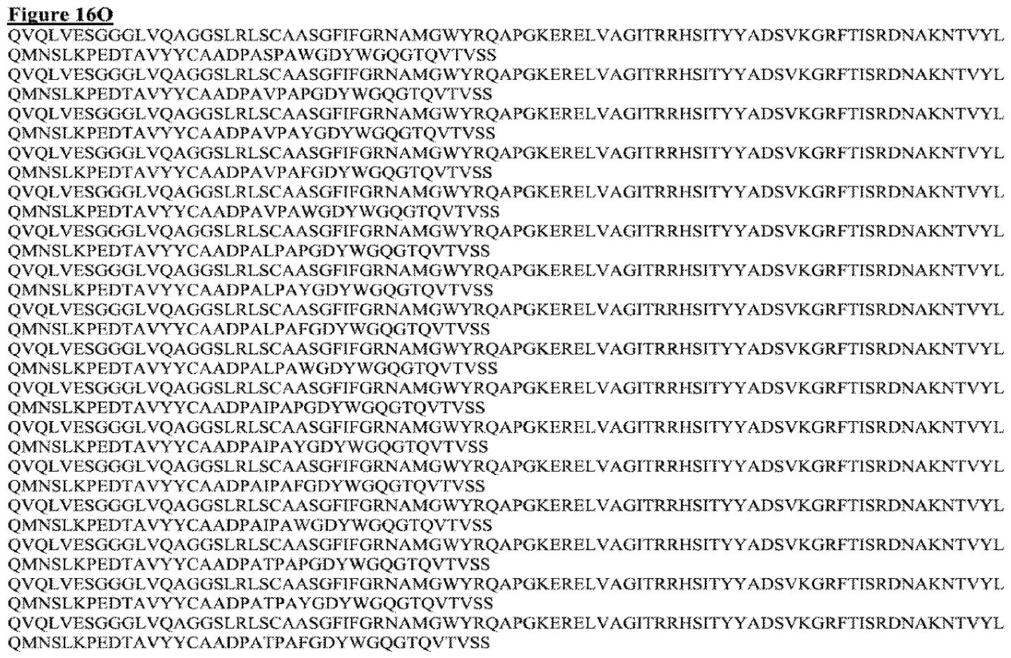

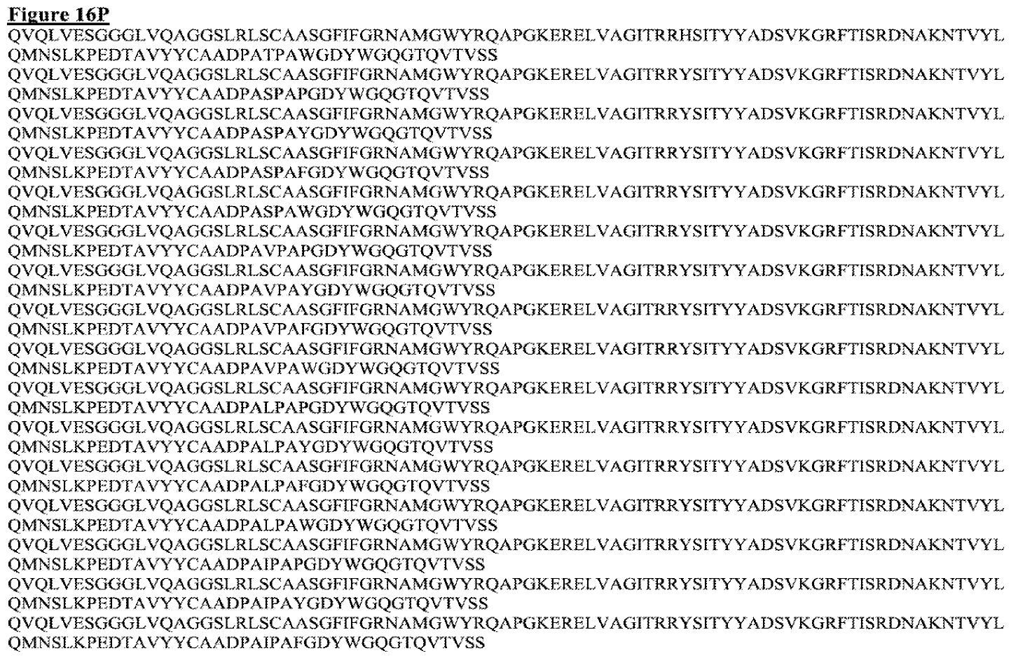

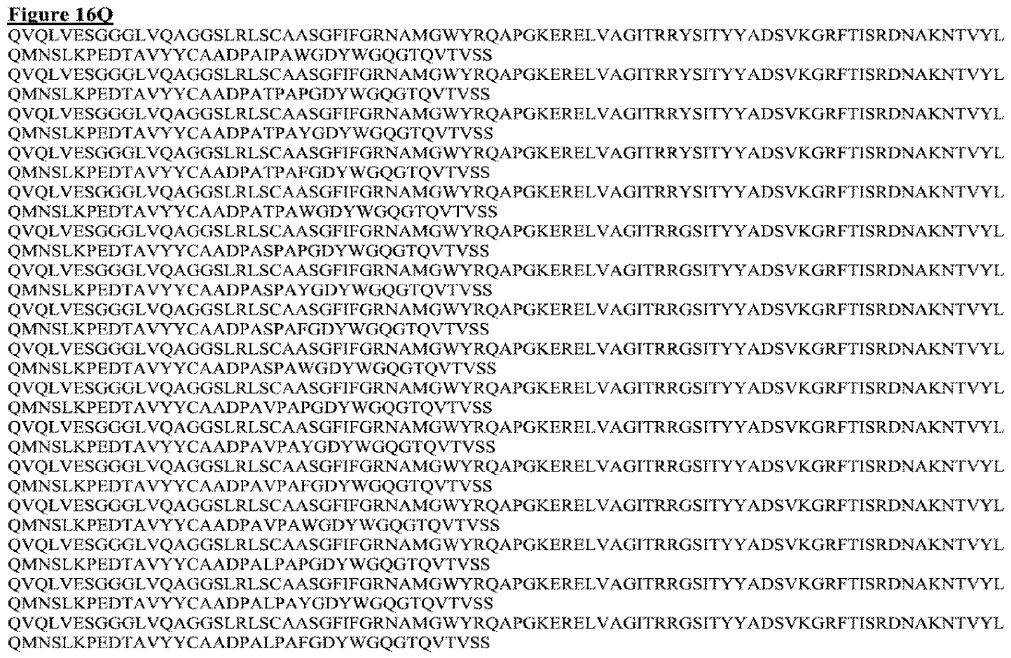

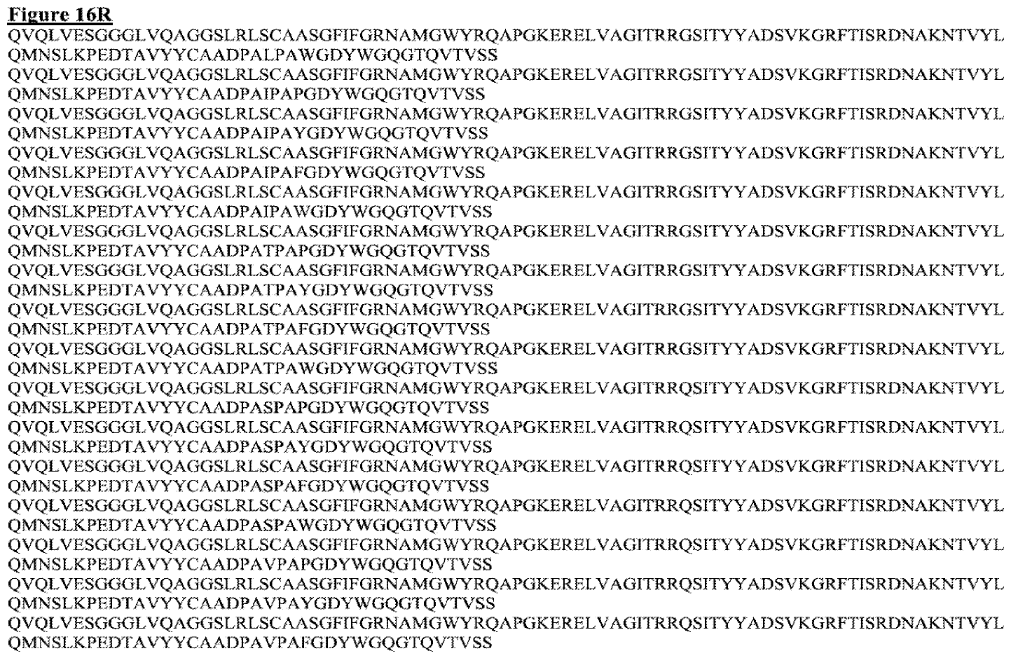

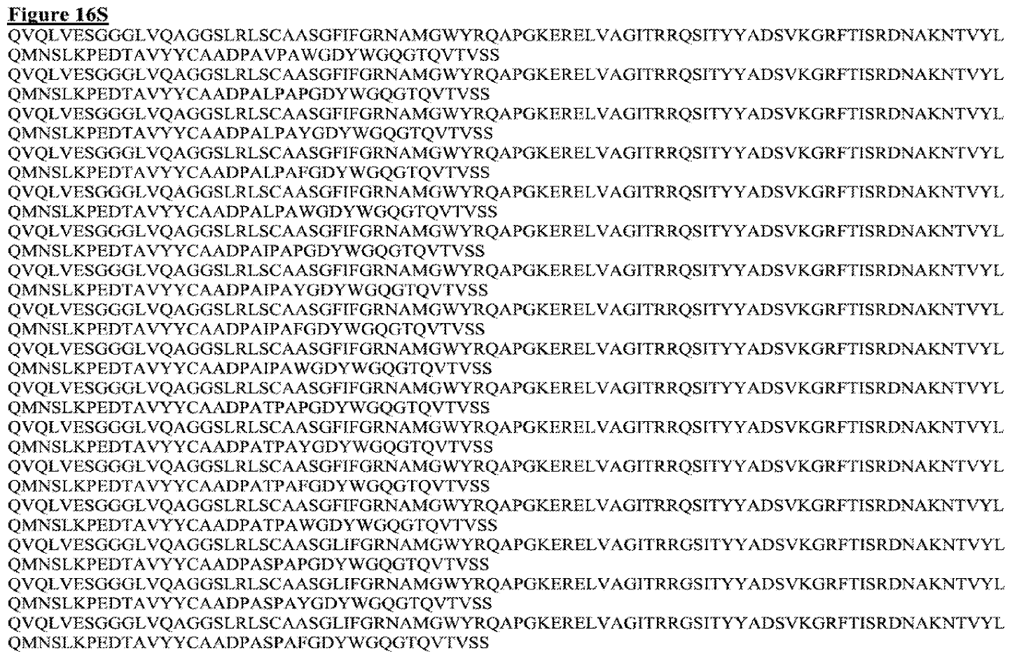

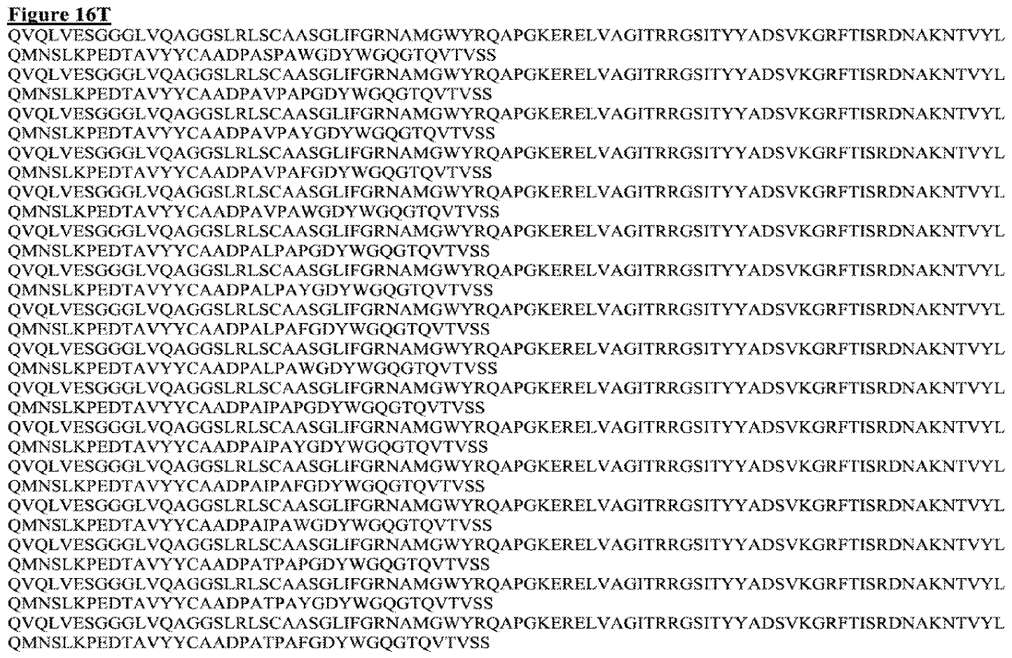

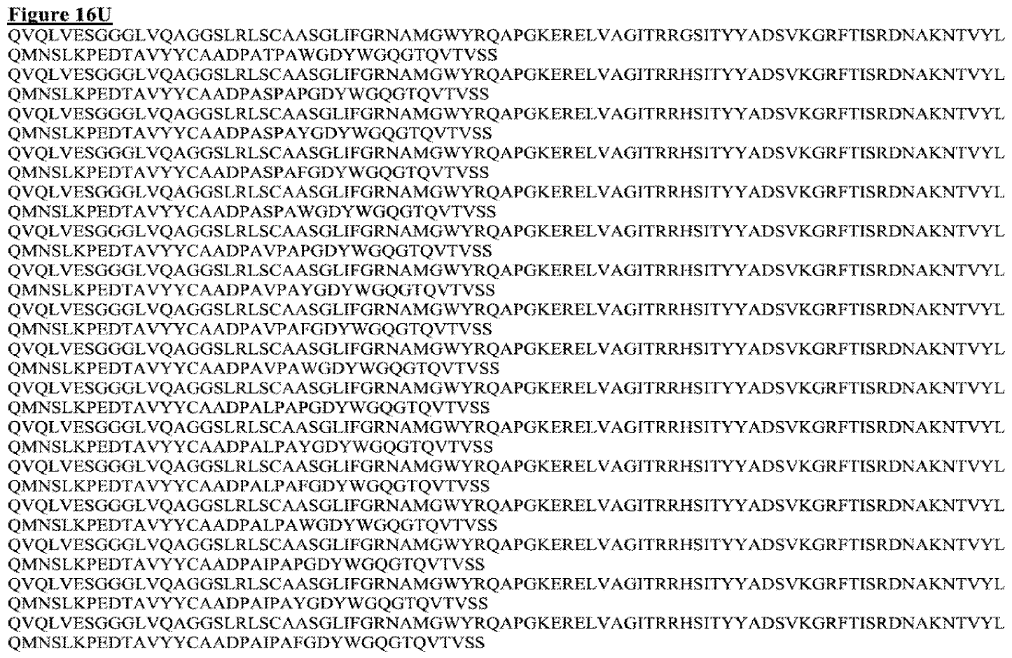

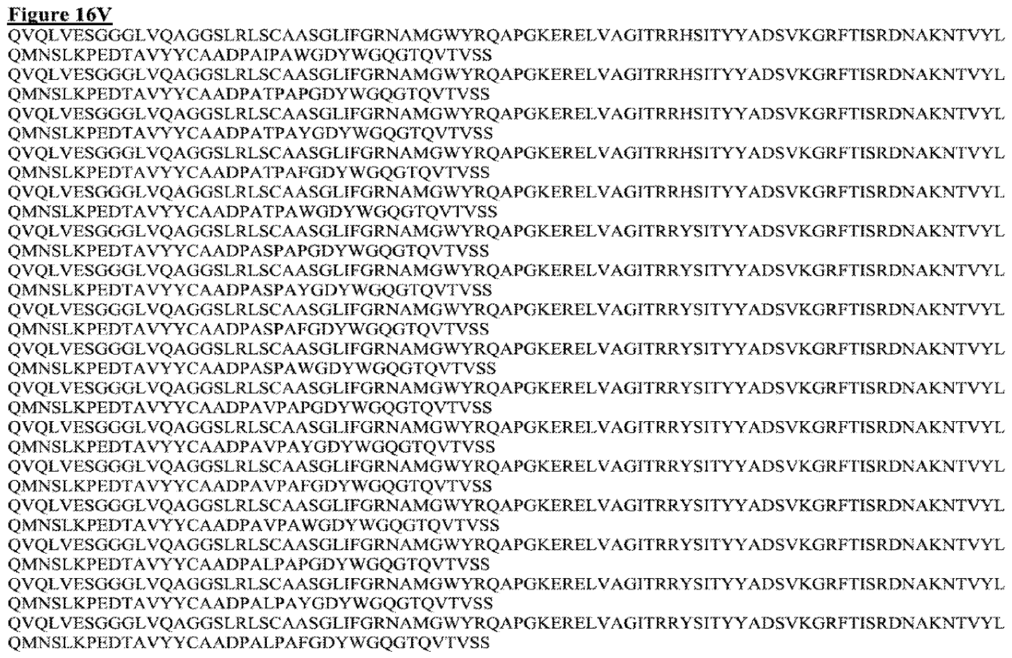

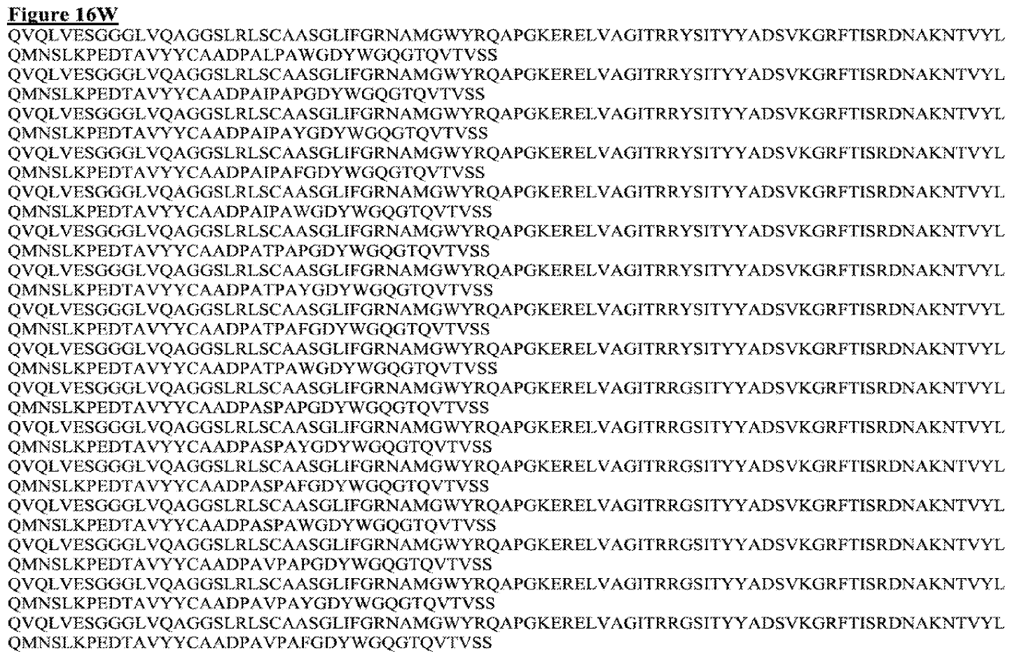

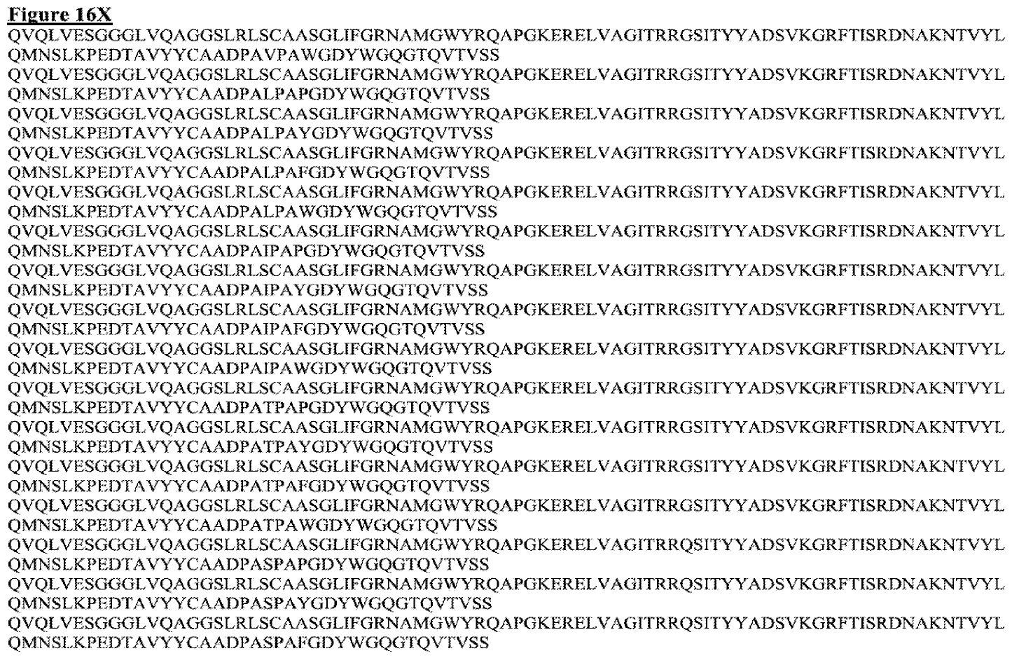

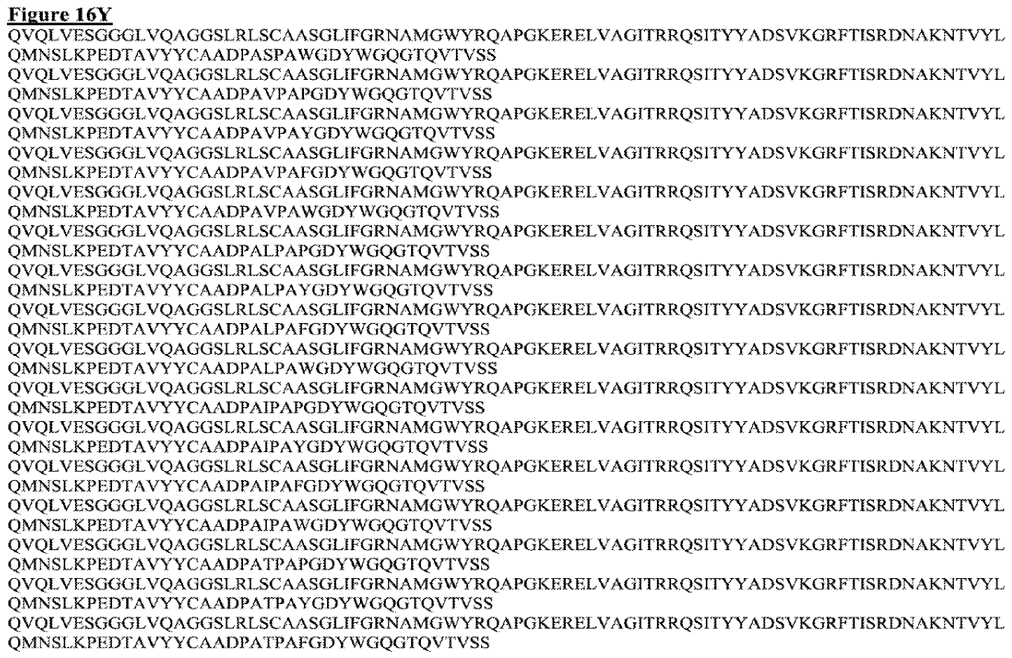

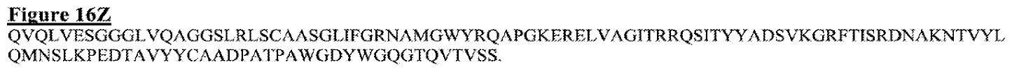

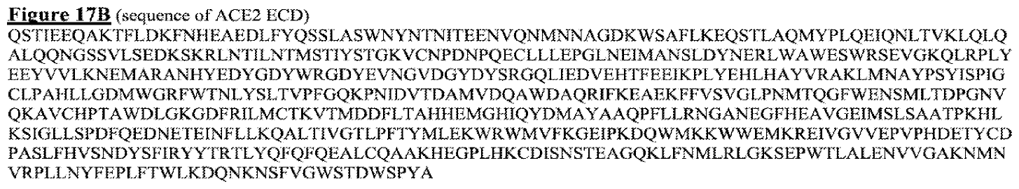

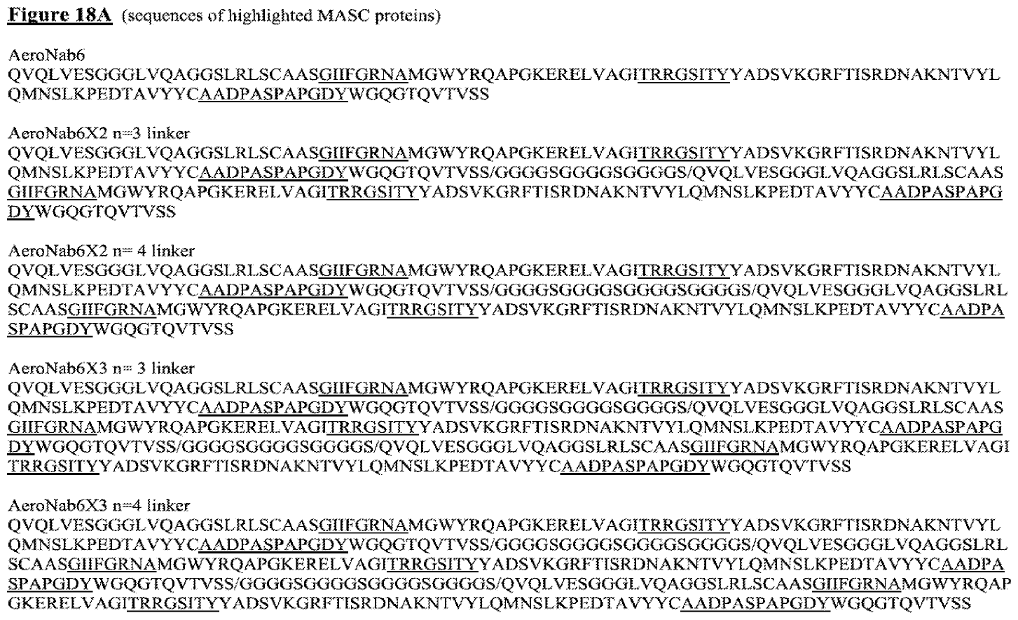

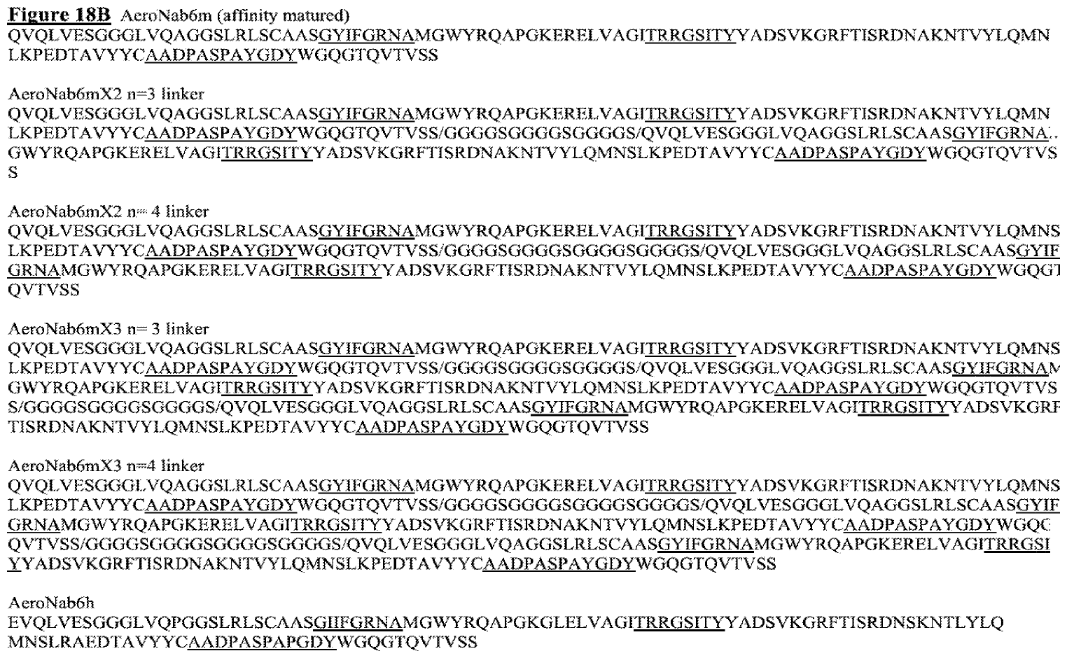

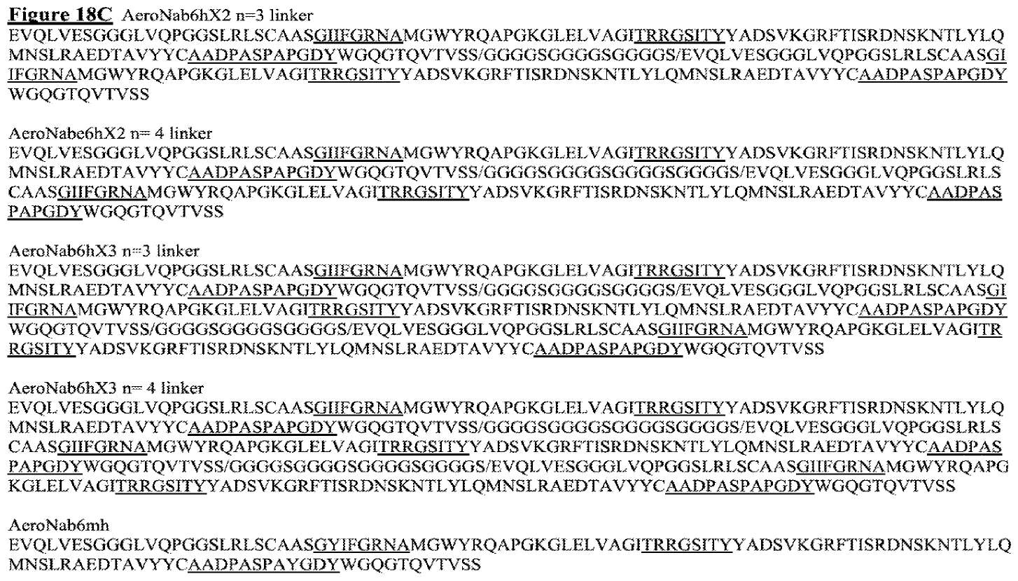

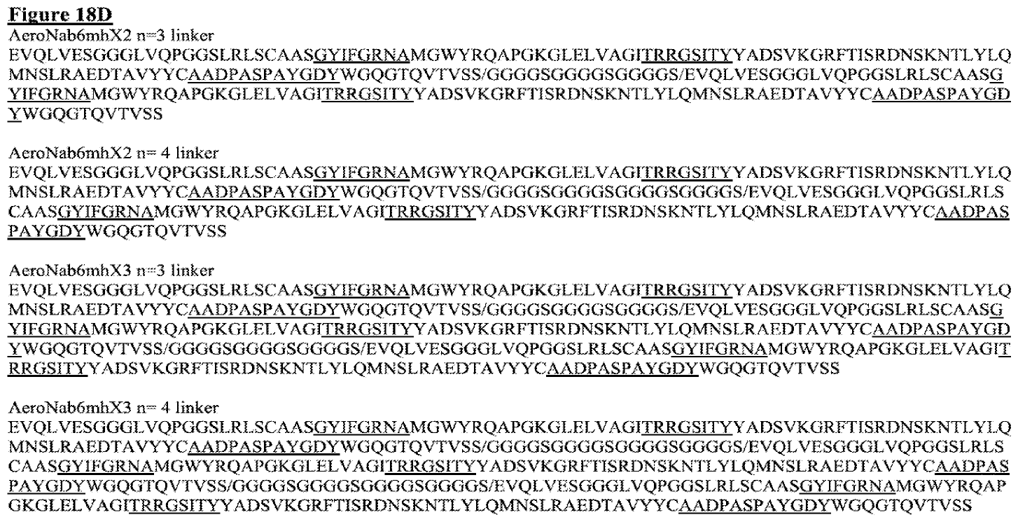

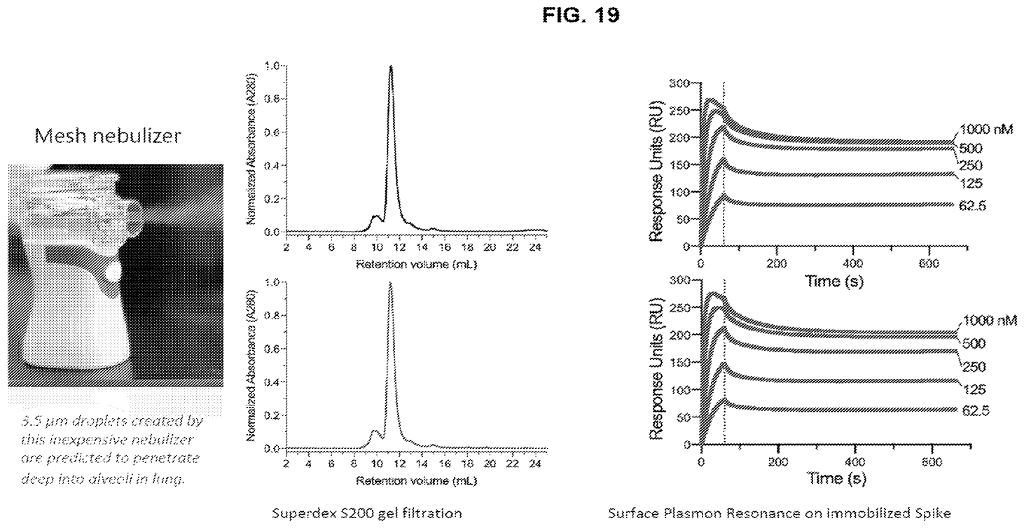

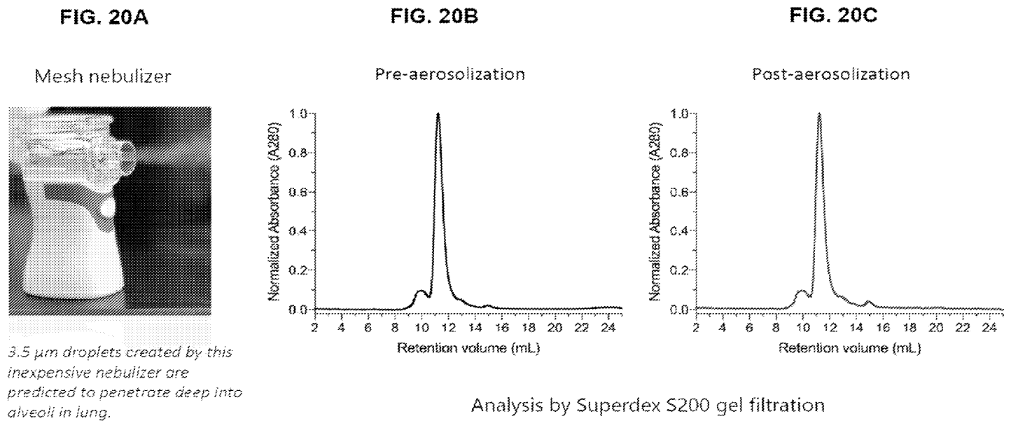

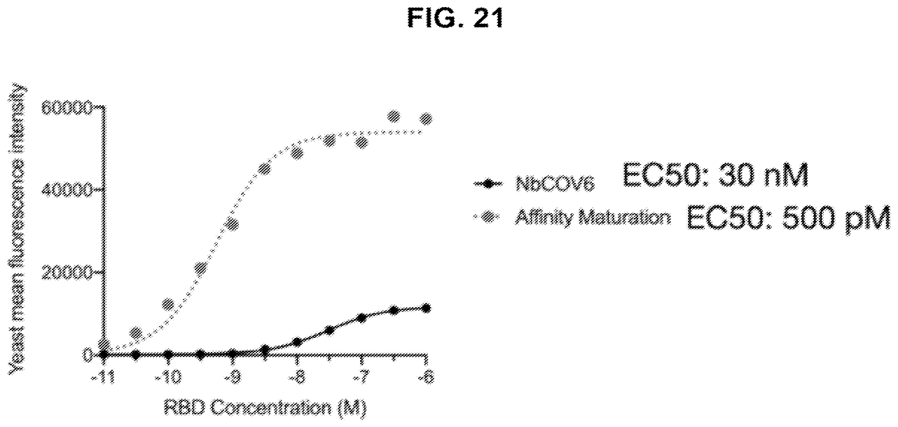

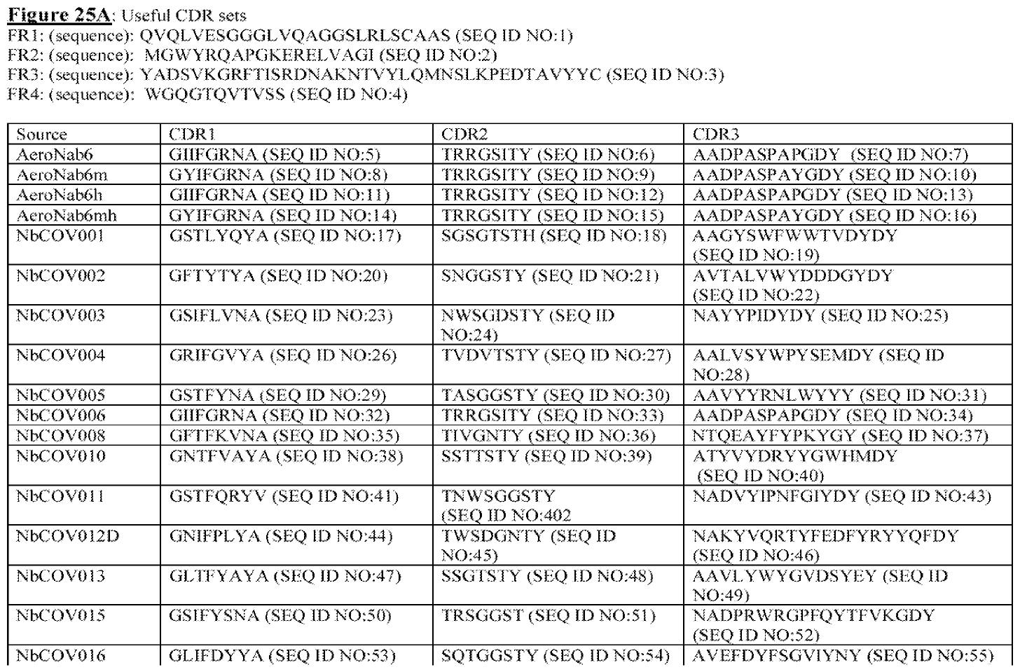

In some embodiments, the MASC protein is a single sdABD, as generally depicted in FIG. 6, and thus is a composition comprising a sdABD comprising, from N- to C-terminal, FR1-vhCDR1-FR2-vhCDR2-FR3-vhCDR3-FR, wherein the vhhCDR1, vhhCDR2 and vhhCDR3 domains are selected from the sets depicted in FIG. 13, FIG. 15, FIG. 18 and FIG. 25.

In some monomeric embodiments, the said vhCDR1 has a sequence GI(I/Y/W/F/V/L)FGRNA, said vhCDR2 has a sequence TRR(G/H/Y/G/Q)SITY and said vhCDR3 has a sequence AADPA(S/V/L/I/T)PA(P/F/W/Y/L/V)GDY as discussed above. Additionally, in this embodiment, the framework regions can have SEQ ID NO:1 (FR1), SEQ ID NO:2 (FR2), SEQ ID NO:3 (FR3) and SEQ ID NO:4 (FR4).

In some embodiments, the sdABD has a set of a three CDRs where vhCDR1 has SEQ ID NO:5, vhCDR2 has SEQ ID NO:6, and vhCDR3 has SEQ ID NO:7. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:8, vhCDR2 has SEQ ID NO:9, and vhCDR3 has SEQ ID NO:10. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:11, vhCDR2 has SEQ ID NO:12, and vhCDR3 has SEQ ID NO:13. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:14, vhCDR2 has SEQ ID NO:15, and vhCDR3 has SEQ ID NO:16. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:17, vhCDR2 has SEQ ID NO:18, and vhCDR3 has SEQ ID NO:19. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:20, vhCDR2 has SEQ ID NO:21, and vhCDR3 has SEQ ID NO:22. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:23, vhCDR2 has SEQ ID NO:24, and vhCDR3 has SEQ ID NO:25. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:26, vhCDR2 has SEQ ID NO:27, and vhCDR3 has SEQ ID NO:28. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:29, vhCDR2 has SEQ ID NO:30, and vhCDR3 has SEQ ID NO:31. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:32, vhCDR2 has SEQ ID NO:33, and vhCDR3 has SEQ ID NO:34. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:35, vhCDR2 has SEQ ID NO:36, and vhCDR3 has SEQ ID NO:37. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:38, vhCDR2 has SEQ ID NO:39, and vhCDR3 has SEQ ID NO:40. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:41, vhCDR2 has SEQ ID NO:42, and vhCDR3 has SEQ ID NO:43. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO44:, vhCDR2 has SEQ ID NO45:, and vhCDR3 has SEQ ID NO:46. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:47, vhCDR2 has SEQ ID NO:48, and vhCDR3 has SEQ ID NO:49. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:50, vhCDR2 has SEQ ID NO:51, and vhCDR3 has SEQ ID NO:52. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:53, vhCDR2 has SEQ ID NO:54, and vhCDR3 has SEQ ID NO:55. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:56, vhCDR2 has SEQ ID NO:57, and vhCDR3 has SEQ ID NO:58. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:59, vhCDR2 has SEQ ID NO:60, and vhCDR3 has SEQ ID NO:61. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID N062 vhCDR2 has SEQ ID NO:63, and vhCDR3 has SEQ ID NO:64. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:65, vhCDR2 has SEQ ID NO:66, and vhCDR3 has SEQ ID NO:67. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:68, vhCDR2 has SEQ ID NO:69, and vhCDR3 has SEQ ID NO:70. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:71, vhCDR2 has SEQ ID NO:72, and vhCDR3 has SEQ ID NO:73. In this embodiment, the framework regions can have SEQ ID NO:1 (FR1), SEQ ID NO:2 (FR2), SEQ ID NO:3 (FR3) and SEQ ID NO:4 (FR4), or can be different.

In some embodiments, the sdABD has a set of a three CDRs where vhCDR1 has SEQ ID NO:74, vhCDR2 has SEQ ID NO:75, and vhCDR3 has SEQ ID NO:76. In this embodiment, the framework regions can have SEQ ID NO:1 (FR1), SEQ ID NO:2 (FR2), SEQ ID NO:3 (FR3) and SEQ ID NO:4 (FR4), or can be different.

In a particularly useful embodiment, the MASC protein is “AeroNab6mh” and has the sequence

EVQLVESGGGLVQPGGSLRLSCAASGYIFGRNAMGWYRQAPGKGLELVA
GITRRGSITYYADSVKGRFTISRDNSKNTLYLQMNSLRAEDTAVYYCAA
DPASPAYGDYWGQGTQVTVSS.

2. Dimeric Constructs

In some embodiments, the MASC protein is a MASC fusion protein and contains two sdABDs, as generally depicted in FIG. 6, and thus is a composition comprising a sdABD comprising, from N- to C-terminal, FR1-vhhCDR1-FR2-vhhCDR2-FR3-vhhCDR3-FR4-domain linker-FR1-vhhCDR1-FR2-vhhCDR2-FR3-vhhCDR3-FR4, wherein the vhhCDR1, vhhCDR2 and vhhCDR3 domains are selected from the sets depicted in FIG. 13, FIG. 15, FIG. 18 and FIG. 25.

In many embodiments, the two sdABDs that make up the dimer are the same, and the domain linker is selected from (GGGGS)₃ and (GGGGS)₄.

Accordingly, in some dimeric embodiments, the said vhCDR1 has a sequence GI(I/Y/W/F/V/L)FGRNA, said vhCDR2 has a sequence TRR(G/H/Y/G/Q)SITY and said vhCDR3 has a sequence AADPA(S/V/L/I/T)PA(P/F/W/Y/L/V)GDY as discussed above. Additionally, in this embodiment, the framework regions can have SEQ ID NO:1 (FR1), SEQ ID NO:2 (FR2), SEQ ID NO:3 (FR3) and SEQ ID NO:4 (FR4) and the domain linker is (GGGGS)₃.

Accordingly, in some dimeric embodiments, the said vhCDR1 has a sequence GI(I/Y/W/F/V/L)FGRNA, said vhCDR2 has a sequence TRR(G/H/Y/G/Q)SITY and said vhCDR3 has a sequence AADPA(S/V/L/I/T)PA(P/F/W/Y/L/V)GDY as discussed above. Additionally, in this embodiment, the framework regions can have SEQ ID NO:1 (FR1), SEQ ID NO:2 (FR2), SEQ ID NO:3 (FR3) and SEQ ID NO:4 (FR4) and the domain linker is (GGGGS)_{4.}

In some dimeric embodiments, the two sdABDs each have a set of three CDRs where vhCDR1 has SEQ ID NO:5, vhCDR2 has SEQ ID NO:6, and vhCDR3 has SEQ ID NO:7. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:8, vhCDR2 has SEQ ID NO:9, and vhCDR3 has SEQ ID NO:10. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:11, vhCDR2 has SEQ ID NO:12, and vhCDR3 has SEQ ID NO:13. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:14, vhCDR2 has SEQ ID NO:15, and vhCDR3 has SEQ ID NO:16. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄. In some dimeric embodiments, the two sdABDs each have a set of three CDRs where vhCDR1 has SEQ ID NO:17, vhCDR2 has SEQ ID NO:18, and vhCDR3 has SEQ ID NO:19. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:20, vhCDR2 has SEQ ID NO:21, and vhCDR3 has SEQ ID NO:22. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:23, vhCDR2 has SEQ ID NO:24, and vhCDR3 has SEQ ID NO:25. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:26, vhCDR2 has SEQ ID NO:27, and vhCDR3 has SEQ ID NO:28. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:29, vhCDR2 has SEQ ID NO:30, and vhCDR3 has SEQ ID NO:31. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:32, vhCDR2 has SEQ ID NO:33, and vhCDR3 has SEQ ID NO:34. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:35, vhCDR2 has SEQ ID NO:36, and vhCDR3 has SEQ ID NO:37. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:38, vhCDR2 has SEQ ID NO:39, and vhCDR3 has SEQ ID NO:40. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:41, vhCDR2 has SEQ ID NO:42, and vhCDR3 has SEQ ID NO:43. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO44:, vhCDR2 has SEQ ID NO45:, and vhCDR3 has SEQ ID NO:46. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:47, vhCDR2 has SEQ ID NO:48, and vhCDR3 has SEQ ID NO:49. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:50, vhCDR2 has SEQ ID NO:51, and vhCDR3 has SEQ ID NO:52. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:53, vhCDR2 has SEQ ID NO:54, and vhCDR3 has SEQ ID NO:55. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:56, vhCDR2 has SEQ ID NO:57, and vhCDR3 has SEQ ID NO:58. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:59, vhCDR2 has SEQ ID NO:60, and vhCDR3 has SEQ ID NO:61. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID N062 vhCDR2 has SEQ ID NO:63, and vhCDR3 has SEQ ID NO:64. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:65, vhCDR2 has SEQ ID NO:66, and vhCDR3 has SEQ ID NO:67. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:68, vhCDR2 has SEQ ID NO:69, and vhCDR3 has SEQ ID NO:70. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:71, vhCDR2 has SEQ ID NO:72, and vhCDR3 has SEQ ID NO:73. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some dimeric embodiments, the two sdABDs each have a set of three CDRs where vhCDR1 has SEQ ID NO:74, vhCDR2 has SEQ ID NO:75, and vhCDR3 has SEQ ID NO:76. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In a particularly useful embodiment, the MASC protein is “AeroNab6mhX2” and has the sequence:

EVQLVESGGGLVQPGGSLRLSCAASGYIFGRNAMGWYRQAPGKGLELVA
GITRRGSITYYADSVKGRFTISRDNSKNTLYLQMNSLRAEDTAVYYCAA
DPASPAYGDYWGQGTQVTVSS/GGGGSGGGGSGGGGS/EVQLVESGGGL
VQPGGSLRLSCAASGYIFGRNAMGWYRQAPGKGLELVAGITRRGSITYY
ADSVKGRFTISRDNSKNTLYLQMNSLRAEDTAVYYCAADPASPAYGDYW
GQGTQVTVSS

In a particularly useful embodiment, the MASC protein is “AeroNab6mhX2” and has the sequence:

EVQLVESGGGLVQPGGSLRLSCAASGYIFGRNAMGWYRQAPGKGLELVA
GITRRGSITYYADSVKGRFTISRDNSKNTLYLQMNSLRAEDTAVYYCAA
DPASPAYGDYWGQGTQVTVSS/GGGGSGGGGSGGGGSGGGGS/EVQLVE
SGGGLVQPGGSLRLSCAASGYIFGRNAMGWYRQAPGKGLELVAGITRRG
SITYYADSVKGRFTISRDNSKNTLYLQMNSLRAEDTAVYYCAADPASPA
YGDYWGQGTQVTVSS

In some embodiments, the two sdABDs that make up the dimer are different. For example, in one embodiment, one of the sdABDs is “AeroNab6mh” and the other has the CDRs of NbCoV003, SEQ ID NO:21, SEQ ID NO:22 and SEQ ID NO:23; see FIG. 26.

3. Trimeric Constructs

In some embodiments, the MASC protein is a MASC fusion protein and contains three sdABDs, as generally depicted in FIG. 6, and thus is a composition comprising a sdABD comprising, from N- to C-terminal, FR1-vhhCDR1-FR2-vhhCDR2-FR3-vhhCDR3-FR4-domain linker-FR1-vhhCDR1-FR2-vhhCDR2-FR3-vhhCDR3-FR4, wherein the vhhCDR1, vhhCDR2 and vhhCDR3 domains are selected from the sets depicted in FIG. 13, FIG. 15, FIG. 18 and FIG. 25.

In many embodiments, the three sdABDs that make up the trimer are the same, and the domain linker is selected from (GGGGS)₃ and (GGGGS)₄.

Accordingly, in some trimeric embodiments, the said vhCDR1 has a sequence GI(I/Y/W/F/V/L)FGRNA, said vhCDR2 has a sequence TRR(G/H/Y/G/Q)SITY and said vhCDR3 has a sequence AADPA(S/V/L/I/T)PA(P/F/W/Y/L/V)GDY as discussed above. Additionally, in this embodiment, the framework regions can have SEQ ID NO:1 (FR1), SEQ ID NO:2 (FR2), SEQ ID NO:3 (FR3) and SEQ ID NO:4 (FR4) and the domain linker is (GGGGS)₃.

Accordingly, in some trimeric embodiments, the said vhCDR1 has a sequence GI(I/Y/W/F/V/L)FGRNA, said vhCDR2 has a sequence TRR(G/H/Y/G/Q)SITY and said vhCDR3 has a sequence AADPA(S/V/L/I/T)PA(P/F/W/Y/L/V)GDY as discussed above. Additionally, in this embodiment, the framework regions can have SEQ ID NO:1 (FR1), SEQ ID NO:2 (FR2), SEQ ID NO:3 (FR3) and SEQ ID NO:4 (FR4) and the domain linker is (GGGGS)₄.

In some trimeric embodiments, the two sdABDs each have a set of three CDRs where vhCDR1 has SEQ ID NO:5, vhCDR2 has SEQ ID NO:6, and vhCDR3 has SEQ ID NO:7. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:8, vhCDR2 has SEQ ID NO:9, and vhCDR3 has SEQ ID NO:10. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:11, vhCDR2 has SEQ ID NO:12, and vhCDR3 has SEQ ID NO:13. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:14, vhCDR2 has SEQ ID NO:15, and vhCDR3 has SEQ ID NO:16. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric dimeric embodiments, the two sdABDs each have a set of three CDRs where vhCDR1 has SEQ ID NO:17, vhCDR2 has SEQ ID NO:18, and vhCDR3 has SEQ ID NO:19. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:20, vhCDR2 has SEQ ID NO:21, and vhCDR3 has SEQ ID NO:22. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:23, vhCDR2 has SEQ ID NO:24, and vhCDR3 has SEQ ID NO:25. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:26, vhCDR2 has SEQ ID NO:27, and vhCDR3 has SEQ ID NO:28. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:29, vhCDR2 has SEQ ID NO:30, and vhCDR3 has SEQ ID NO:31. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:32, vhCDR2 has SEQ ID NO:33, and vhCDR3 has SEQ ID NO:34. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:35, vhCDR2 has SEQ ID NO:36, and vhCDR3 has SEQ ID NO:37. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:38, vhCDR2 has SEQ ID NO:39, and vhCDR3 has SEQ ID NO:40. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:41, vhCDR2 has SEQ ID NO:42, and vhCDR3 has SEQ ID NO:43. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO44:, vhCDR2 has SEQ ID NO45:, and vhCDR3 has SEQ ID NO:46. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:47, vhCDR2 has SEQ ID NO:48, and vhCDR3 has SEQ ID NO:49. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:50, vhCDR2 has SEQ ID NO:51, and vhCDR3 has SEQ ID NO:52. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:53, vhCDR2 has SEQ ID NO:54, and vhCDR3 has SEQ ID NO:55. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:56, vhCDR2 has SEQ ID NO:57, and vhCDR3 has SEQ ID NO:58. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:59, vhCDR2 has SEQ ID NO:60, and vhCDR3 has SEQ ID NO:61. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID N062 vhCDR2 has SEQ ID NO:63, and vhCDR3 has SEQ ID NO:64. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:65, vhCDR2 has SEQ ID NO:66, and vhCDR3 has SEQ ID NO:67. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:68, vhCDR2 has SEQ ID NO:69, and vhCDR3 has SEQ ID NO:70. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:71, vhCDR2 has SEQ ID NO:72, and vhCDR3 has SEQ ID NO:73. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In some trimeric embodiments, the two sdABDs each have a set of three CDRs where vhCDR1 has SEQ ID NO:74, vhCDR2 has SEQ ID NO:75, and vhCDR3 has SEQ ID NO:76. In this embodiment, the framework regions can have SEQ ID NO:1 (FR1), SEQ ID NO:2 (FR2), SEQ ID NO:3 (FR3) and SEQ ID NO:4 (FR4), and the domain linker is selected from (GGGGS)₃ and (GGGGS)₄.

In a particularly useful embodiment, the MASC protein is “AeroNab6mhX3” and has the sequence:

EVQLVESGGGLVQPGGSLRLSCAASGYIFGRNAMGWYRQAPGKGLELVA
GITRRGSITYYADSVKGRFTISRDNSKNTLYLQMNSLRAEDTAVYYCAA
DPASPAYGDYWGQGTQVTVSS/GGGGSGGGGSGGGGS/EVQLVESGGGL
VQPGGSLRLSCAASGYIFGRNAMGWYRQAPGKGLELVAGITRRGSITYY
ADSVKGRFTISRDNSKNTLYLQMNSLRAEDTAVYYCAADPASPAYGDYW
GQGTQVTVSS/GGGGSGGGGSGGGGS/EVQLVESGGGLVQPGGSLRLSC
AASGYIFGRNAMGWYRQAPGKGLELVAGITRRGSITYYADSVKGRFTIS
RDNSKNTLYLQMNSLRAEDTAVYYCAADPASPAYGDYWGQGTQVTVSS

In a particularly useful embodiment, the MASC protein is “AeroNab6mhX3” and has the sequence:

EVQLVESGGGLVQPGGSLRLSCAASGYIFGRNAMGWYRQAPGKGLELVA
GITRRGSITYYADSVKGRFTISRDNSKNTLYLQMNSLRAEDTAVYYCAA
DPASPAYGDYWGQGTQVTVSS/GGGGSGGGGSGGGGSGGGGS/EVQLVE
SGGGLVQPGGSLRLSCAASGYIFGRNAMGWYRQAPGKGLELVAGITRRG
SITYYADSVKGRFTISRDNSKNTLYLQMNSLRAEDTAVYYCAADPASPA
YGDYWGQGTQVTVSS/GGGGSGGGGSGGGGSGGGGS/EVQLVESGGGLV
QPGGSLRLSCAASGYIFGRNAMGWYRQAPGKGLELVAGITRRGSITYYA
DSVKGRFTISRDNSKNTLYLQMNSLRAEDTAVYYCAADPASPAYGDYWG
QGTQVTVSS

In some embodiments, the two sdABDs that make up the dimer are different. For example, in one embodiment, one of the sdABDs is “AeroNab6mh” and the other has the CDRs of NbCoV003, SEQ ID NO:21, SEQ ID NO:22 and SEQ ID NO:23.

4. Domain Linkers

In embodiments utilizing multimeric MASC proteins, the monomers are linked recombinantly using “domain linkers”. While any suitable linker can be used, many embodiments utilize a glycine-serine polymer, including for example (GS)n, (GSGGS)n, (GGGGS)n, and (GGGS)n, where n is an integer of at least one (and generally from 3 to 4 to 5) as well as any peptide sequence that allows for recombinant attachment of the two domains with sufficient length and flexibility to allow each domain to retain its biological function. As shown in FIG. 4, the distance between the N- and C-termini of individual AeroNab6 monomers bound to spike ECD in the “down” state is 51 Å. This requires ≥15 amino acids to bridge individual subunits to simultaneously engage multiple RBD monomers. Thus, particularly preferred are (GGGGS)₃ and (GGGGS)₄ linkers. As shown in FIG. 23 both (GGGGS)₃ and (GGGGS)₄ linkers in the trimeric constructs show both good binding to the spike protein trimer as measured by SPR.

5. Half-Life Extension Domains

The MASC proteins optionally include half-life extension domains, that allow for increased half-life in physiological environments such as plasma and lung tissue. Such domains are contemplated to include, but are not limited to, HSA binding domains, either scFvs or sdABDs, as well as all or part of human serum albumin, as discussed below.

Human serum albumin (HSA) (molecular mass ˜67 kDa) is the most abundant protein in plasma, present at about 50 mg/ml (600 uM), and has a half-life of around 20 days in humans. HSA serves to maintain plasma pH, contributes to colloidal blood pressure, functions as carrier of many metabolites and fatty acids, and serves as a major drug transport protein in plasma.

Noncovalent association with albumin extends the elimination half-time of short lived proteins. For example, a recombinant fusion of an albumin binding domain to a Fab fragment resulted in a reduced in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In one aspect, the antigen-binding proteins described herein comprise a half-life extension domain, for example a domain which specifically binds to HSA, that is attached either N- or C-terminal to the MASC protein. In many embodiments, the half-life extension domain is a single domain antigen binding domain from a single domain antibody that binds to HSA. This domain is generally referred to herein as “sdABD” to human HSA (sdABD-HSA), or alternatively “sdABD(½)”, to distinguish these binding domains from the sdABDs to the spike protein. Suitable sdABD-HSA domains are well known in the art, see for example U.S. Pat. No. 8,703,131, the sequences of all sdABD-HSA domains therein (“ALB”, including specifically ALB1, ALB3, ALB4, ALB5, ALB6, ALB7, ALB8, ALB9 and ALB10) are expressly incorporated by reference. Similarly, U.S. Pat. No. 10,100,106 contains additional single domain albumin binding domains, the sequences of which are also specifically incorporated by reference herein, including SEQ ID NOs:4, 7, 9, 26 and 27.

Another suitable half-life domain that can be fused to the MASC proteins is all or part of human HSA itself, again, either N- or C-terminal. HSA is a relatively small protein, roughly 65 amino acids long, and can be fused to one or more of the monomeric MASC proteins as will be appreciated by those in the art.

The half-life extension domain of an antigen binding protein provides for altered pharmacodynamics and pharmacokinetics of the MASC proteins. As above, the half-life extension domain extends the elimination half-time. The half-life extension domain also alters pharmacodynamic properties including alteration of tissue distribution, penetration, and diffusion of the antigen-binding protein.

D. Method of Making MASC Proteins

The MASC proteins and fusion proteins of the invention are made as will generally be appreciated by those in the art and outlined below.

The invention provides nucleic acid compositions that encode the MASC compositions of the invention. As is known in the art, the nucleic acids encoding the compositions of the invention can be incorporated into expression vectors as is known in the art, and depending on the host cells used to produce the MASC proteins of the invention. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the invention are then transformed into any number of different types of host cells as is well known in the art, including mammalian, bacterial, yeast, insect and/or fungal cells, with mammalian cells (e.g. CHO cells, 293 cells), finding use in many embodiments.

The MASC proteins, including MASC fusion proteins, of the invention are made by culturing host cells comprising the expression vector(s) as is well known in the art under conditions that result in the expression of the proteins, followed by purification.

E. Formulations

Formulations of the MASC proteins used in accordance with the present invention are prepared for storage by mixing the proteins having the desired degree of purity with optional pharmaceutically acceptable carriers, excipients or stabilizers (as generally outlined in Remington's Pharmaceutical Sciences 16th edition, Osol, A. Ed. [1980]), in the form of lyophilized formulations or aqueous solutions.

F. Administration of the MASC Proteins

The compositions of the invention comprising MASC proteins and MASC fusion proteins that are administered to a patient to prevent, treat or neutralize the SC2 virus or SC2 viral infection in a patient. As recently reported, it appears that the highest ACE2 expression in humans is in the nose with decreasing expression throughout the lower respiratory tract, corresponding to a gradient of SC2 infection from high in the proximal nasal passages to lower in the distal areas such as peripheral lung (see Hou et al, https://doi.org/10.1016/j.cell.2020.05.042), supporting an early hypothesis that nasal surfaces might be the dominant initial site for infection. The infection then proceeds to the lungs.

Accordingly, as will be appreciated by the art, there are a number of different administration routes which can be utilized to administer the MASC proteins of the invention, including, but not limited to, pulmonary delivery using inhalation techniques, intranasal delivery using particular formulations and intravenous administration, as outlined herein.

1. Inhalation Therapy

Thus, in some embodiments, the MASC proteins are administered to a patient's pulmonary system, including the lungs. Thus, the invention provides for the delivery of the MASC proteins (including MASC fusion proteins) of the invention to the respiratory tract.

One benefit of the MASC proteins of the invention is that they are extremely stable and thus can be lyophilized, as is known in the art. The lyophilized proteins can then be reconstituted at a later date into a liquid formulation and then aerosolized through nebulization for direct delivery to the patient's pulmonary system. See for example U.S. Pat. Nos. 9,393,304 which describes a number of lyophilization techniques, conditions and formulations for Nanobodies™ that are for inhalation therapy.

In certain embodiments, the formulations can be administered using nebulizers. Examples of nebulizers include, in non-limiting examples, jet nebulizers, ultrasonic nebulizers, and vibrating mesh nebulizers. These classes use different methods to create an aerosol from a liquid. In general, any aerosol-generating device that can maintain the integrity of the protein in these formulations is suitable for delivery of formulations as described herein.

In some embodiments, a vibrating-mesh nebulizers is used. Vibrating-mesh nebulizers are divided into passively and actively vibrating-mesh devices (Newman 2005, J. Appl. Ther. Res. 5: 29-33). Passively vibrating-mesh devices (e.g. Omron MICROAIR.®. NE-U22 nebulizer) employ a perforated plate having up to 6000 micron sized holes. A vibrating piezo-electric crystal attached to a transducer horn induces “passive” vibrations in the perforated plate positioned in front of it, resulting in extrusion of fluid through the holes and generation of the aerosol. Actively vibrating-mesh devices (e.g. AERONEB.®. Pro nebulizer) may employ a “micropump” system which comprises an aerosol generator consisting of a plate with up to 1000 dome-shaped apertures and a vibrating element which contracts and expands on application of an electric current. This results in upward and downward movements of the mesh by a few micrometers, extruding the fluid and generating the aerosol. Other examples of vibrating-mesh nebulizers include the Akita2 Apixneb (Activaero, now Vectura, Germany), EFLOW.®. (PARI GmbH, Grafelingen, Germany; see also U.S. Pat. No. 5,586,550), AERONEB.®. (Aerogen, Inc., Sunnyvale, Calif.; see also U.S. Pat. Nos. 5,586,550; 5,938,117; 6,014,970; 6,085,740; 6,205,999), or the FOX nebulizer (Activaero, now Vectura, Germany), all adapted for pediatric use.

In some embodiments, a continuous flow nebuliser is used, particularly in cases where COVID19 patients may require oxygen as well, so continuous flow can be used to maintain a continuous oxygen or air supply to the patient. Accordingly, the nebulizer can be used with or without additional air or 02 flow. Preferably, the nebulizer is used with additional air or O₂ flow, such as a flow of 2 L/min additional air or O₂.

An exemplary inhalation device for delivering the polypeptide of the invention to a patient may comprises (a) an aerosol generator with a vibratable mesh; (b) a reservoir for a liquid to be nebulised, said reservoir being in fluid connection with the vibratable mesh; (c) a gas inlet opening; (d) a face mask, having a casing, an aerosol inlet opening, a patient contacting surface, and a one-way exhalation valve or a two-way inhalation/exhalation valve in the casing having an exhalation resistance selected in the range from 0.5 to 5 mbar; and (e) a flow channel extending from the gas inlet opening to the aerosol inlet opening of the face mask, the flow channel having a lateral opening through which the aerosol generator is at least partially inserted into the flow channel, and a constant flow resistance between the gas inlet opening and the aerosol inlet opening of the face mask at a flow rate of 1 to 20 L/min.

Additional methods for delivery to the respiratory tract and/or delivery by inhalation are known to the skilled person and are e.g. described in the handbook “Drug Delivery: Principles and Applications” (2005) by Binghe Wang, Teruna Siahaan and Richard Soltero (Eds. Wiley Interscience (John Wiley & Sons)); in “Pharmacology PreTest.™. (11.sup.th Ed.) Self-Assessment and Review” by Rosenfeld G. C., Loose-Mitchell D. S.; and in “Pharmacology” (3.sup.rd Edition) by Lippincott Williams & Wilkins, New York; Shlafer M. McGraw-Hill Medical Publishing Division, New York; Yang K. Y., Graff L. R., Caughey A. B. Blueprints Pharmacology, Blackwell Publishing.

The present invention also relates to a pharmaceutical device suitable for the delivery by inhalation of the MASC proteins of the invention and suitable in the use of a composition comprising the same. The present invention, accordingly, relates to such a device comprising the MASC proteins of the invention at the selected dose.

Various inhalation systems are e.g. described on pages 129 to 148 in the review (“Pulmonary Drug Delivery”, Bechtold-Peters and Luessen, eds., supra). In the method of the present invention, the device is an inhaler for liquids (e.g. a suspension of fine solid particles or droplets) comprising the polypeptide of the invention. Preferably this device is an aerosol delivery system or a nebulizer comprising the polypeptide of the invention.

The aerosol delivery system used in the method of the invention may comprise a container comprising the composition of the invention and an aerosol generator connected to it. The aerosol generator is constructed and arranged to generate an aerosol of the composition of the invention.

2. Intranasal Adminstration

As discussed above, it appears that the nasal cavity and nasal surfaces might be the dominant initial site for SC2 viral infection, as evidenced by the high ACE2 expression patterns.

Accordingly, in some embodiments the MASC proteins, including MASC fusion proteins, are administered via nasal administration as a nasal spray. There are a wide variety of delivery systems for intranasal administration of the MASC proteins, ranging from simple drops or sprays to unit dosing systems for liquids; see for example Marx et al., Intranasal Drug Administration—An Attractive Delivery Route for Some Drugs; DOI: 10.5772/59468. As above, the MASC proteins can be lyophilized and then reconstituted for nasal administration or administered directly as a liquid with lyophilization.

3. Intravenous Administration

Additionally, as will be appreciated by those in the art, the MASC proteins of the invention can also be administered intravenously.

G. Methods of Diagnosing SC Viral Infection

In some embodiments, one or more MASC proteins as described herein can be used to detect SARS-CoV2 in a biological or non-biological sample. For example, MASC proteins reagents can be used in assays to detect the presence or absence of, or protein expression levels, for SARS-CoV2 using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the protein concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the MASC proteins to SARS-CoV2 can be detected directly or indirectly. Direct labels include fluorescent or luminescent tags, metals, dyes, radionuclides, and the like, attached to the antibody. A MASC protein labeled with iodine-125 (1251) can be used. A chemiluminescence assay using a chemiluminescent antibody specific for the nucleic acid is suitable for sensitive, non-radioactive detection of protein levels. A MASC proteins labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), (β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of 125I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The MASC proteins can be immobilized onto a variety of solid supports, such as magnetic or chromatographic matrix particles, the surface of an assay plate (e.g., microtiter wells), pieces of a solid substrate material or membrane (e.g., plastic, nylon, paper), in the physical form of sticks, sponges, papers, wells, and the like. An assay strip can be prepared by coating the antibody or a plurality of antibodies in an array on a solid support. This strip can then be dipped into the test sample and processed quickly through washes and detection steps to generate a measurable signal, such as a colored spot.

H. Methods of Screening for Antigen Binding Domains

Also provided herein are methods to screen for other ABDs that compete for binding with the present MASC proteins. The term “compete”, as used herein with regard to an ABD, means that a first ABD, or an antigen-binding portion thereof, competes for binding with a second ABD, or an antigen-binding portion thereof, where binding of the first ABD with its cognate epitope is detectably decreased in the presence of the second ABD compared to the binding of the first ABD in the absence of the ABD antibody. The alternative, where the binding of the second ABD to its epitope is also detectably decreased in the presence of the first ABD, can, but need not be the case. That is, a first ABD can inhibit the binding of a second ABD to its epitope without that second ABD inhibiting the binding of the first ABD to its respective epitope. However, where each ABD detectably inhibits the binding of the other ABD with its cognate epitope or ligand, whether to the same, greater, or lesser extent, the ABDs are said to “cross-compete” with each other for binding of their respective epitope(s). Both competing and cross-competing ABD are encompassed by the present invention. Regardless of the mechanism by which such competition or cross-competition occurs (e.g., steric hindrance, conformational change, or binding to a common epitope, or portion thereof, and the like), the skilled artisan would appreciate, based upon the teachings provided herein, that such competing and/or cross-competing ABDs are encompassed and can be useful for the methods disclosed herein.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, Antibodies, A Laboratory Manual, Cold Spring Harbor Press (1988)); solid phase direct label RIA using I-125 label (see Morel et al., Molec. Immunol. 25(1):7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified antigen bound to a solid surface or cells bearing either of these, an unlabelled test immunoglobulin and a labeled reference immunoglobulin. Competitive inhibition is measured by determining the amount of label bound to the solid surface or cells in the presence of the test immunoglobulin. Usually the test immunoglobulin is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit specific binding of a reference antibody to a common antigen by at least 50 or 75%.

Competitive binding assays can be used to identify antibodies that compete with an antibody described herein for specific binding to the SARS-CoV2 virus. Any of a number of competitive binding assays known in the art can be used to measure competition between two antibodies to the same antigen. Briefly, the ability of different antibodies to inhibit the binding of another antibody is tested. For example, antibodies can be differentiated by the epitope to which they bind using a sandwich ELISA assay. This is carried out by using a capture antibody to coat the surface of a well. A subsaturating concentration of tagged-antigen is then added to the capture surface. This protein will be bound to the antibody through a specific antibody:epitope interaction. After washing a second antibody, which has been covalently linked to a detectable moiety (e.g., HRP, with the labeled antibody being defined as the detection antibody) is added to the ELISA. If this antibody recognizes the same epitope as the capture antibody it will be unable to bind to the target protein as that particular epitope will no longer be available for binding. If however this second antibody recognizes a different epitope on the target protein it will be able to bind and this binding can be detected by quantifying the level of activity (and hence antibody bound) using a relevant substrate. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine epitope specificity.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

### IV. EXAMPLES

### 1.1 Example 1

### Identification and Characterization of Parental MASC Proteins

(a) Binding of Yeast-Surface Displayed Nanobodies to SARS-CoV2 Spike Ectodomain

Yeast displaying a single nanobody clone on the surface were assessed for binding to purified fluorescently labeled SARS-CoV2 Spike ectodomain by flow cytometry. Approximately 1×10⁶ yeast were incubated with SARS-CoV2 Spike ectodomain (Spike ECD) or the receptor binding domain (RBD) labeled with Alexa647 for 30 minutes at 25 C. After extensive washing of yeast by repeated centrifugation and resuspension, the amount of SARS-CoV2 Spike ectodomain bound to yeast cells was measured by flow cytometry. Nanobody clones that bind to SARS-CoV2 Spike ectodomain showed strong fluorescence signal in the Alexa647 channel. Binding to SARS-CoV2 Spike ectodomain was decreased in the presence of 1.4 micromolar purified ACE2-Fc, indicative of an epitope that is competitive with human ACE2.

TABLE 1

| | | Spike | Spike ECD + |
|---|---|---|---|
| | RBD- | ECD | ACE2-Fc |
| | (10 nM) | (1 nM) | (1.4 μM) |
| pNbCOV001B | +++ | +++ | − |
| pNbCOV005A | +++ | +++ | − |
| pNbCOV006A | +++ | +++ | − |
| pNbCOV008C | ++ | + | − |
| pNbCOV010B | ++ | +++ | + |
| pNbCOV011A | +++ | +++ | − |
| pNbCOV012C | +++ | +++ | − |
| pNbCOV015A | ++ | +++ | − |
| pNbCOV016A | +++ | +++ | − |
| pNbCOV019A | + | +++ | − |
| pNbCOV021A | ++ | +++ | − |
| pNbCOV022A | + | +++ | − |
| pNbCOV024B | + | +++ | − |
| pNbCOV002C | − | +++ | − |
| pNbCOV003A | − | +++ | + |
| pNbCOV004B | − | +++ | − |
| pNbCOV013A | − | +++ | + |
| pNbCOV017A | − | +++ | − |
| pNbCOV018A | − | +++ | − |

+++: Strong signal
++: Medium signal
+: Low signal
−: No Signal

(b) Surface Plasmon Resonance of Nanobodies to Spike Ectodomain (ECD).

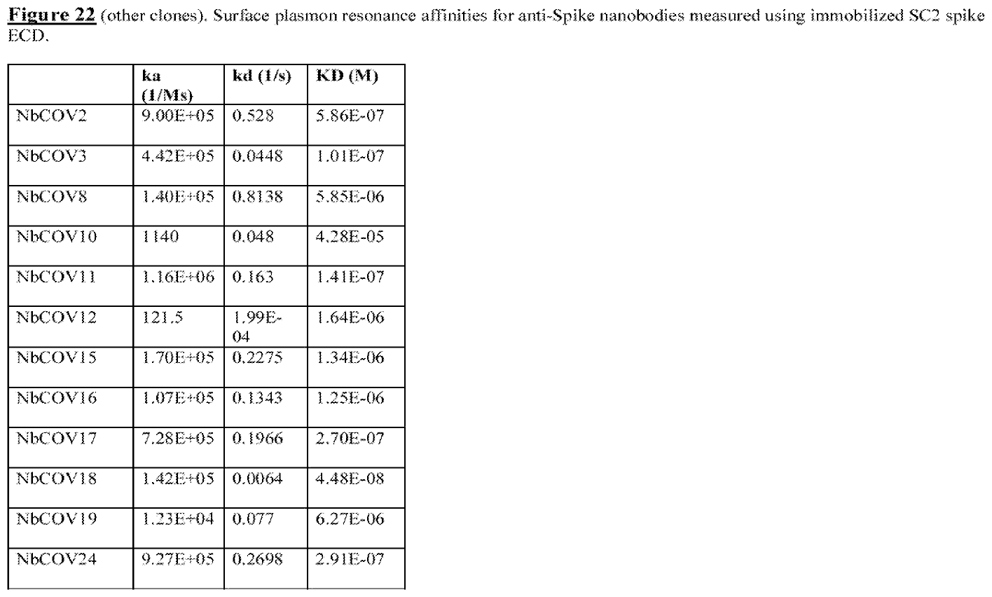

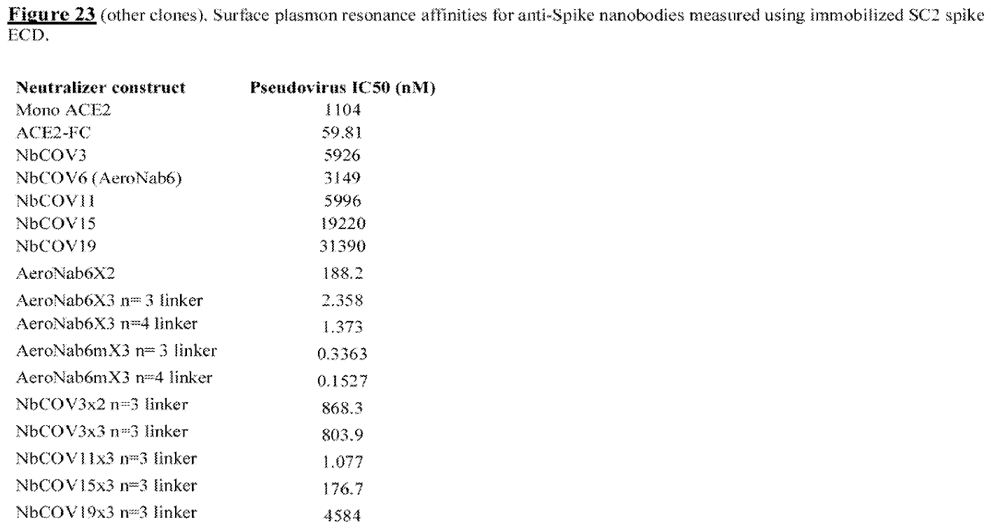

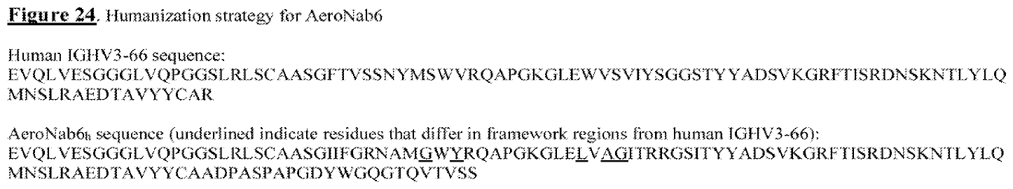

Stabilized SARS-CoV2 ectodomain bearing a C-terminal 8× histidine tag and a Twin-strep-tag was expressed in Expi293 cells and purified using metal-affinity chromatography and size exclusion chromatography. This antigen was captured on a Cytiva surface plasmon resonance chip via Streptactin XT to analyze the kinetic properties of nanobodies raised against the SARS-CoV2 Spike ectodomain. The results are shown in FIG. 22.

### 1.2 Example 2

### Affinity Maturation

One of the clones described in Example 1 was affinity-matured. The original clone was pNbCOV006A, with the sequence as follows (CDRs underlined):

QVQLVESGGGLVQAGGSLRLSCAASGIIFGRNAMGWYRQAPGKERELVA
GITRRGSITYYADSVKGRFTISRDNAKNTVYLQMNSLKPEDTAVYYCAA
DPASPAPGDYWGQGTQVTVSS

(a) Affinity Maturation Process:

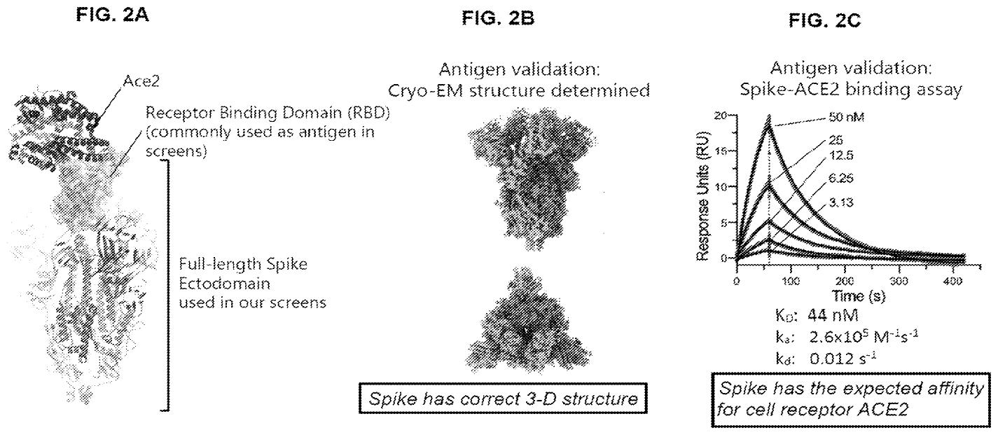

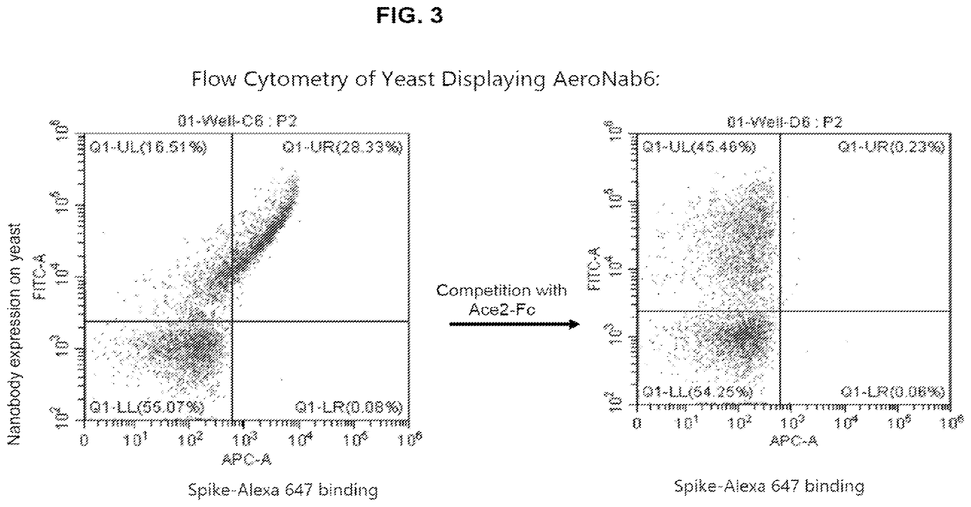

A saturation mutagenesis library of the original clone was generated by degenerate oligonucleotides encoding all 20 amino acids at each position within CDR1, CDR2, and CDR3. This library of variants was displayed on the surface of yeast. High affinity clones were progressively selected with stringent criteria, i.e. decreasing concentrations of the SARS-Cov2 Spike protein receptor binding domain (RBD). After two rounds of selection, a pool of yeast displaying nanobody variants showed higher affinity binding to the Spike RBD compared to the parent nanobody as outlined in the FIG. 2.

(b) Affinity Maturation Library Outcome:

Eight individual clones were sequenced from this pool, demonstrating that mutations at the following positions were responsible for improved affinity. Additionally, combinations of the substitutions below may yield improvements in affinity to the parent clone.

CDR1

Original: GIIFGRNA

Substitutions to position 3: Y/W/F/V/L

CDR2

Original: TRRGSITY

Substitutions to position 4: H/Y/G/Q

CDR3

Original: AADPASPAPGDY

Substitutions to position 6: V/L/I/T

Substitutions to position 9: F/W/Y/L/V

Based on sequence convergence, we specifically tested the activity of one clone (mNbCOV6). Underlines represent substitutions compared to the parent:

QVQLVESGGGLVQAGGSLRLSCAASGYIFGRNAMGWYRQAPGKERELVA
GITRRGSITYYADSVKGRFTISRDNAKNTVYLQMNSLKPEDTAVYYCAA
DPASPAYGDYWGQGTQVTVSS

CDR1
GYIFGRNA
CDR2
TRRGSITY
CDR3
AADPASPAYGDY

mNbCOV6 is significantly more potent than the parent clone NbCOV6. HEK293 cells expressing the angiotensin converting enzyme 2 (ACE2) receptor were incubated with 1 nM purified, stabilized SARS-CoV2 Spike ectodomain fluorescently conjugated with an Alexa 647 dye in the presence of increasing concentrations of either the parent nanobody (NbCOV6) or the affinity matured nanobody (mNbCOV6). NbCOV6 inhibited Spike ectodomain binding with an EC50 of 359 nM while the affinity matured nanobody (mNbCOV6) has an EC50 of 0.056 nM. The same assay was repeated with fluorescently labeled SARS-CoV2 Spike receptor binding domain (RBD). The parent NbCOV6 inhibited RBD binding with an EC50 of 190 nM while the affinity mNbCOV6 inhibited with an EC50 of 1.5 nM.

TABLE 3

| | RBD inhibition | | Spike trimer inhibition | |
|---|---|---|---|---|
| | EC50 | | EC50 | |
| Parent clone | 190 | nM | 359 | nM |
| (NbCOV6) | | | | |
| Affinity matured | 1.5 | nM | 0.056 | nM |
| (mNbCOV6) | | | | |

### 1.3 Example 3

### Pseudovirus Neutralization Assay

ZsGreen SARS-CoV-2-pseudotyped lentivirus was generated according to a published protocol (https://www.mdpi.com/1999-4915/12/5/513). The day before transduction, 50,000 HEK293T-ACE2 cells were plated in each well of a 24-well plate. 10-fold serial dilutions of nanobody were generated in complete medium (DMEM+10% FBS+PSG) and pseudotyped virus was added to a final volume of 200 ul. The media over the cells was replaced with nanobody/pseudotyped virus mixture for four hours, then removed. Cells were washed with complete medium and then incubated in complete medium. 3 days post-transduction, cells were trypsinized and the proportion of ZsGreen+cells was measured on an Attune flow cytometer (Thermo Fisher).

### 1.4 Example 4

### An Ultra-Potent Synthetic Nanobody Neutralizes SARS-CoV-2 by Stabilizing Inactive Spike

The SARS-CoV-2 virus enters host cells via an interaction between its Spike protein and the host cell receptor angiotensin converting enzyme 2 (ACE2). By screening a yeast surface-displayed library of synthetic nanobody sequences, we developed nanobodies that disrupt the interaction between Spike and ACE2. Cryogenic electron microscopy (cryo-EM) revealed that one nanobody, Nb6, binds Spike in a fully inactive conformation with its receptor binding domains (RBDs) locked into their inaccessible down-state, incapable of binding ACE2. Affinity maturation and structure-guided design of multivalency yielded a trivalent nanobody, mNb6-tri, with femtomolar affinity for Spike and picomolar neutralization of SARS-CoV-2 infection. mNb6-tri retains function after aerosolization, lyophilization, and heat treatment, which enables aerosol-mediated delivery of this potent neutralizer directly to the airway epithelia.

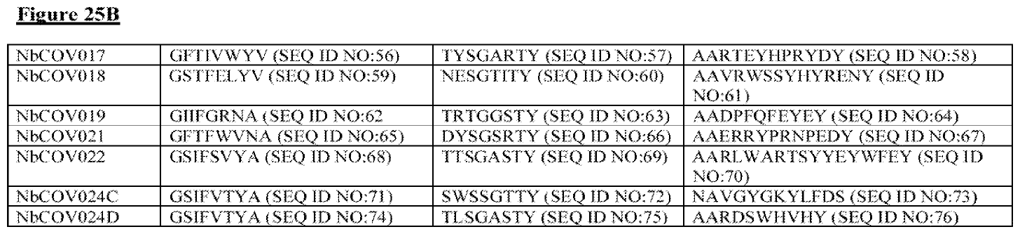

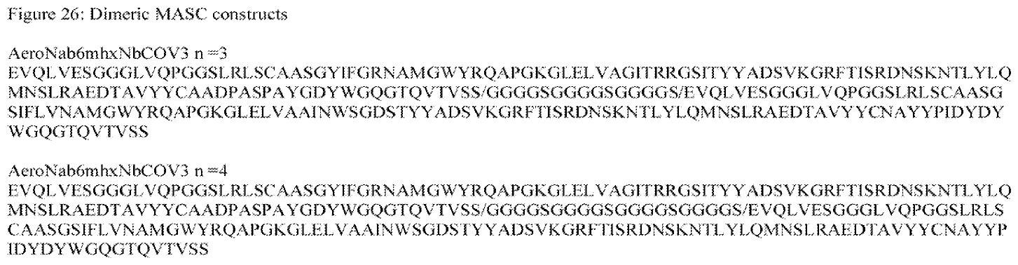

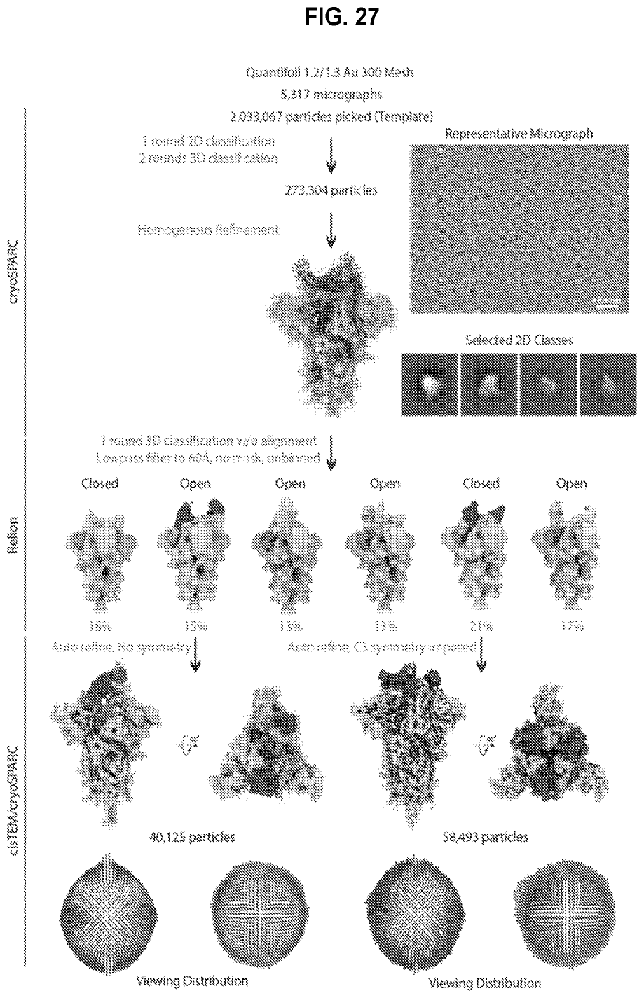

Single domain antibodies (nanobodies) were isolated that neutralize SARS-CoV-2 by screening a yeast surface-displayed library of >2×10⁹ synthetic nanobody sequences for binders to the Spike ectodomain (17). A mutant form of SARS-CoV-2 Spike (Spike^{S2P}) was used as the antigen (15). Spike^{S2P} lacks one of the two proteolytic cleavage sites between the S1 and S2 domains and introduces two mutations and a trimerization domain to stabilize the pre-fusion conformation. Spike^{S2P} was labeled with biotin or with fluorescent dyes and selected nanobody-displaying yeast over multiple rounds, first by magnetic bead binding and then by fluorescence-activated cell sorting (FIG. 27A).

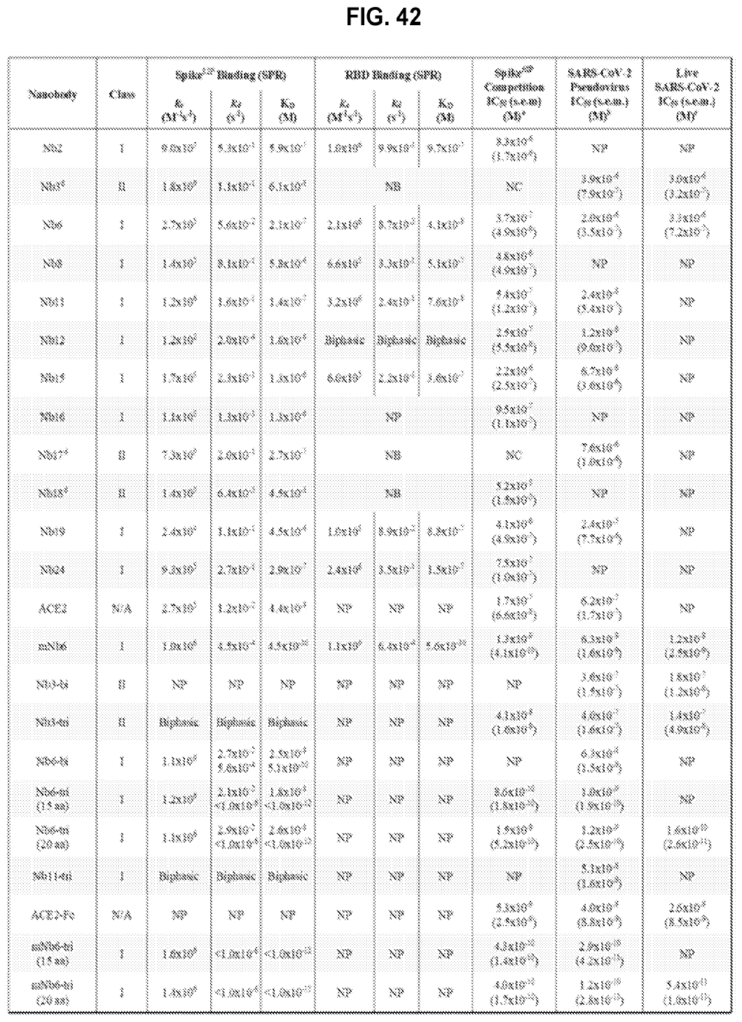

Three rounds of selection yielded 21 unique nanobodies that bound Spike^{S2P} and showed decreased binding in the presence of a dimeric construct of the ACE2 extracellular domain (ACE2-Fc). These nanobodies fall into two classes. Class I binds the RBD and competes directly with ACE2-Fc (FIG. 27B). A prototypical example of this class is nanobody Nb6, which binds to Spike^{S2P} and to RBD alone with a KD of 210 nM and 41 nM, respectively (FIG. 27C; FIG. 42). Class II, exemplified by nanobody Nb3, binds to Spike^{S2P} (KD=61 nM), but displays no binding to RBD alone (FIG. 27C, FIG. 42). In the presence of excess ACE2-Fc, binding of Nb6 and other Class I nanobodies is blocked entirely, whereas binding of Nb3 and other Class II nanobodies is moderately decreased (FIG. 27B). These results suggest that Class I nanobodies target the RBD to block ACE2 binding, whereas Class II nanobodies target other epitopes. Indeed, surface plasmon resonance (SPR) experiments demonstrate that Class I and Class II nanobodies can bind Spike^{S2P} simultaneously (FIG. 27D).

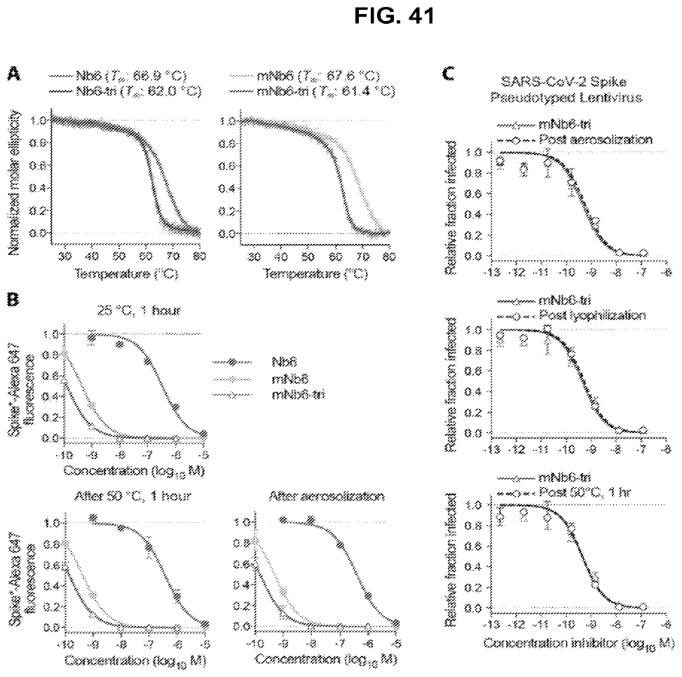

Class I nanobodies show a consistently faster association rate constant (ka) for nanobody binding to the isolated RBD than to Spike^{S2P} (FIG. 41), which suggests that RBD accessibility influences the KD. Next, the efficacy of Class I and Class II nanobodies was tested to inhibit binding of fluorescently labeled Spike^{S2P} to ACE2-expressing HEK293 cells (FIG. 27E; FIG. 42). Class I nanobodies Nb6 and Nb11 emerged as two of the most potent clones with IC50 values of 370 and 540 nM, respectively. Class II nanobodies showed little to no activity in this assay. Two Class I nanobodies were prioritized, Nb6 and Nb11, that combine potent Spike^{S2P} binding with relatively small differences in Ka between binding to Spike^{S2P} or RBD. For Class II nanobodies, we prioritized Nb3 because of its relative yield during purification (FIG. 42).

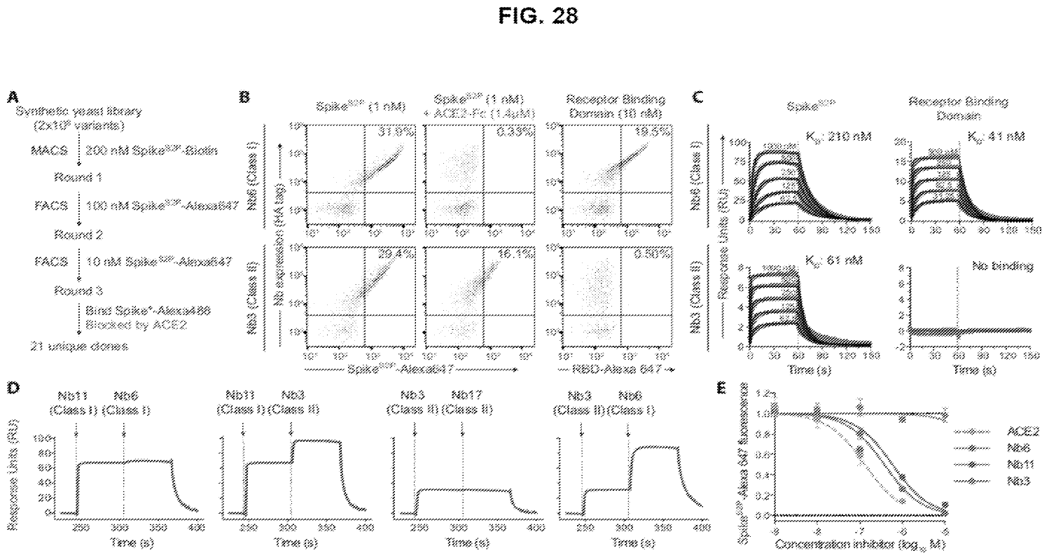

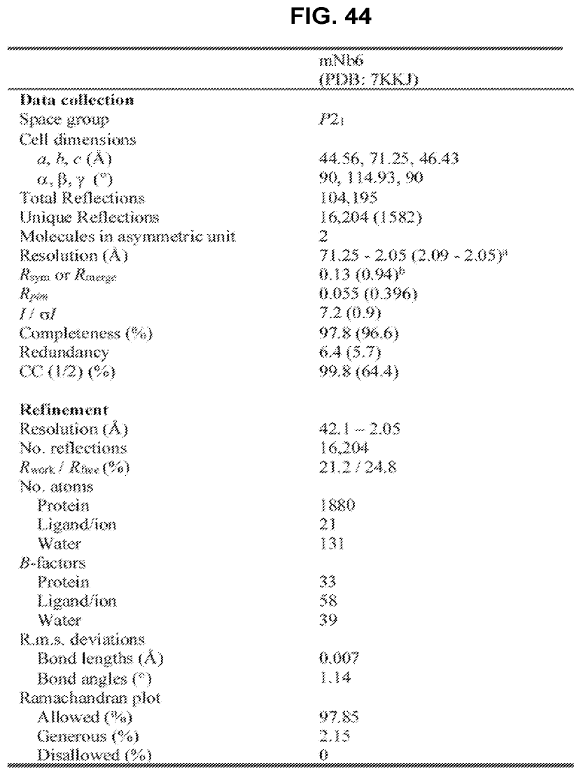

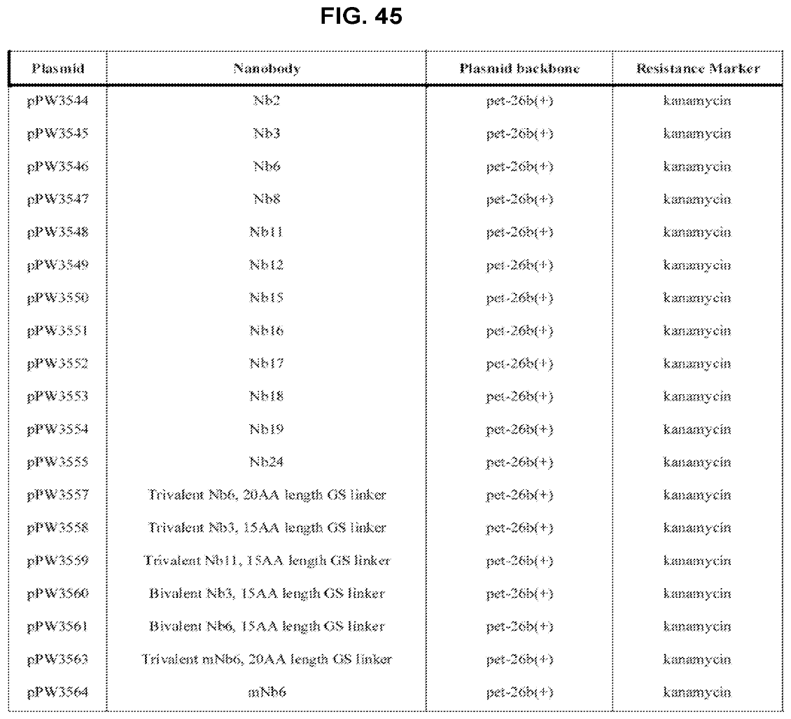

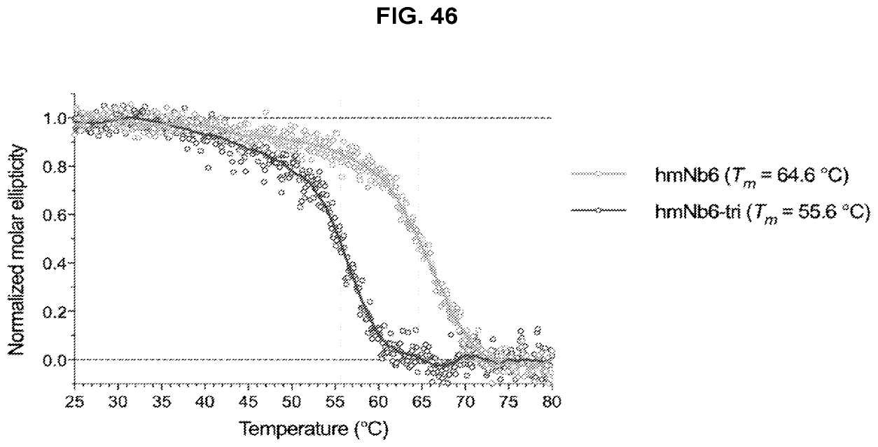

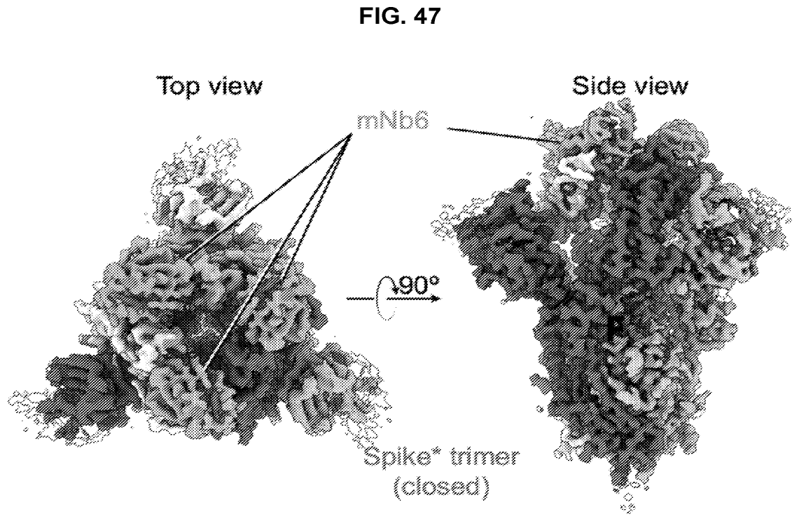

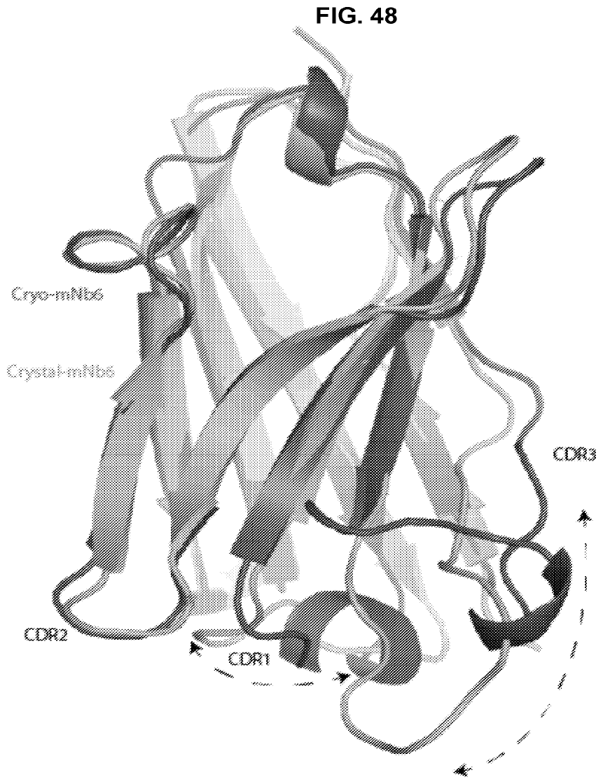

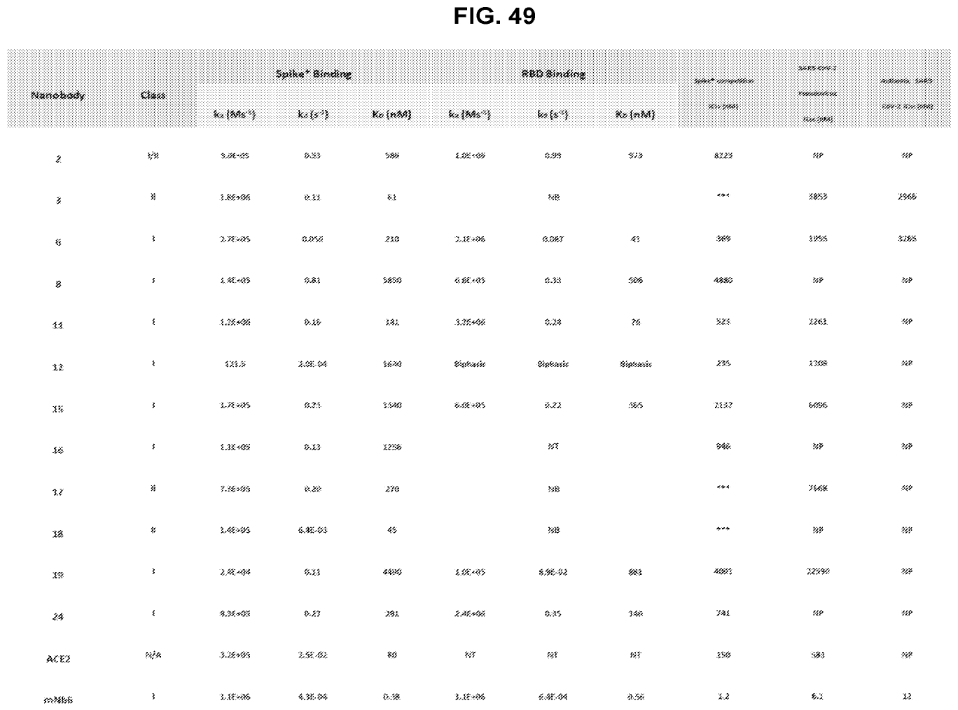

To define the binding sites of Nb6 and Nb11, the cryogenic electron microscopy (cryo-EM) structures bound to Spike^{S2P} were determined (FIG. 28A-B; FIG. 27-29; FIG. 44). Both nanobodies recognize RBD epitopes that overlap the ACE2 binding site (FIG. 28E). For Nb6 and Nb11, we resolved nanobody binding to both the open and closed conformations of Spike^{S2P}. We obtained a 3.0 Å map of Nb6 bound to closed Spike^{S2P}, which enabled modeling of the Nb6-Spike^{S2P} complex (FIG. 28A), including the complementarity determining regions (CDRs). We also obtained lower resolution maps for Nb6 bound to open Spike^{S2P} (3.8 Å), and Nb11 bound to open and closed Spike^{S2P} (4.2 Å, and 3.7 Å, respectively). For these lower resolution maps, we could define the nartobody's binding orientation but not accurately model the CDRs.

Nb6 bound to closed Spike^{S2P} straddles the interface between two adjacent RBDs. The majority of the contacting surfaces are contributed by CDR1 and CDR2 of Nb6 (FIG. 28C). CDR3 contacts the adjacent RBD positioned counterclockwise when viewed from the top (FIG. 28C). The binding of one Nb6 therefore stabilizes two adjacent RBDs in the down-state and likely pre-organizes the binding site for a second and third Nb6 molecule to stabilize the closed Spike conformation. By contrast, Nb11 bound to down-state RBDs only contacts a single RBD (FIG. 28D).

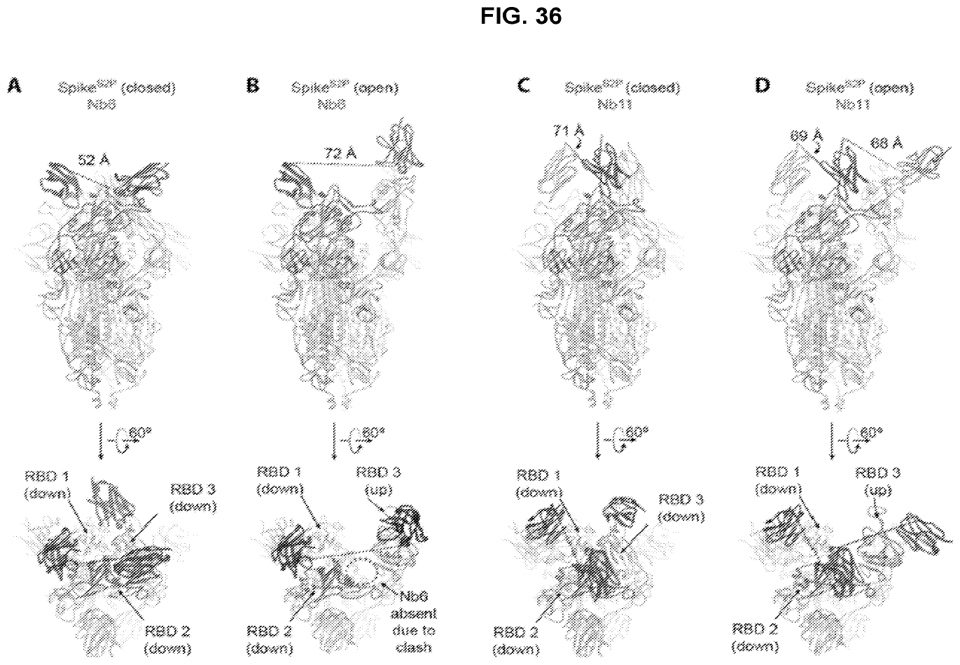

The structure of Nb6 bound to closed Spike^{S2P} enabled us to engineer bivalent and trivalent nanobodies predicted to lock all RBDs in the down-state. We inserted flexible Gly-Ser linkers of either 15 or 20 amino acids to span the 52 Å distance between adjacent Nb6 monomers bound to down-state RBDs in closed Spike^{S2P} (FIG. 36). These linkers are too short to span the 72 Å distance between Nb6 molecules bound to open Spike. Moreover, steric clashes would prevent binding of three RBDs in open Spike with a single up-state RBD even with longer linker length (FIG. 36). By contrast, the minimum distance between adjacent Nb11 monomers bound to either open or closed Spike^{S2P} is 68 Å. We predicted that multivalent binding by Nb6 constructs would display significantly slowed dissociation rates due to enhanced avidity.

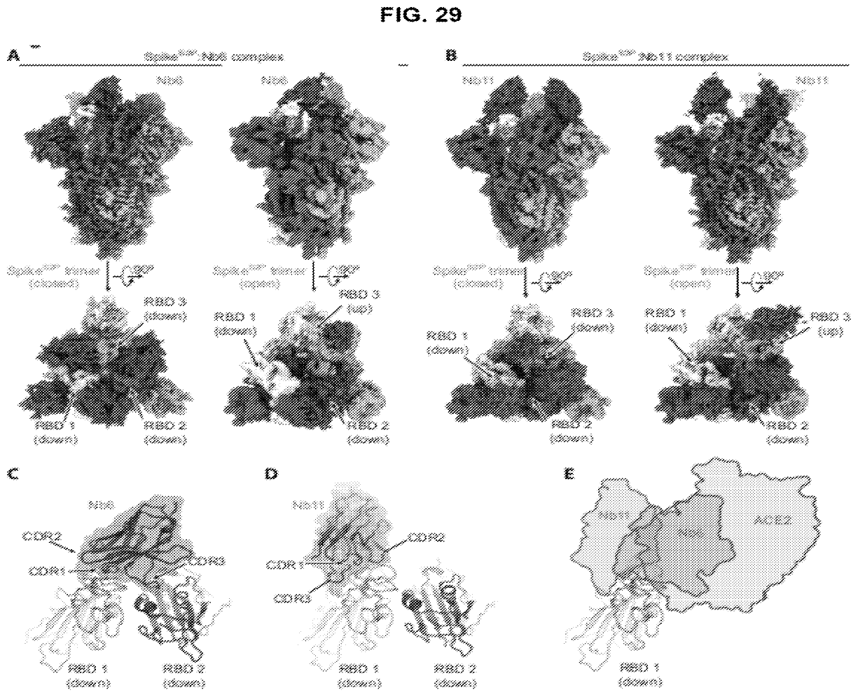

In SPR experiments, both bivalent Nb6 with a 15 amino acid linker (Nb6-bi) and trivalent Nb6 with two 20 amino acid linkers (Nb6-tri) dissociate from Spike^{S2P} in a biphasic manner. The dissociation phase can be fitted to two components: a fast phase with kinetic rate constants kd1 of 2.7×10-2s-1 for Nb6-bi and 2.9×10-2s-1 for Nb6-tri, which are close to that observed for monovalent Nb6 (kd=5.6×10-2s-1) and a slow phase that is dependent on avidity (kd2=3.1×10-4s-1 for Nb6-bi and kd2<1.0×10-6s-1 for Nb6-tri, respectively) (FIG. 29A). The relatively similar Kd for the fast phase suggests that a fraction of the observed binding for the multivalent constructs is nanobody binding to a single Spike^{S2P} RBD. By contrast, the slow dissociation phase of Nb6-bi and Nb6-tri indicates engagement of two or three RBDs. No dissociation was observed for the slow phase of Nb6-tri over 10 minutes, indicating an upper boundary for kd2 of 1×10-6s-1 and subpicomolar affinity. This measurement remains an upper boundary estimate because the measurement is limited by the intrinsic dissociation rate of Spike^{S2P} from the SPR chip imposed by the chemistry used to immobilize Spike^{S2P}. The true dissociation rate, therefore, may be significantly lower.

Biphasic dissociation could be explained by a slow interconversion between up- and down-state RBDs, with conversion to the more stable down-state required for multivalent binding: a single domain of Nb6-tri engaged with an up-state RBD would dissociate rapidly. The system would then re-equilibrate as the RBD flips into the down-state, eventually allowing Nb6-tri to trap all RBDs in closed Spike^{S2P}. To test this directly, the association time was varied for Nb6-tri binding to Spike^{S2P}. Indeed, we observed an exponential decrease in the percent fast-phase with a t^{1/2} of 65 s (FIG. 29B), which, we surmise, reflects the timescale of conversion between the RBD up- and down-states in Spike^{S2P}. Taken together, dimerization and trimerization of Nb6 afforded 750-fold and >200,000-fold gains in KD, respectively.

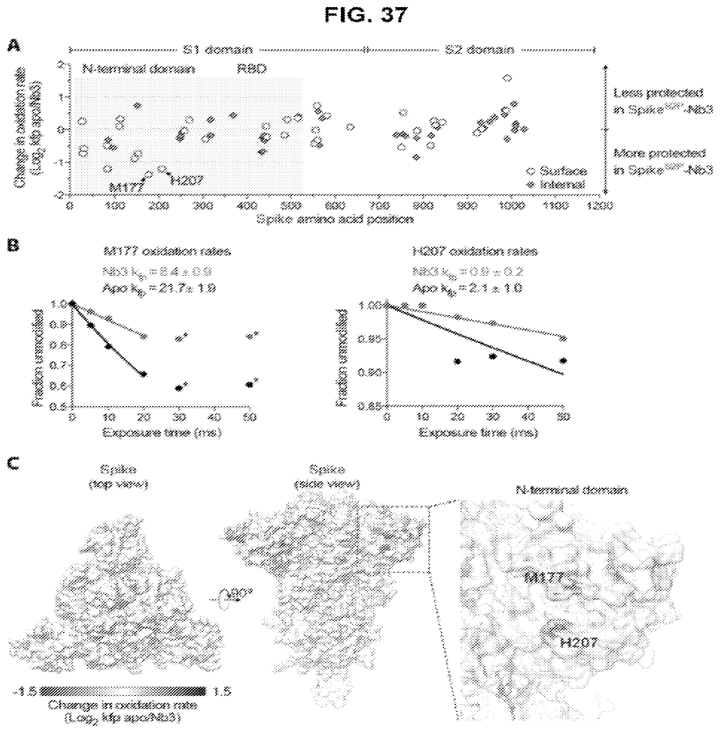

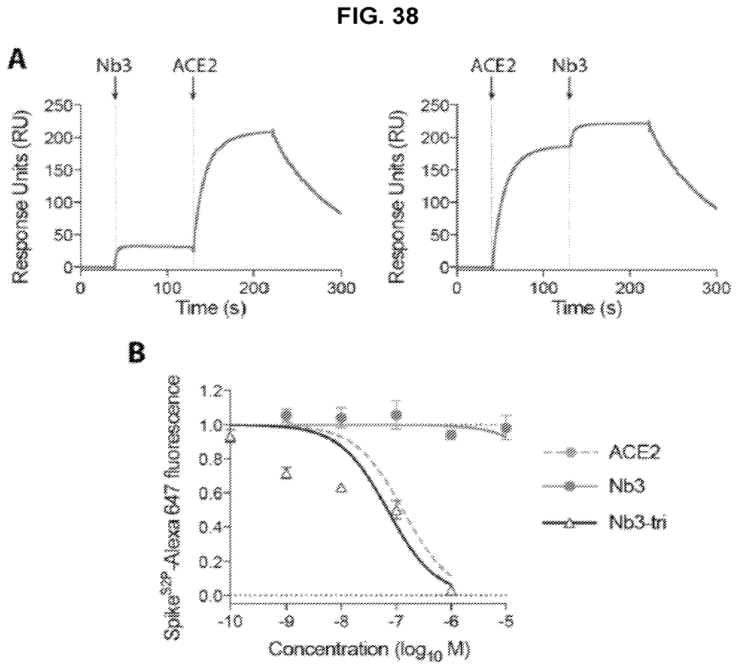

Unable to determine the binding site of Nb3 by cryo-EM, radiolytic hydroxyl radical footprinting was used. apo- or Nb3-bound Spike^{S2P} were exposed to synchrotron X-ray radiation to label solvent-exposed amino acids with hydroxyl radicals, which was subsequently quantified by mass spectrometry of protease digested Spike^{S2P}(18). Two neighboring surface residues on the S1 N-terminal domain of Spike (M177 and H207) were protected in the presence of Nb3 at a level consistent with prior observations of antibody-antigen interactions by hydroxyl radical footprinting (FIG. 37)(19). Previously discovered coronavirus neutralizing antibodies bind an epitope within the N-terminal domain of Spike with Fab fragments that are non-competitive with the host cell receptor (20, 21). Further SPR experiments demonstrated that Nb3 can bind Spike^{S2P} simultaneously with monovalent ACE2 (FIG. 38). It was hypothesized that multivalent display of Nb3 on the surface of yeast may account for the partial decrease in Spike^{S2P} binding observed in the presence of ACE2-Fc. Indeed, a trivalent construct of Nb3 with 15 amino acid linkers (Nb3-tri) inhibited Spike^{S2P} binding to ACE2 cells with an IC50 of 41 nM (FIG. 38). How Nb3-tri disrupts Spike-ACE2 interactions remains unclear.

Next, the neutralization activity of monovalent and trivalent versions of our top Class I (Nb6 and Nb11) and Class II (Nb3) nanobodies was tested against SARS-CoV-2 pseudotyped lentivirus using a previously described assay (22). Nb6 and Nb11 inhibited pseudovirus infection with IC50 values of 2.0 μM and 2.4 μM, respectively. Nb3 inhibited pseudovirus infection with an IC50 of 3.9 μM (FIG. 29C, FIG. 42). Nb6-tri shows a 2000-fold enhancement of inhibitory activity, with an IC50 of 1.2 nM, whereas trimerization of Nb11 and Nb3 resulted in more modest gains of 40- and 10-fold (51 nM and 400 nM), respectively (FIG. 29C). The neutralization activities were confirmed with a viral plaque assay using live SARS-CoV-2 virus infection of VeroE6 cells. Here, Nb6-tri proved exceptionally potent, neutralizing SARS-CoV-2 with an average IC50 of 160 pM (FIG. 29D). Nb3-tri neutralized SARS-CoV-2 with an average IC50 of 140 nM (FIG. 29D).

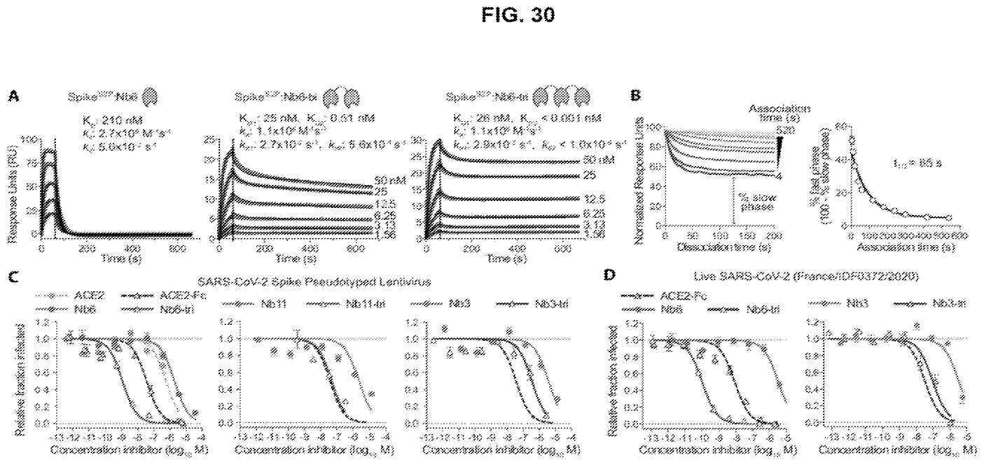

The potency of Nb6 was optimized by selecting a saturation mutagenesis library targeting all three CDRs. Two rounds of selection identified high-affinity clones with two penetrant mutations: I27Y in CDR1 and P105Y in CDR3. We incorporated these mutations into Nb6 to generate matured Nb6 (mNb6), which binds with 500-fold increased affinity to Spike^{S2P} (FIG. 30A). mNb6 inhibits both pseudovirus and live SARS-CoV-2 infection with low nanomolar potency, a ˜200-fold improvement compared to Nb6 (FIG. 30B; FIG. 42).

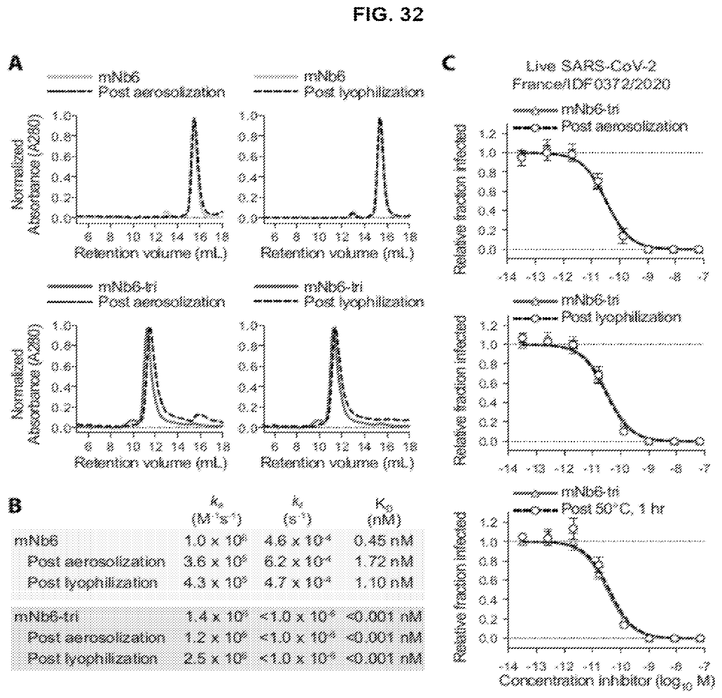

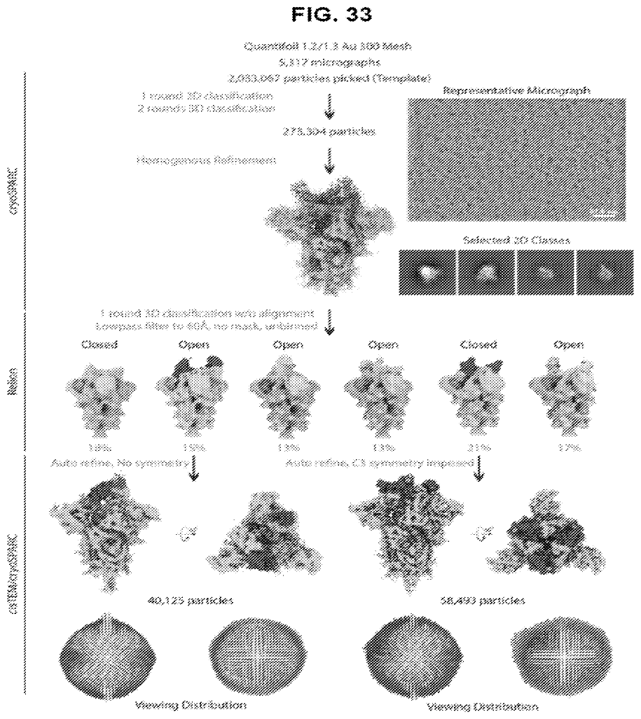

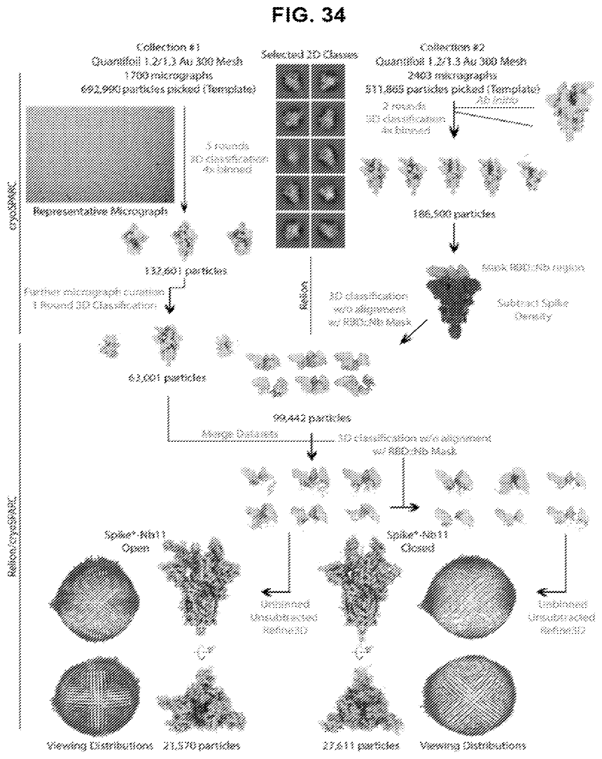

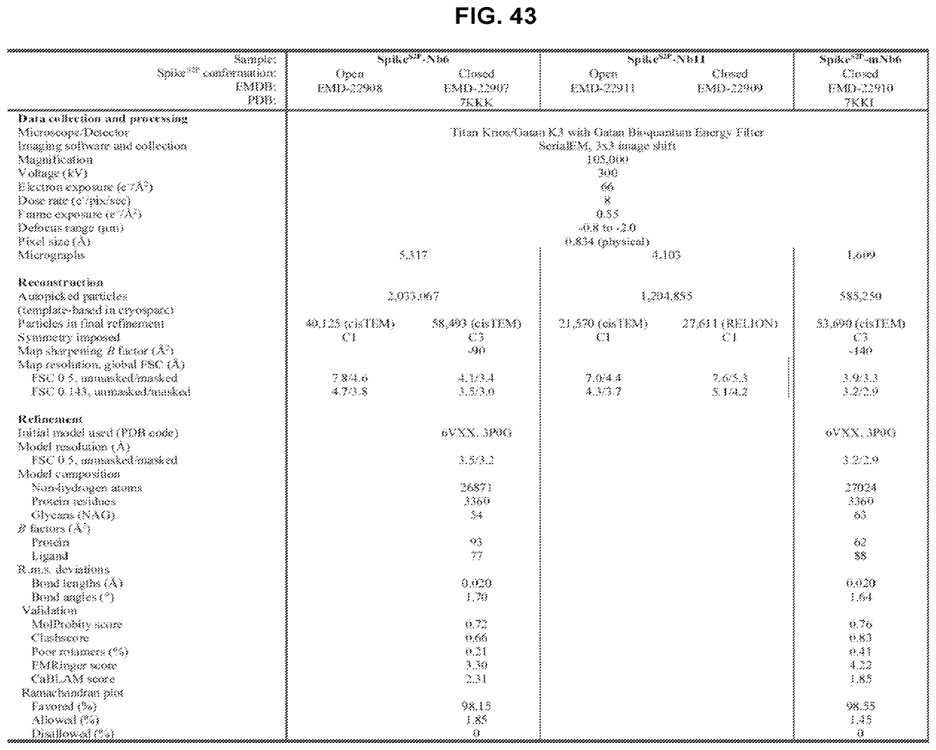

A 2.9 Å cryo-EM structure shows that mNb6 binds to closed Spike^{S2P} (FIG. 30C; FIG. 32). mNb6 induces a slight rearrangement of the down-state RBDs as compared to Spike^{S2P} bound to Nb6, inducing a 9° rotation of the RBD away from the central three-fold symmetry axis. This deviation likely arises from a different interaction between CDR3 and Spike^{S2P}, which nudges the RBDs into a new resting position (FIG. 30D). While the I27Y substitution optimizes local contacts between CDR1 in its original binding site on the RBD, the P105Y substitution leads to a marked rearrangement of CDR3 in mNb6 (FIG. 30E-F). This conformational change yields a different set of contacts between mNb6 CDR3 and the adjacent RBD. An X-ray crystal structure of mNb6 alone revealed dramatic conformational differences in CDR1 and CDR3 between free and Spike^{S2P}-bound mNb6 (FIG. 30G; FIG. 43). Although differences in loop conformation in the crystal structure may arise from crystal lattice contacts, they are suggestive of conformational heterogeneity for unbound mNb6 and induced-fit rearrangements upon binding to Spike^{S2P}.

The binding orientation of mNb6 is similar to that of Nb6, suggesting that multivalent design would likewise enhance binding affinity. Unlike Nb6-tri, trivalent mNb6 with a 20 amino acid linker (mNb6-tri) bound to Spike^{S2P} with no observable fast-phase dissociation and no measurable dissociation over ten minutes, yielding an upper bound for the dissociation rate constant kd of 1.0×10-6s-1 (t½>8 days) and a KD of <1 pM (FIG. 30A). mNb6-tri displays further gains in potency in both pseudovirus and live SARS-CoV-2 infection assays with IC50 values of 120 pM (5.0 ng/mL) and 54 pM (2.3 ng/mL), respectively (FIG. 30B, FIG. 41). Given the sub-picomolar affinity observed by SPR, it is likely that these viral neutralization potencies reflect the lower limit of the assays. mNb6-tri is therefore an exceptionally potent SARS-CoV-2 neutralizing molecule.

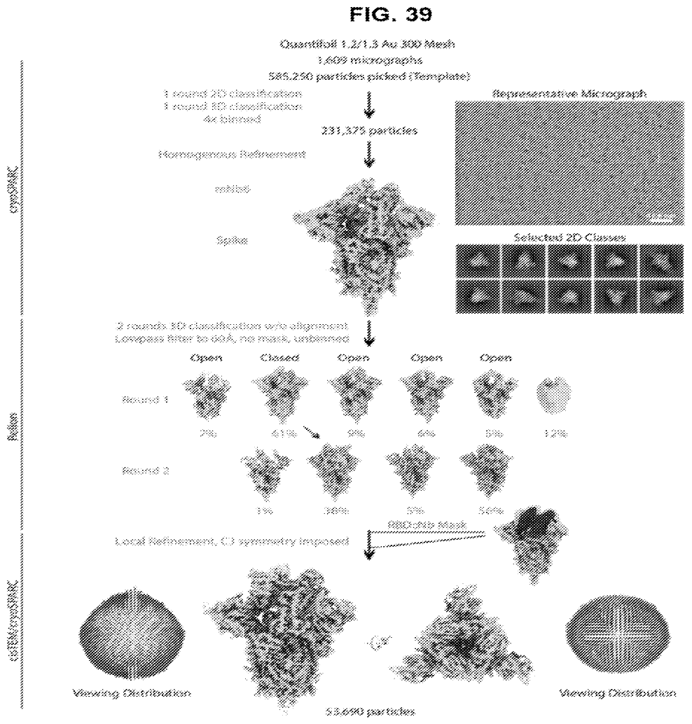

Next, viral neutralization by the Class I nanobody mNb6 was tested to see if it was potentially synergistic with the Class II nanobody Nb3-tri. In pseudovirus neutralization assays, an additive effect was observed when combining Nb3-tri with mNb6 (FIG. 39). However, the potency for mNb6 viral neutralization was unchanged with increasing concentrations of Nb3-tri, suggesting minimal synergy between these two nanobodies.

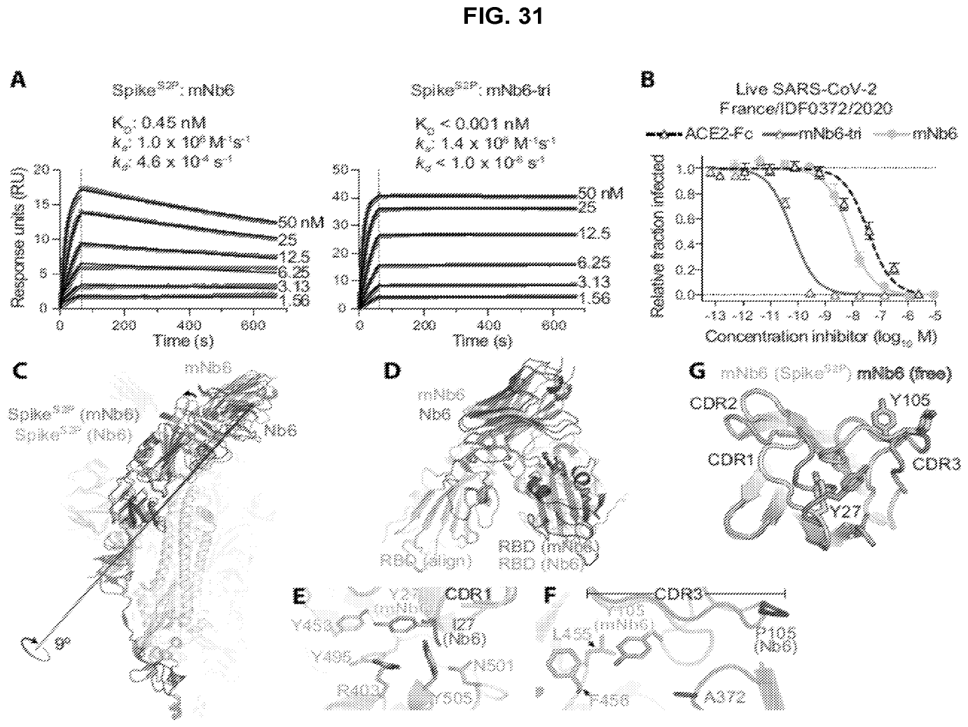

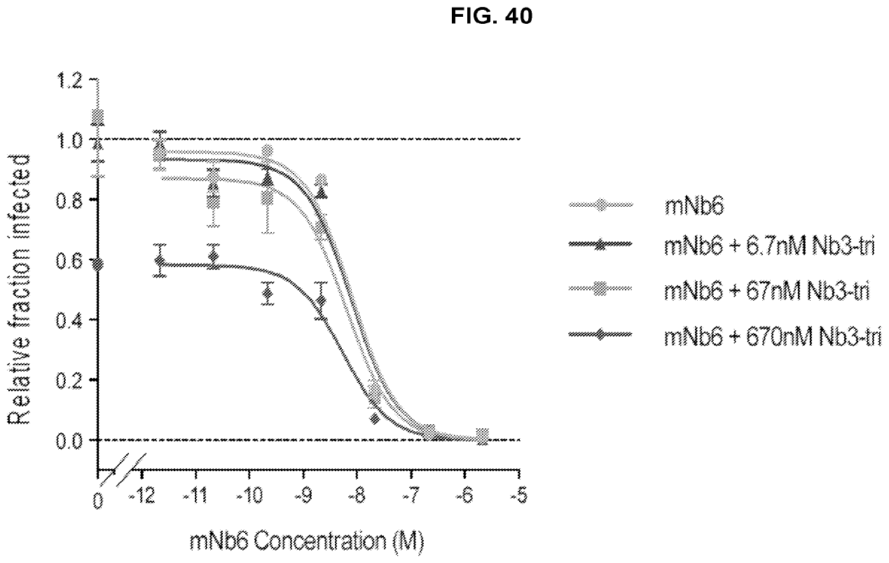

Next, Nb6 and its derivatives were tested for stability. Circular dichroism revealed melting temperatures of 66.9, 62.0, 67.6, and 61.4° C. for Nb6, Nb6-tri, mNb6 and mNb6-tri, respectively (FIG. 40). Moreover, mNb6 and mNb6-tri were stable to lyophilization and to aerosolization, showing no aggregation by size exclusion chromatography and preserved high affinity binding to Spike^{S2P} (FIG. 31A-B and FIG. 40). Finally, mNb6-tri retains potent inhibition of pseudovirus and live SARS-CoV-2 infection after aerosolization, lyophilization, or heat treatment for 1 hour at 50° C. (FIG. 31C and FIG. 40).

Strategies to prevent SARS-CoV-2 entry into the host cell aim to block the ACE2-RBD interaction (20, 23-30). Although high-affinity monoclonal antibodies are leading the way as potential therapeutics, they are expensive to produce by mammalian cell expression and need to be intravenously administered by healthcare professionals. Large doses are needed for prophylactic use, as only a small fraction of systemic antibodies cross the epithelial cell layers lining the airways (32). By contrast, nanobodies can be inexpensively produced in bacteria or yeast. The inherent stability of nanobodies enables aerosolized delivery directly to the nasal and lung epithelia (33). Indeed, aerosol delivery of a trimeric nanobody targeting respiratory syncytial virus (ALX-0171) was recently demonstrated to be effective in substantially decreasing measurable viral load in hospitalized infants (34). Finally, potential immunogenicity of camelid-derived nanobodies can be mitigated by established humanization strategies (35).

Nanobody multimerization has been shown to improve target affinity by avidity (33, 36). In the case of Nb6 and mNb6, structure-guided design of a multimeric construct that simultaneously engages all three RBDs yielded profound gains in potency. Furthermore, because RBDs must be in the up-state to engage with ACE2, conformational control of RBD accessibility serves as an added neutralization mechanism (30). Indeed, when mNb6-tri engages with Spike, it prevents ACE2 binding by both directly occluding the binding site and by locking the RBDs into an inactive conformation. Discovery of Class II neutralizing nanobodies demonstrates potentially novel mechanisms of disrupting Spike function. Pairing of Class I and Class II nanobodies in a prophylactic or therapeutic cocktail could provide both potent neutralization and prevention of escape variants. The combined stability, potency, and diverse epitope engagement of our anti-Spike nanobodies therefore provide a unique potential prophylactic and therapeutic strategy to limit the continued toll of the COVID-19 pandemic.

Materials and Methods:

1. Expression and Purification of SARS-CoV-2 Spike, RBD, and ACE2

A previously described construct was used to express and purify the pre-fusion SARS-CoV-2 Spike ectodomain (Spike^{S2P})(15). ExpiCHO or Expi293T cells (ThermoFisher) were transfected with the Spike^{S2P} construct per the manufacturer's instructions for the MaxTiter protocol and harvested between 3-9 days after transfection. Clarified cell culture supernatant was loaded onto Ni-Excel beads (Cytiva) followed by extensive washes in 20 mM HEPES pH 8.0, 200 mM sodium chloride, and 10 mM imidazole and elution in the same buffer supplemented with 500 mM imidazole. Spike^{S2P} was concentrated using a 100 kDa MWCO spin concentrator (Millipore) and further purified by size exclusion chromatography over a Superose 6 Increase 10/300 column (GE Healthcare) in 20 mM HEPES pH 8.0 and 200 mM sodium chloride. All purification steps were performed at room temperature. The resulting fractions for trimeric Spike^{S2P} were pooled and either used directly for cryo-EM studies or concentrated and flash frozen in liquid nitrogen with 15% glycerol for other biochemical studies.

We used a previously described construct to express and purify the SARS-CoV-2 Receptor binding domain (RBD)(37). Expi293T cells (ThermoFisher) were transfected with the RBD construct per the manufacturer's instructions and harvested between 3-6 days after transfection. Clarified cell culture supernatant was loaded onto Ni-Excel beads (Cytiva) or a His-Trap Excel column (GE Healthcare) followed by washes in 20 mM HEPES pH 8.0, 200 mM sodium chloride, and 10 mM imidazole and elution using the same buffer supplemented with 500 mM imidazole. RBD was concentrated using a 30 kDa MWCO spin concentrator (Millipore) and further purified by size exclusion chromatography over a Superdex 200 Increase 10/300 GL column (GE Healthcare) in 20 mM HEPES pH 8.0 and 200 mM sodium chloride. The resulting fractions were pooled, concentrated, and flash frozen in liquid nitrogen with 10% glycerol.

For biochemical and yeast display experiments, Spike^{S2P} and RBD were labeled with freshly prepared stocks of Alexa 647-NHS, Alexa 488-NHS, or Biotin-NHS (ThermoFisher) with a 5-fold stoichiometry for 1 hour at room temperature followed by quenching of NHS with 10 mM Tris pH 8.0 for 60 minutes. Labeled proteins were further purified by size exclusion chromatography, concentrated using a spin concentrator (Millipore), and flash frozen in liquid nitrogen with 10-15% glycerol.

We used an ACE2-ECD (18-614) Fc fusion expression plasmid to express and purify Fc tagged ACE2-ECD(38). Expi293T cells (ThermoFisher) were transfected with the ACE2-Fc construct per the manufacturer's instructions and harvested between 5-7 days after transfection. Clarified cell culture supernatant was loaded onto a MabSelect Pure 1 mL Column (GE Healthcare). Column was washed with Buffer A (20 mM HEPES pH 7.5, 150 mM NaCl) and protein was eluted with Buffer B (100 mM Sodium Citrate pH 3.0, 150 mM NaCl) into a deep well block containing 1 M HEPES pH 7.5 to neutralize the acidic elution. ACE2-Fc was concentrated using a 30 kDa MWCO spin concentrator (Millipore) and further purified by size exclusion chromatography over a Superdex 200 Increase 10/300 GL column (GE Healthcare) in SEC Buffer (20 mM HEPES pH 7.5, 150 mM NaCl, 5% v/v Glycerol). The resulting fractions were pooled, concentrated, and flash frozen in liquid nitrogen. To obtain monomeric ACE2, 1:50 (w/w) His-tagged TEV protease was added to ACE2-Fc and incubated at 4° C. overnight. This mixture was then purified by size exclusion chromatography in SEC Buffer. Monomeric ACE2 fractions were pooled and washed with His-resin (1 mL of 50% slurry) to remove excess TEV. The resulting supernatant was pooled, concentrated, and flash frozen in liquid nitrogen.

2. Identification of Anti SARS-CoV2 Spike Nanobodies

To identify nanobodies against the SARS-CoV-2 Spike ECD, we used a yeast surface displayed library of synthetic nanobody sequences that recapitulate amino acid position specific-variation in natural llama immunological repertoires. This library encodes a diversity of >2×10⁹ variants, and uses a synthetic stalk sequence for nanobody display, as described previously in a modified vector encoding nourseothricin (NTC) resistance(17). For the first round of selection, 2×10¹⁰ yeast induced in YPG (Yeast Extract-Peptone-Galactose) supplemented with NTC were washed repeatedly in selection buffer (20 mM HEPES, pH 7.5, 150 mM sodium chloride, 0.1% (w/v) low biotin bovine serum albumin, BSA) and finally resuspended in 10 mL of selection buffer containing 200 nM biotinylated-Spike^{S2P}. Yeast were incubated for 30 minutes at 25° C., then washed repeatedly in cold selection buffer, and finally resuspended in 10 mL of cold selection buffer containing 200 μL of Miltenyi anti-Streptavidin microbeads. After 30 minutes of incubation at 4° C., yeast were again washed with cold selection buffer. Spike^{S2P} binding yeast were captured on a Miltenyi MACS LS column and recovered in YPD (Yeast Extract-Peptone-Dextrose) medium supplemented with NTC.

For round 2, 4×10⁸ induced yeast from Round 1 were incubated with 100 nM Spike^{S2P} labeled with Alexa647 in 1 mL of selection buffer for 1 hr at 25° C. After extensive washes with cold selection buffer, Spike^{S2P} binding yeast were isolated by fluorescence activated cell sorting (FACS) on a Sony SH800 instrument. A similar approach was used for round 3, with substitution of 10 nM Spike^{S2P} labeled with Alexa647. Post round 3 yeast were plated on YPD+NTC solid media and 768 individual colonies were induced with YPG+NTC media in 2 mL deep well plates. Each individual clone was tested for binding to 4 nM Spike^{S2P}-Alexa488 by flow cytometry on a Beckman Coulter Cytoflex. To identify nanobodies that disrupt Spike-ACE2 interactions, Spike^{S2P} binding was repeated in the presence of 0.5-1 μM ACE2-Fc. Out of 768 clones, we identified 21 that strongly bind Spike^{S2P} and are competitive with ACE2 (FIG. 44).

3. Expression and Purification of Nanobodies

Nanobody sequences were cloned into the pET26-b(+) expression vector using In-Fusion HD cloning (Takara Bio), transformed into BL21(DE3) E. coli (New England BioLabs), grown in Terrific Broth at 37° C. until OD 0.7-0.8, followed by gene induction using 1 mM IPTG for 18-22 hours at 25° C. E. coli were harvested and resuspended in SET Buffer (200 mM Tris, pH 8.0, 500 mM sucrose, 0.5 mM EDTA, 1× cOmplete protease inhibitor (Roche)) for 30 minutes at 25° C. before a 45 minute osmotic shock with a two-fold volume addition of water. NaCl, MgCl2, and imidazole were added to the lysate to 150 mM, 2 mM, and 40 mM respectively before centrifugation at 17-20,000×g for 15 minutes to separate cell debris from the periplasmic fraction. For every liter of bacterial culture, the periplasmic fraction was then incubated with 4 mL of 50% HisPur Ni-NTA resin (Thermo Scientific) which had been equilibrated in Nickel Wash Buffer (20 mM HEPES, pH 7.5, 150 mM NaCl, 40 mM imidazole). This mixture was incubated for 1 hr with rotation at RT before centrifugation at 50×g to collect the resin. The resin was then washed with 5 volumes of Nickel Wash buffer 3 times, each time using centrifugation to remove excess wash buffer. Bound proteins were then eluted using three washes with Elution Buffer (20 mM HEPES, pH 7.5, 150 mM NaCl, 500 mM imidazole). The eluted protein was concentrated using a 3.5 kDa MWCO centrifugal filter unit (Amicon) before injection onto a Superdex 200 Increase 10/300 GL column equilibrated with 20 mM HEPES, pH 7.5, 150 mM NaCl. Nanobody constructs were concentrated again using a 3.5 k MWCO centrifugal filter unit, and flash frozen in liquid nitrogen.

4. Affinity Determination by Surface Plasmon Resonance

Nanobody (Nb) affinity determination experiments were performed on Biacore T200 and 8K instruments (Cytiva Life Sciences) by capturing the Streptagll-tagged Spike^{S2P} at 10 μg/mL on a StreptactinXT-immobilized (Iba Life Sciences) CM5 Series S sensor chip (Cytiva Life Sciences) to achieve maximum response (Rmax) of approximately 30 response units (RUs) upon nanobody binding. 2-fold serial dilutions of purified nanobody from 1 μM to 31.25 nM (for monovalent constructs) or from 50 nM to 1.56 nM (for affinity matured and multimeric constructs) were flowed over the captured Spike^{S2P} surface at 30 μL/minute for 60 seconds followed by 600 seconds of dissociation flow. Following each cycle, the chip surface was regenerated with 3 M guanidine hydrochloride.

Separately, biotinylated SARS-CoV-2 RBD at 8 μg/mL was loaded onto a preconditioned Series S Sensor Chip CAP chip (Cytiva Life Sciences) to achieve an Rmax of approximately 60 RUs upon nanobody binding. 2-fold serial dilutions in the same running buffer and sample series (parent or affinity matured clone) as the Spike^{S2P} runs were flowed over the RBD surface at 30 μL/minute for 60 seconds followed by 600 seconds of dissociation flow. Chip surface regeneration was performed with a guanidine hydrochloride/sodium hydroxide solution.

The resulting sensorgrams for all monovalent clones were fit to a 1:1 Langmuir binding model using the Biacore Insight Evaluation Software (Cytiva Life Sciences) or the association/dissociation model in GraphPad Prism 8.0. For determination of kinetic parameters for Nb6-bi and Nb6-tri binding, the dissociation phase was fit to a biexponential decay constrained to two dissociation rate constants shared between each concentration. The association phase was fit separately using an association kinetics model simultaneously fitting the association rate constant for each concentration.

For nanobody competition experiments, Spike^{S2P} was loaded onto a StreptactinXT-immobilized CM5 sensor chip as previously described. As in the kinetics experiments, the primary nanobody was flowed over the captured Spike^{S2P} surface for 60 seconds at 30 μL/minute to achieve saturation. Immediately following this, a second injection of a mixture of primary and variable nanobody at the same concentration as in the primary injection was performed.

5. ACE2 Cellular Surface Binding Competition Assays

A dilution series of nanobody was generated in PBE (PBS+0.5% (w/v) BSA+2 mM EDTA and mixed with Spike^{S2P}-Alexa647 or RBD-Alexa647. ACE2 expressing HEK293T cells were dissociated with TrypLE Express (ThermoFisher) and resuspended in PBE(22). The cells were mixed with the Spike^{S2P}-nanobody solution and incubated for 45 minutes, washed in PBE, and then resuspended in PBE. Cell surface Alexa647 fluorescence intensity was assessed on an Attune Flow Cytometer (ThermoFisher).

6. Affinity Maturation of Nb6

A site saturation mutagenesis library of Nb6 was generated by assembly PCR of overlapping oligonucleotides encoding the Nb6 sequence. Individual oligos for each position in CDR1, CDR2, and CDR3 were designed with the degenerate “NNK” codon. The assembled gene product was amplified with oligonucleotides with overlapping ends to enable homologous recombination with the yeast surface display vector as previously described and purified with standard silica-based chromatography(17). The resulting insert DNA was transformed into Saccharomyces cerevisiae strain BJ5465 (ATCC 208289) along with the yeast display vector pYDS2.0 to generate a library of 2×10⁸ transformants. After induction in YPD+NTC medium at 20° C. for 2 days, 2×10⁹ yeast were washed in selection buffer (20 mM HEPES, pH 8.0, 150 mM sodium chloride, 0.1% (w/v) low biotin BSA) and incubated with 1 nM biotin-Spike^{S2P} for 1 hour at 25° C. Yeast were subsequently washed in selection buffer, resuspended in 1 mL selection buffer, and incubated with 10 μL, streptavidin microbeads (Miltenyi) for 15 min. at 4° C. Yeast were washed again with cold selection buffer and Spike^{S2P}-binding yeast were isolated by magnetic separation using an LS column (Miltenyi). Recovered yeast were grown in YPD+NTC at 37° C. and induced in YPG+NTC at 20° C. A second round of selection was performed as above, substituting 100 pM RBD-Alexa647 as the antigen. Yeast displaying high affinity clones were selected by magnetic separation using Anti-Cy5 microbeads (Miltenyi) and an LS column. Analysis of the library after the second round of selection revealed a population of clones with clear binding of 10 pM RBD-Alexa647. Therefore, 96 individual clones were screened for binding to 10 pM RBD-Alexa647 by flow cytometry. Sequence analysis of eight clones that showed robust binding to 10 pM RBD-Alexa647 revealed two consensus mutations, I27Y and P105Y, which were used to generate the affinity matured clone mNb6.

7. mNb6 Crystallography and Structure Determination

Purified mNb6 was concentrated to 18.7 mg/mL and filtered using 0.1 μm hydrophilic PVDF filters (Millipore). mNb6 crystal screens were set up in 96 well plates in hanging drop format at 2:1 protein:reservoir in Index and AmSO4 screens (Hampton Research, Aliso Viejo, Calif.). Crystals in over 60 different screening conditions with various morphologies appeared overnight at ambient temperature and were obtained directly from the screens without further optimization. The crystals were cryoprotected by quick dipping in a solution containing 80% reservoir and 20% PEG400 or 20% Glycerol, then mounted in CrystalCap HT Cryoloops (Hampton Research, Aliso Viejo, Calif.) and flash cooled in a cryogenic nitrogen stream (100 K). All data were collected at the Advanced Light Source (Berkeley, Calif.) beam line 8.3.1. A single crystal of mNb6 that grew in 0.1 M Tris·HCl pH 8.5, 1.0 M Ammonium sulfate diffracted to 2.05 Å. Integration, and scaling were performed with Xia2, using XDS for indexing and integration and XSCALE for scaling and merging(39). The structure was solved molecular replacement using PHASER using the structure of nanobody, Nb.b201 (PDB 5VNV) as search model(17, 40). Model building was performed with COOT and refined with PHENIX and BUSTER(41-43).

6. Structures of Spike-Nanobody Complexes by cryo-EM

A) Sample Preparation and Microscopy

To prepare Spike^{S2P}-nanobody complexes, each nanobody was incubated on ice at a 3-fold molar excess to Spike^{S2P} at 2.5 μM for 10 minutes. 3 μL of Spike^{S2P}-nanobody complex was added to a 300 mesh 1.2/1.3R Au Quantifoil grid previously glow discharged at 15 mA for 30 seconds with a Pelco easiGlow Glow discharge cleaning system. Using Whatman No.1 qualitative filter paper, Blotting was performed with a blot force of 0 for 4 seconds at 4° C. and 100% humidity in a FEI Vitrobot Mark IV (ThermoFisher) prior to plunge freezing into liquid ethane.

For each complex, 120-frame super-resolution movies were collected with a 3×3 image shift collection strategy at a nominal magnification of 105,000× (physical pixel size: 0.834 Å/pix) on a Titan Krios (ThermoFisher) equipped with a K3 camera and a Bioquantum energy filter (Gatan) set to a slit width of 20 eV. Collection dose rate was 8 eipixel/second for a total dose of 66 e⁻/Å². Each collection was performed with semi-automated scripts in SerialEM(44).

B) Image Processing

For all datasets, dose fractionated super-resolution movies were motion corrected with MotionCor2(45). Contrast transfer function determination was performed with cryoSPARC patch CTF(46). Particles were picked with a 20 A low-pass filtered apo Spike 2D templates generated from a prior data collection.

Nb6-Spike^{S2P} and mNb6-Spike^{S2P} particles were extracted with a 384 pixel box, binned to 96 pixels and subject to single rounds of 2D and 3D classification prior to unbinning for homogenous refinement in cryoSPARC. Using pyEM modules, refined particles were then imported into Relion3.1 for 3D classification without alignment using the input refinement map low pass filtered to 40 Å(47, 48). Particles in classes representing the closed conformation of Spike were imported into cisTEM and subject to autorefinement followed by local refinement within a RBD::nanobody masked region(49). Following local refinement, a new refinement package symmetrized to the C3 axis was created for a final round of local refinement without masking. Final particle counts for each map are as follows: Nb6-Open: 40,125, Nb6-Closed: 58,493, mNb6: 53,690.

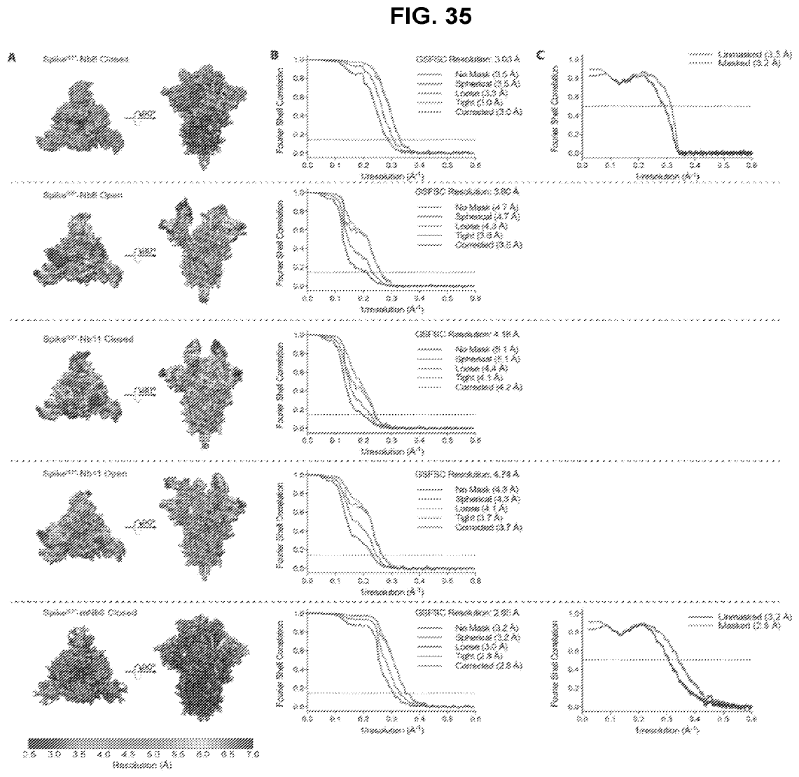

Nb11-Spike^{S2P} particles were extracted with a 512 pixel box, binned to 128 pixels for multiple rounds of 3D classification as described in FIG. 35. Following homogenous refinement, particles were exported to Relion3.1. Particle density roughly corresponding to RBD-nanobody complexes was retained post-particle subtraction. 3D classification without alignment was performed on the particle subtracted stacks. Particles in classes with robust RBD-nanobody density were selected, unsubtracted and refined in Relion followed by post-processing. 21,570 particles contributed to the final maps. Final particle counts for each map are as follows: Nb11-Open: 21,570, Nb11-Closed: 27,611, For all maps, final local resolution estimation and GSFSC determination was carried out in cryoSPARC. Viewing angle distribution plots were generated with pyEM modules and visualized with ChimeraX(50).

C) Structure Modeling

Models of Nb6-Spike^{S2P} and mNb6-Spike^{S2P} were built using a previously determined structure of closed Spike^{S2P} (PDB: 6VXX)(14). A composite model incorporating resolved regions of the RBD was made using a previously determined X-ray crystal structure of the SARS-CoV-2 RBD (PDB: 6M0J)(51). For Nb6, the beta2-adrenergic receptor nanobody Nb80 (PDB: 3P0G) was used as a template to first fit the nanobody into the cryo-EM density map for the Nb6-Spike^{S2P} complex(52). Complementarity determining loops were then truncated and rebuilt using RosettaES(53). The higher resolution structure of mNb6 enabled manual building of nanobody CDR loops de novo, and therefore the Rosetta-based approach was not used for modeling. The final structures were inspected and manually adjusted in COOT and ISOLDE, followed by real space refinement in PHENIX(41, 43, 54) and further refined and relaxed using Rosetta(55). Glycans were refined utilizing the glycan specific Rosetta protocol that incorporates prior knowledge on carbohydrate conformations to ensure lowest energy glycan geometries(56). Final glycan placement was inspected manually and using the Privateer software package distributed under CCP4 (57, 58). Final protein models were analyzed with Molprobity(59), EMRinger(60), PHENIX, with statistics reported in FIG. 42.

For models of Nb11-Spike^{S2P} complexes presented here, the closest nanobody by sequence in the PDB (beta2-adrenergic receptor Nb60, PDB ID: 5JQH) was fit by rigid-body refinement in COOT into the cryo-EM density map using only the framework regions(61). While the lower resolution of these maps precluded confident assignment of loop conformations, the overall orientation of Nb11 relative to Spike^{S2P} was well constrained, enabling accurate modeling of distances between the N- and C-termini of two Nb11 molecules bound to Spike^{S2P}.

Radiolytic hydroxyl radical footprinting and mass-spectrometry of apo and Nb3-bound Spike^{S2P}

Spike^{S2P} and Nb3 samples were buffer exchanged into 10 mM phosphate buffer (pH 7.4) by extensive dialysis at 25° C. A 1.5-fold molar excess of Nb3 was added to 5 μM Spike^{S2P} and the complex was incubated for >24 hr at 25° C. For radiolytic footprinting, protein concentrations and beam parameters were optimized using an Alexa-488 fluorophore assay(l8). Apo Spike^{S2P} and Spike^{S2P}-Nb3 complex at concentrations of 1-3 μM were exposed to a synchrotron X-ray white beam at 6 timepoints between 0-50 ms at beamline 3.2.1 at the Advanced Light Source in Berkeley, Calif. and were quenched with 10 mM methionine amide immediately post-exposure. Glycans were removed by treatment with 5% SDS, 5 mM DTT at 95° C. for five minutes and subsequent PNGase (Promega) digestion at 37° C. for 2 hours. Samples were buffer exchanged into ammonium bicarbonate (ABC) buffer (pH 8.0) using ZebaSpin columns (Thermo Fisher). Alkylation of cysteines was achieved by treatment with 8 M urea and 5 mM DTT at 37° C. for 30 minutes followed by an incubation with 15 mM iodoacetamide at 25° C. in the dark for 30 minutes. All samples were further buffer exchanged to ABC pH 8.0 using ZebaSpin columns and digested with either Trypsin/Lys-C or Glu-C (Promega) at an enzyme:protein ratio of 1:20 (w/w) at 37° C. for 8 hours.

Samples were lyophilized and resuspended in 1% formic acid at 200 fmol/μL concentration. For each MS analysis, 1 μL of sample was injected onto a 5 mm Thermo Trap C18 cartridge, and then separated over a 15 cm column packed with 1.9 μm Reprosil C18 particles (Dr. Maisch HPLC GmbH) by a nanoElute HPLC (Bruker). Separation was performed at 50° C. and a flow rate of 400 μL/min by the following gradient in 0.1% formic acid: 2% to 17% acetonitrile from 0 to 20 min, followed by 17% to 28% acetonitrile from 20 to 40 min. The eluent was electrospray ionized into a Bruker timsTOF Pro mass spectrometer and data was collected using data-dependent PASEF acquisition. Database searching and extraction of MS1 peptide abundances was performed using the FragPipe platform with either trypsin or GluC enzyme specificity, and all peptide and protein identifications were filtered to a 1% false-discovery rate(62). Searches were performed against a concatenated protein database of the Spike protein, common contaminant proteins, and the Saccharomyces cerevisiae proteome (downloaded Jul. 23, 2020). Note, the Saccharomyces cerevisiae proteome was included to generate a sufficient population of true negative identifications for robust false discovery rate estimation of peptide and protein identifications. Lastly, the area under the curve MS1 intensities reported from FragPipe were summarized for each peptide species using MSstats(63).

The peak areas of extracted ion chromatograms and associated side-chain modifications were used to quantify modification at each timepoint. Increasing beamline exposure time decreases the fraction of unmodified peptide and can be represented as a site-specific dose-response plot. The rate of hydroxyl radical reactivity (k_{fp}) is dependent on both the intrinsic reactivity of each residue and its solvent accessibility and was calculated by fitting the dose-response to a pseudo-first order reaction scheme in Graphpad Prism Version 8. The ratio of k_{fp} between apo Spike^{S2P} and the Spike-Nb3 complex at specific residues gave information on solvent accessibility changes between the two samples. These changes were mapped onto the SARS-CoV-2 Spike (PDB 6XR8)(11). In some cases, heavily modified residues show a flattening of dose-response at long exposures which we interpret as radical induced damage. These over-exposed timepoints were excluded from the calculation of kfₚ.

D) Pseudovirus Assays for Nanobody Neutralization

ZsGreen SARS-CoV-2-pseudotyped lentivirus was generated according to a published protocol(22). The day before transduction, 50,000 ACE2 expressing HEK293T cells were plated in each well of a 24-well plate. 10-fold serial dilutions of nanobody were generated in complete medium (DMEM+10% FBS+PSG) and pseudotyped virus was added to a final volume of 200 μL. Media was replaced with nanobody/pseudotyped virus mixture for four hours, then removed. Cells were washed with complete medium and then incubated in complete medium at 37° C. Three days post-transduction, cells were trypsinized and the proportion of ZsGreen+ cells was measured on an Attune flow cytometer (ThermoFisher).

E) Authentic SARS-CoV-2 Neutralization Assay

SARS-CoV-2, isolate France/IDF0372/2020, was supplied by the National Reference Centre for Respiratory Viruses hosted by Institut Pasteur (Paris, France) and headed by Pr. Sylvie van der Werf. Viral stocks were prepared by propagation in Vero E6 cells in Dulbecco's modified Eagle's medium (DMEM) supplemented with 2% (v/v) fetal bovine serum (FBS, Invitrogen). Viral titers were determined by plaque assay. All plaque assays involving live SARS-CoV-2 were performed at Institut Pasteur Paris (IPP) in compliance with IPP's guidelines following Biosafety Level 3 (BSL-3) containment procedures in approved laboratories. All experiments were performed in at least three biologically independent samples.

Neutralization of infectious SARS-CoV-2 was performed using a plaque reduction neutralization test in Vero E6 cells (CRL-1586, ATCC). Briefly, nanobodies (or ACE2-Fc) were eight-fold serially diluted in DMEM containing 2% (v/v) FBS and mixed with 50 plaque forming units (PFU) of SARS-CoV-2 for one hour at 37° C., 5% CO₂. The mixture was then used to inoculate Vero E6 cells seeded in 12-well plates, for one hour at 37° C., 5% CO2. Following this virus adsorption time, a solid agarose overlay (DMEM, 10% (v/v) FBS and 0.8% agarose) was added. The cells were incubated for a further 3 days prior to fixation using 4% formalin and plaques visualized by the addition of crystal violet. The number of plaques in quadruplicate wells for each dilution was used to determine the half maximal inhibitory concentrations (IC₅₀) using 3-parameter logistic regression (GraphPad Prism version 8).

F) Nanobody Stability Studies

Nanobody thermostability by circular dichroism was assessed using a Jasco J710 CD spectrometer equipped with a Peltier temperature control. Individual nanobody constructs were diluted to 5 μM in phosphate buffered saline. Mollar ellipticity was measured at 204 nm (2 nm bandwidth) between 25° C. and 80° C. with a 1° C./min heating rate. The resulting molar ellipticity values were normalized and plotted in GraphPad Prism 8.0 after applying a nearest neighbor smoothing function.

For nanobody competition experiments on ACE2 expressing HEK293T cells, nanobodies were incubated at either 25° C. or 50° C. for one hour. Alternatively, each nanobody was aerosolized with a portable mesh nebulizer producing 2-5 μm particles at a final concentration of 0.5 mg/mL. The resulting aerosol was collected by condensation into a 50 mL tube cooled on ice. Samples were then treated as indicated above to determine IC50 values for binding to Spike^{S2P}-Alexa647 or used for pseudovirus neutralization studies as described above.

Further experiments assessing mNb6 and mNb6-tri stability to aerosolization and lyophilization used a starting concentration of 0.5 mg/mL of each construct. Aerosolization was performed as described above. For lyophilization, nanobodies were first flash frozen in liquid nitrogen and the solution was dried to completion under vacuum. The resulting dried material was resuspended in 20 mM HEPES pH 7.5, 150 mM NaCl. Size exclusion chromatography of the unstressed, post-aerosolization, and post-lyophilization samples were performed an a Superdex 75 Increase 10/300 column in 20 mM HEPES pH 7.5, 150 mM NaCl. SPR experiments to assess binding to Spike^{S2P} were performed as described above. For live SARS-CoV-2 virus experiments, aerosolized, lyophilized, or heat-treated samples were flash frozen in liquid nitrogen prior to shipping.

### 1.5 Example 5

### Evaluation of Nanoparticle A for Treatment of SARS-CoV-2 Infection and Transmission in Golden Syrian Hamsters

Objective: The objective of this study was to assess the efficacy of Nanoparticle A for treatment of a SARS-CoV-2 infection in wild-type golden Syrian hamsters. In addition, the efficacy of Nanoparticle A at reducing the direct transmission of SARS-CoV-2 from infected animals to naïve littermates was also evaluated. The effect of Nanoparticle A treatment on weight loss, lung virus titers, and lung weights in hamsters exposed to SARS-CoV-2 were the primary endpoints.

Materials and Methods:

Animals: Female 5-week-old golden Syrian hamsters were obtained from Charles River Laboratories (Wilmington, Mass.) for this experiment. The hamsters were quarantined for 3 days before use and maintained on Teklad Rodent Diet (Harlan Teklad) and tap water at the Laboratory Animal Research Center of Utah State University.

Virus: Severe Acute Respiratory Syndrome Coronavirus-2 (SARS-CoV-2) USA_WA1/2020 strain was obtained from the World Reference Center for Emerging Viruses and Arboviruses (WRCEVA). The virus was passaged two times in Vero 76 cells to generate a working stock for infection of hamsters.

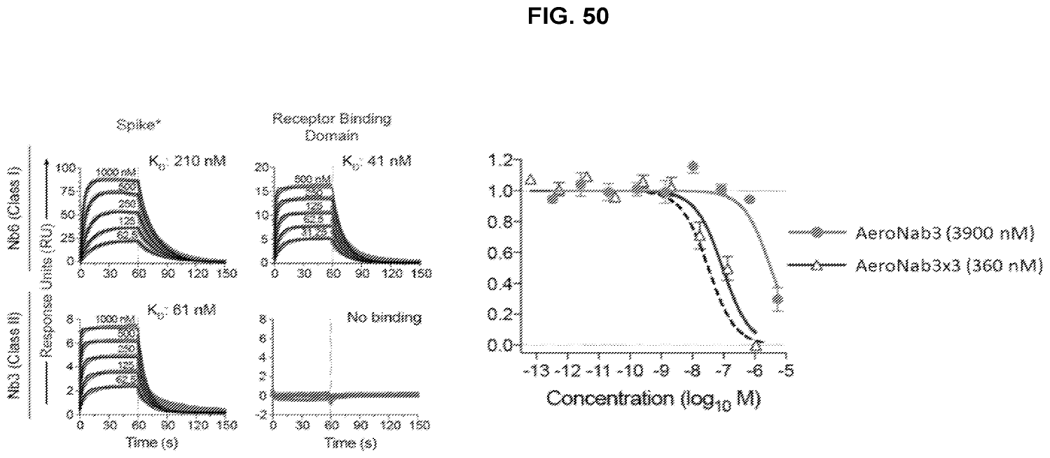

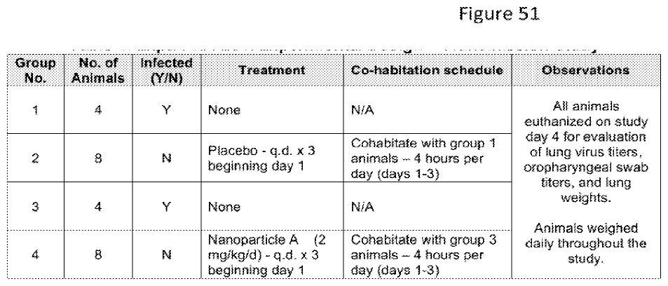

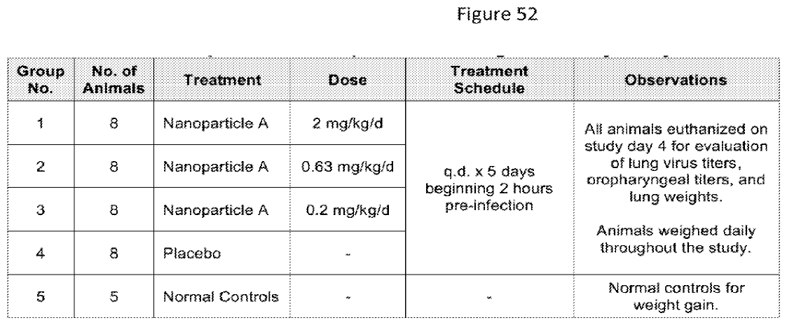

Experiment Design—Transmission and Efficacy Study: A total of 24 5-week-old female golden Syrian hamsters were randomized into 2 groups of 4 hamsters to serve as untreated infected animals and 2 groups of 8 naïve hamsters for cohabitation with and without Nanoparticle A treatment (FIG. 51). For the efficacy study, a total of 37 hamsters were divided into 4 groups of 8 animals per treatment dose with 5 animals used as normal controls for weight gain (FIG. 52). For virus challenge, hamsters in groups 1, 3, and 5 were anesthetized by IP injection of ketamine/xylazine (50 mg/kg/5 mg/kg) prior to challenge by the intranasal route with a dose of 1×104.3 50% cell culture infectious doses (CCID50) in a 100 μl inoculum volume. All intranasal treatments were administered in a 100 μl volume after anesthetizing animals as was done for infections. Animals in groups 1 and 3 were not treated. Animals in group 2 and 4 were not infected but were cohabitated with animals from groups 1 or 3 for 4 hours each day on study days 1, 2, and 3. Animals in group 2 were treated with saline as a placebo. Animals in group 4 were treated once daily with Nanoparticle A 2 hours prior to cohabitation with infected animals. Hamsters were weighed prior to infection and then everyday thereafter to evaluate infection-associated weight loss. All animals were euthanized on study day 4 to evaluate lung virus titers and the transmission of virus from infected animals to naïve animals. Oropharyngeal swabs were collected on all animals.

Titration of Lung Tissue Samples: Lung tissues homogenates were titrated by endpoint dilution. Serial log 10 dilutions of lung tissue homogenate were plated in quadruplicate wells of 96-well microplates containing confluent monolayers of Vero 76 cells. The plates were incubated in at 37° C. incubator with 5% CO2 for 6 days. The plates were then scored by visual observation under a light microscope for the presence of cytopathic effect (CPE). Virus titer for each sample was calculated by linear regression using the Reed-Muench method (Reed L J and Muench H, A simple method of estimating fifty per cent endpoints. American Journal of Hygiene. 1938).

Statistics and Figures: Individual hamster body weights were converted to a percent of initial body weight on the day of infection. The percentage of initial body weight curves were compared using a one-way analysis of variance (ANOVA) comparing each treatment group to placebo-treated hamsters. Lung virus titers and lung weights were compared using a one-way ANOVA comparing the treated animals to placebo-treated animals.

This study evaluated the efficacy of intranasal treatment with Nanoparticle A on the transmission of SARS-CoV-2 in golden Syrian hamsters. Treatment with Nanoparticle A was also evaluated for reduction of lung virus titers and lung weights in hamsters infected with SARS-CoV-2.

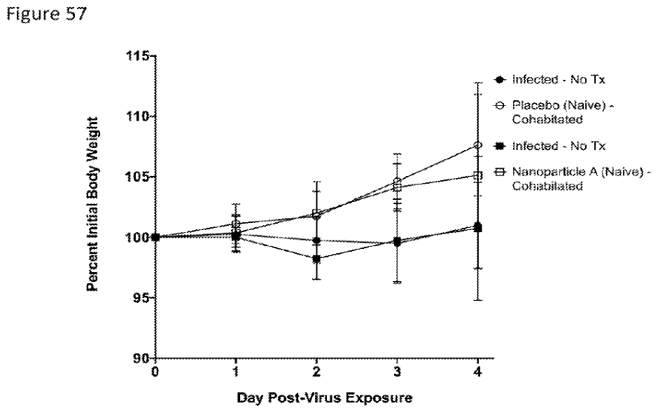

Results: Percent initial body weight of 5-week-old golden Syrian hamsters following challenge with SARS-CoV-2 and treatment with Nanoparticle A prior to cohabitation with infected animals is shown in FIG. 57. Animals with the same shape symbols were cohabitated. Groups represented with the closed circle and closed square were infected on study day 0. Animals represented by the open circle were naïve and placebo-treated prior to cohabitation with animals from group 1 for 4 hrs per day for 3 days. Animals represented by the open square were naïve and Nanoparticle A-treated prior to cohabitation with animals from group 3. The differences in percent initial body weight were not statistically significant when compared by one-way ANOVA.

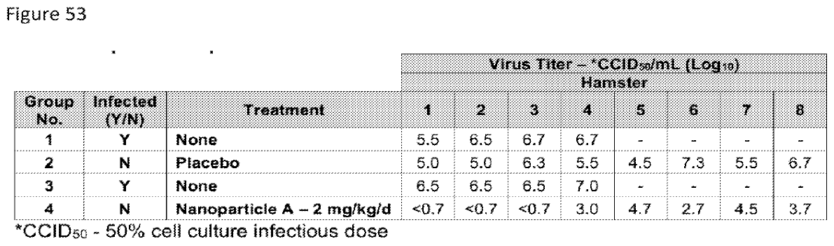

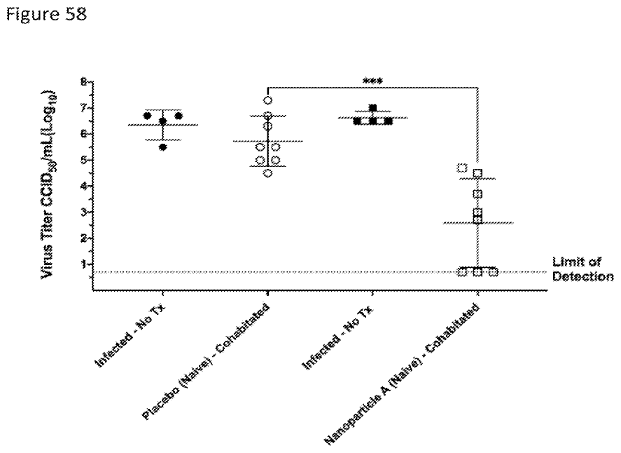

FIG. 58 shows lung virus titers of 5-week-old golden Syrian hamsters after challenge with SARS-CoV-2 and treatment with Nanoparticle A prior to cohabitation with infected animals. Animals with the same shape symbols were cohabitated. Groups represented with the closed circle and closed square were infected on study day 0. Animals represented by the open circle were naïve and placebo-treated prior to cohabitation with animals from group 1 for 4 hrs per day for 3 days. Animals represented by the open square were naïve and Nanoparticle A-treated prior to cohabitation with animals from group 3. Treatment with Nanoparticle A significantly reduced lung virus titers in naïve animals cohabitated with SARS-CoV-2-infected animals. This data is summarized in FIG. 53.

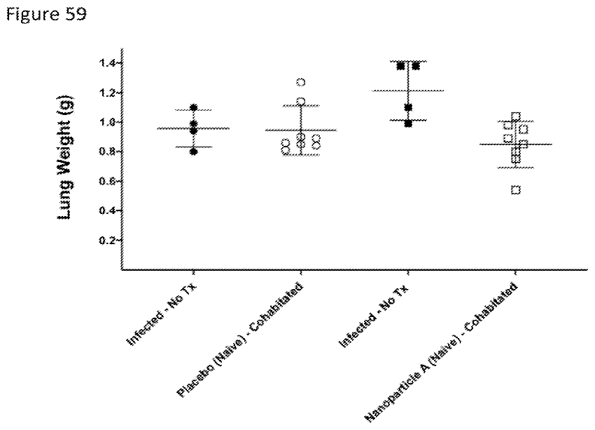

FIG. 59 shows lung weights of 5-week-old golden Syrian hamsters after challenge with SARS-CoV-2 and treatment with Nanoparticle A prior to cohabitation with infected animals. Animals with the same shape symbols were cohabitated. Groups represented with the closed circle and closed square were infected on study day 0. Animals represented by the open circle were naïve and placebo-treated prior to cohabitation with animals from group 1 for 4 hrs per day for 3 days. Animals represented by the open square were naïve and Nanoparticle A-treated prior to cohabitation with animals from group 3. Lung weights were not statistically different between groups when compared by one-way ANOVA.

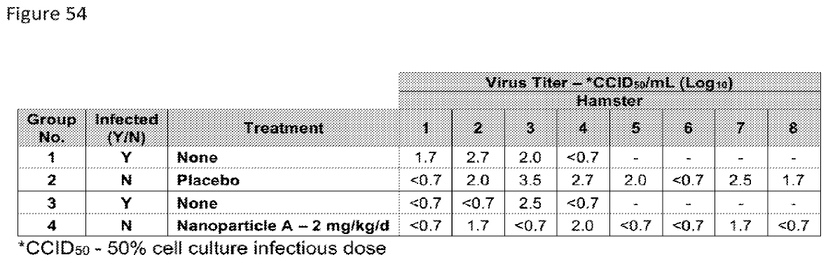

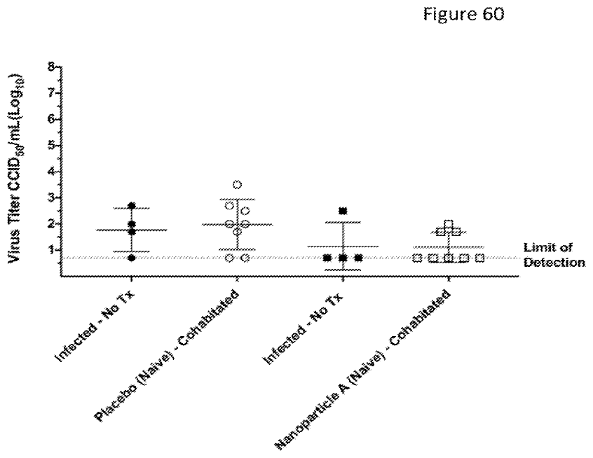

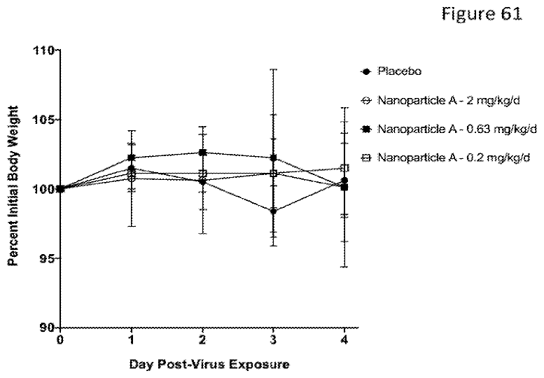

Oropharyngeal swab virus titers of 5-week-old golden Syrian hamsters after challenge with SARS-CoV-2 and treatment with Nanoparticle A prior to cohabitation with infected animals are shown in FIG. 60. Animals with the same shape symbols were cohabitated. Groups represented with the closed circle and closed square were infected on study day 0. Animals represented by the open circle were naïve and placebo-treated prior to cohabitation with animals from group 1 for 4 hrs per day for 3 days. Animals represented by the open square were naïve and Nanoparticle A-treated prior to cohabitation with animals from group 3. No significant difference in oropharyngeal swab virus titers were determined by one-way ANOVA. This data is summarized in FIG. 54 and FIG. 61 shows percent initial body weight of 5-week-old golden Syrian hamsters following treatment with Nanoparticle A and infection with SARS-CoV-2. The differences in percent initial body weight were not statistically significant when compared by one-way ANOVA.

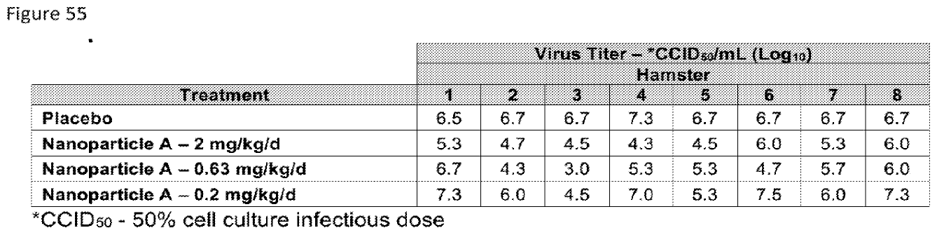

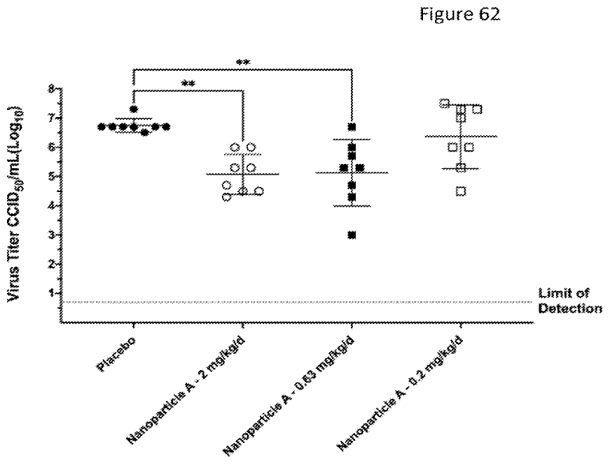

FIG. 62 shows lung virus titers of 5-week-old golden Syrian hamsters after treatment with Nanoparticle A and infection with SARS-CoV-2. Treatment with Nanoparticle A started significantly reduced lung virus titers at doses of 2 and 0.63 mg/kg/d compared to placebo-treated animals. This data is summarized in FIG. 55.

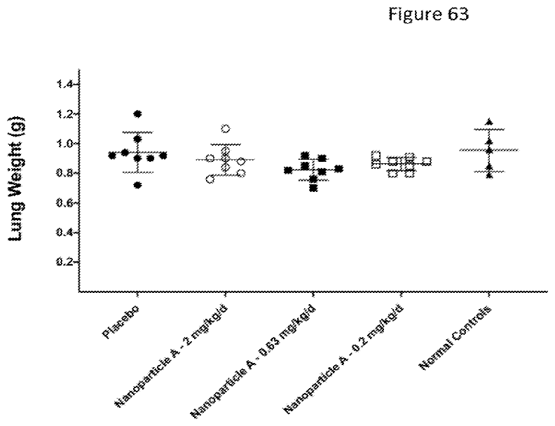

FIG. 63 shows lung weights of 5-week-old golden Syrian hamsters after challenge with SARS-CoV-2 and treatment with Nanoparticle A prior to cohabitation with infected animals. Lung weights were not statistically different between groups when compared by one-way ANOVA.

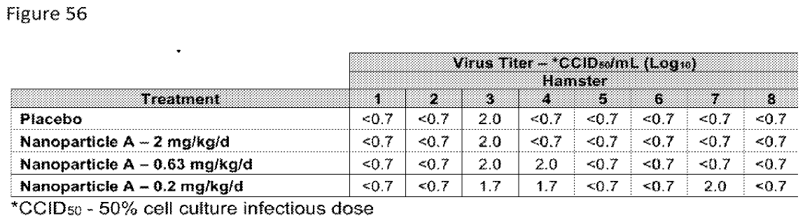

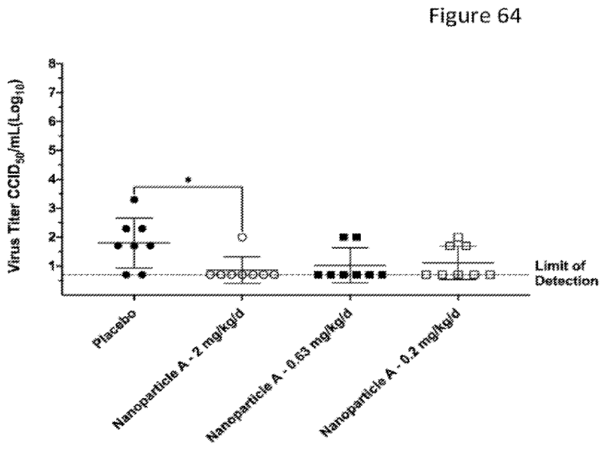

Oropharyngeal swab virus titers of 5-week-old golden Syrian hamsters after treatment with Nanoparticle A and infection with SARS-CoV-2 are shown in FIG. 64. Treatment with Nanoparticle A at a dose of 2 mg/kg/d significantly reduced oropharyngeal swab titers of hamsters infected with SARS-CoV-2. This data is summarized in FIG. 56.

Conclusion: This study evaluated the efficacy of intranasal Nanoparticle A treatment on the transmission of SARS-CoV-2 infection in golden Syrian hamsters. The impact of treatment with Nanoparticle A on lung virus titers, oropharyngeal swab titers, and lung weights was also evaluated. Treatment with Nanoparticle A at a dose of 2 mg/kg/d significantly reduced transmission of SARS-CoV-2 to naïve animals. The virus was not detected in the lungs of three of the eight naïve animals that were exposed to infected animals. In the remaining five animals, lung virus titers were reduced by at least one log 10 compared to placebo-treated naïve animals. Oropharyngeal swab titers were significantly reduced by treatment with Nanoparticle A although the virus was not consistently detected even in placebo-treated animals. In the efficacy study, lung virus titers were significantly reduced lung virus titers in animals treated with 2 or 0.63 mg/kg/d compared to placebo-treated animals. Oropharyngeal titers were also significantly reduced by a 2 mg/kg/d dose of Nanoparticle A compared to placebo-treated animals. Oropharyngeal swab titers were only detected in one of eight, two of eight, and three of eight animals at doses of 2, 0.63, and 0.2 mg/kg/d respectively. Oropharyngeal swab titers were detected in six of eight placebo-treated animals.

No adverse reactions to treatment were observed in any of the animals. A lack of weight loss following treatment also indicates that the treatment was well-tolerated in hamsters.

### REFERENCES AND NOTES

1. T. G. Ksiazek et al., A novel coronavirus associated with severe acute respiratory syndrome. N Engl J Med 348, 1953-1966 (2003).

2. A. M. Zaki, S. van Boheemen, T. M. Bestebroer, A. D. Osterhaus, R. A. Fouchier, Isolation of a novel coronavirus from a man with pneumonia in Saudi Arabia. N Engl J Med 367, 1814-1820 (2012).

3. P. Zhou et at, A pneumonia outbreak associated with a new coronavirus of probable bat origin. Nature 579, 270-273 (2020).

4. J. F. Chan et al., A familial cluster of pneumonia associated with the 2019 novel coronavirus indicating person-to-person transmission: a study of a family cluster. Lancet 395, 514-523 (2020).

5. C. Huang et al., Clinical features of patients infected with 2019 novel coronavirus in Wuhan, China. Lancet 395, 497-506 (2020).

6. F. Wu et al., A new coronavirus associated with human respiratory disease in China. Nature 579, 265-269 (2020).

7. N. Zhu et al., A Novel Coronavirus from Patients with Pneumonia in China, 2019. N Engl J Med 382, 727-733 (2020).

8. Z. Ke et al., Structures and distributions of SARS-CoV-2 spike proteins on intact virions. Nature, (2020).

9. B. Turonova et al., In situ structural analysis of SARS-CoV-2 spike reveals flexibility mediated by three hinges. Science 370, 203-208 (2020).

10. B. J. Bosch, R. van der Zee, C. A. de Haan, P. J. Rottier, The coronavirus spike protein is a class I virus fusion protein: structural and functional characterization of the fusion core complex. J Virol 77, 8801-8811 (2003).

11. Y. Cai et al., Distinct conformational states of SARS-CoV-2 spike protein. Science 369, 1586-1592 (2020).

12. Q. Wang et al., Structural and Functional Basis of SARS-CoV-2 Entry by Using Human ACE2. Cell 181, 894-904 e899 (2020).

13. R. Yan et al., Structural basis for the recognition of SARS-CoV-2 by full-length human ACE2. Science 367, 1444-1448 (2020).

14. A. C. Walls et al., Structure, Function, and Antigenicity of the SARS-CoV-2 Spike Glycoprotein. Cell 181, 281-292 e286 (2020).

15. D. Wrapp et al., Cryo-EM structure of the 2019-nCoV spike in the prefusion conformation. Science 367, 1260-+(2020).

16. M. Hoffmann et al., SARS-CoV-2 Cell Entry Depends on ACE2 and TMPRSS2 and Is Blocked by a Clinically Proven Protease Inhibitor. Cell 181, 271-280 e278 (2020).

17. C. McMahon et al., Yeast surface display platform for rapid discovery of conformationally selective nanobodies. Nat Struct Mol Biol 25, 289-296 (2018).

18. S. Gupta, M. Sullivan, J. Toomey, J. Kiselar, M. R. Chance, The Beamline X28C of the Center for Synchrotron Biosciences: a national resource for biomolecular structure and dynamics experiments using synchrotron footprinting. J Synchrotron Radiat 14, 233-243 (2007).

19. Y. Zhang, A. T. Wecksler, P. Molina, G. Deperalta, M. L. Gross, Mapping the Binding Interface of VEGF and a Monoclonal Antibody Fab-1 Fragment with Fast Photochemical Oxidation of Proteins (FPOP) and Mass Spectrometry. J Am Soc Mass Spectrom 28, 850-858 (2017).

20. X. Chi et al., A neutralizing human antibody binds to the N-terminal domain of the Spike protein of SARS-CoV-2. Science 369, 650-655 (2020).

21. H. Zhou et al., Structural definition of a neutralization epitope on the N-terminal domain of MERS-CoV spike glycoprotein. Nat Commun 10, 3068 (2019).

22. K. H. D. Crawford et al., Protocol and Reagents for Pseudotyping Lentiviral Particles with SARS-CoV-2 Spike Protein for Neutralization Assays. Viruses 12, (2020).

23. A. Baum et al., Antibody cocktail to SARS-CoV-2 spike protein prevents rapid mutational escape seen with individual antibodies. Science 369, 1014-1018 (2020).

24. Y. L. Cao et al., Potent Neutralizing Antibodies against SARS-CoV-2 Identified by High-Throughput Single-Cell Sequencing of Convalescent Patients' B Cells. Cell 182, 73-+(2020).

25. B. Ju et al., Human neutralizing antibodies elicited by SARS-CoV-2 infection. Nature 584, 115-119 (2020).

26. L. Liu et al., Potent neutralizing antibodies against multiple epitopes on SARS-CoV-2 spike. Nature 584, 450-456 (2020).

27. D. Pinto et al., Cross-neutralization of SARS-CoV-2 by a human monoclonal SARS-CoV antibody. Nature 583, 290-295 (2020).

28. T. F. Rogers et al., Isolation of potent SARS-CoV-2 neutralizing antibodies and protection from disease in a small animal model. Science 369, 956-963 (2020).

29. S. J. Zost et al., Potently neutralizing and protective human antibodies against SARS-CoV-2. Nature 584, 443-449 (2020).

30. M. A. Tortorici et al., Ultrapotent human antibodies protect against SARS-CoV-2 challenge via multiple mechanisms. Science, (2020).

31. H. Ledford, Antibody therapies could be a bridge to a coronavirus vaccine—but will the world benefit? Nature 584, 333-334 (2020).

32. V. H. Leyva-Grado, G. S. Tan, P. E. Leon, M. Yondola, P. Palese, Direct Administration in the Respiratory Tract Improves Efficacy of Broadly Neutralizing Anti-Influenza Virus Monoclonal Antibodies. Antimicrobial Agents and Chemotherapy 59, 4162-4172 (2015).

33. L. Detalle et al., Generation and Characterization of ALX-0171, a Potent Novel Therapeutic Nanobody for the Treatment of Respiratory Syncytial Virus Infection. Antimicrob Agents Chemother 60, 6-13 (2016).

34. S. Cunningham et al., Nebulised ALX-0171 for respiratory syncytial virus lower respiratory tract infection in hospitalised children: a double-blind, randomised, placebo-controlled, phase 2b trial. Lancet Respir Med, (2020).

35. C. Vincke et al., General strategy to humanize a camelid single-domain antibody and identification of a universal humanized nanobody scaffold. J Biol Chem 284, 3273-3284 (2009).

36. D. Wrapp et al., Structural Basis for Potent Neutralization of Betacoronaviruses by Single-Domain Camelid Antibodies. Cell 181, 1004-1015 e1015 (2020).

37. D. Stadlbauer et at, SARS-CoV-2 Seroconversion in Humans: A Detailed Protocol for a Serological Assay, Antigen Production, and Test Setup. Curr Protoc Microbiol 57, e100 (2020).

38. I. Lui et al., Trimeric SARS-CoV-2 Spike interacts with dimeric ACE2 with limited intra-Spike avidity. bioRxiv, 2020.2005.2021.109157 (2020).

39. W. Kabsch, Automatic Processing of Rotation Diffraction Data from Crystals of Initially Unknown Symmetry and Cell Constants. J Appl Crystallogr 26, 795-800 (1993).

40. A. J. McCoy et at, Phaser crystallographic software. J Appl Crystallogr 40, 658-674 (2007).

41. P. D. Adams et at, PHENIX: a comprehensive Python-based system for macromolecular structure solution. Acta Crystallogr D Biol Crystallogr 66, 213-221 (2010).

42. B. E. Bricogne G., Brandl M., Flensburg C., Keller P., Paciorek W., S. A. Roversi P, Smart O. S., Vonrhein C., Womack T. O., BUSTER version 1.10.0. Cambridge, United Kingdom: Global Phasing Ltd., (2017).

43. P. Emsley, K. Cowtan, Coot: model-building tools for molecular graphics. Acta Crystallogr D Biol Crystallogr 60, 2126-2132 (2004).

44. D. N. Mastronarde, Automated electron microscope tomography using robust prediction of specimen movements. J Struct Biol 152, 36-51 (2005).

45. S. Q. Zheng et al., MotionCor2: anisotropic correction of beam-induced motion for improved cryo-electron microscopy. Nat Methods 14, 331-332 (2017).

46. A. Punjani, J. L. Rubinstein, D. J. Fleet, M. A. Brubaker, cryoSPARC: algorithms for rapid unsupervised cryo-EM structure determination. Nat Methods 14, 290-296 (2017).

47. J. Zivanov et at, New tools for automated high-resolution cryo-EM structure determination in RELION-3. Elife 7, (2018).

48. D. Asarnow, E. Palovcak, Y. Cheng, UCSF pyem v0.5. (2019).

49. T. Grant, A. Rohou, N. Grigorieff, cisTEM, user-friendly software for single-particle image processing. Elife 7, (2018).

50. T. D. Goddard et al., UCSF ChimeraX: Meeting modern challenges in visualization and analysis. Protein Sci 27, 14-25 (2018).

51. J. Lan et al., Structure of the SARS-CoV-2 spike receptor-binding domain bound to the ACE2 receptor. Nature 581, 215-220 (2020).

52. S. G. Rasmussen et al., Structure of a nanobody-stabilized active state of the beta(2) adrenoceptor. Nature 469, 175-180 (2011).

53. B. Frenz, A. C. Walls, E. H. Egelman, D. Veesler, F. DiMaio, RosettaES: a sampling strategy enabling automated interpretation of difficult cryo-EM maps. Nat Methods 14, 797-800 (2017).

54. T. I. Croll, ISOLDE: a physically realistic environment for model building into low-resolution electron-density maps. Acta Crystallogr D Struct Biol 74, 519-530 (2018).

55. R. Y. Wang et al., Automated structure refinement of macromolecular assemblies from cryo-EM maps using Rosetta. Elife 5, (2016).

56. B. Frenz et al., Automatically Fixing Errors in Glycoprotein Structures with Rosetta. Structure 27, 134-139 e133 (2019).

57. J. Agirre et al., Privateer: software for the conformational validation of carbohydrate structures. Nat Struct Mol Biol 22, 833-834 (2015).

58. M. D. Winn et al., Overview of the CCP4 suite and current developments. Acta Crystallogr D Biol Crystallogr 67, 235-242 (2011).

59. V. B. Chen et al., MolProbity: all-atom structure validation for macromolecular crystallography. Acta Crystallogr D Biol Crystallogr 66, 12-21 (2010).

60. B. A. Barad et al., EMRinger: side chain-directed model and map validation for 3D cryo-electron microscopy. Nat Methods 12, 943-946 (2015).

61. D. P. Staus et al., Allosteric nanobodies reveal the dynamic range and diverse mechanisms of G-protein-coupled receptor activation. Nature 535, 448-452 (2016).

62. F. Yu et al., Fast Quantitative Analysis of timsTOF PASEF Data with MSFragger and IonQuant. Mol Cell Proteomics 19, 1575-1585 (2020).

63. M. Choi et al., MSstats: an R package for statistical analysis of quantitative mass spectrometry-based proteomic experiments. Bioinformatics 30, 2524-2526 (2014).

All publications, patent applications, and accession numbers mentioned in this specification are herein incorporated by reference to the same extent as if each individual publication or patent application was specifically and individually indicated to be incorporated by reference for the material for which it is cited.

What is claimed is:

1. A composition comprising a single domain antigen binding domain (sdABD) that binds to the Spike protein having SEQ ID NO:300 or the sequence described in FIG. 17A, wherein said sdABD comprises, from N- to C-terminal, FR1-vhCDR1-FR2-vhCDR2-FR3-vhCDR3-FR, wherein said vhhCDR1, vhhCDR2 and said vhhCDR3 is selected from the sets depicted in FIG. 13, FIG. 15 and FIG. 25.

2. A composition according to claim 1 wherein said vhCDR1 has a sequence GI(I/Y/W/F/V/L)FGRNA, said vhCDR2 has a sequence TRR(G/H/Y/G/Q)SITY and said vhCDR3 has a sequence AADPA(S/V/L/I/T)PA(P/F/W/Y/L/V)GDY.

3. A composition according to claim 1 wherein said vhCDR1 has SEQ ID NO:5, said vhCDR2 has SEQ ID NO:6, and vhCDR3 has SEQ ID NO:7.

4. A composition according to claim 1 wherein said vhCDR1 has SEQ ID NO:8, said vhCDR2 has SEQ ID NO:9, and vhCDR3 has SEQ ID NO:10.

5. A composition according to claim 1 wherein said vhCDR1 has SEQ ID NO:11, said vhCDR2 has SEQ ID NO:12, and vhCDR3 has SEQ ID NO:13.

6. A composition according to claim 1 wherein said vhCDR1 has SEQ ID NO:14, said vhCDR2 has SEQ ID NO:15, and vhCDR3 has SEQ ID NO:16.

7. A composition according to claim 1 wherein said vhCDR1 has SEQ ID NO:17, said vhCDR2 has SEQ ID NO:18, and vhCDR3 has SEQ ID NO:19.

8. A composition according to claim 1 wherein said vhCDR1 has SEQ ID NO:20, said vhCDR2 has SEQ ID NO:21, and vhCDR3 has SEQ ID NO:22.

9. A composition according to claim 1 wherein said vhCDR1 has SEQ ID NO:23, said vhCDR2 has SEQ ID NO:24, and vhCDR3 has SEQ ID NO:25.

10. A composition according to claim 1 wherein said vhCDR1 has SEQ ID NO:26, said vhCDR2 has SEQ ID NO:27, and vhCDR3 has SEQ ID NO:28.

11. A composition according to claim 1 wherein said vhCDR1 has SEQ ID NO:29, said vhCDR2 has SEQ ID NO:30, and vhCDR3 has SEQ ID NO:31.

12. A composition according to claim 1 wherein said vhCDR1 has SEQ ID NO:32, said vhCDR2 has SEQ ID NO:33, and vhCDR3 has SEQ ID NO:34.

13. A composition according to claim 1 wherein said vhCDR1 has SEQ ID NO:35, said vhCDR2 has SEQ ID NO:36, and vhCDR3 has SEQ ID NO:37.

14. A composition according to claim 1 wherein said vhCDR1 has SEQ ID NO:38, said vhCDR2 has SEQ ID NO:39, and vhCDR3 has SEQ ID NO:40.

15. A composition according to claim 1 wherein said vhCDR1 has SEQ ID NO:41, said vhCDR2 has SEQ ID NO:42, and vhCDR3 has SEQ ID NO:43.

16. A composition according to claim 1 wherein said vhCDR1 has SEQ ID NO44:, said vhCDR2 has SEQ ID NO45:, and vhCDR3 has SEQ ID NO:46.

17. A composition according to claim 1 wherein said vhCDR1 has SEQ ID NO:47, said vhCDR2 has SEQ ID NO:48, and vhCDR3 has SEQ ID NO:49.

18. A composition according to claim 1 wherein said vhCDR1 has SEQ ID NO:50, said vhCDR2 has SEQ ID NO:51, and vhCDR3 has SEQ ID NO:52.

19. A composition according to claim 1 wherein said vhCDR1 has SEQ ID NO:53, said vhCDR2 has SEQ ID NO:54, and vhCDR3 has SEQ ID NO:55.

20. A composition according to claim 1 wherein said vhCDR1 has SEQ ID NO:56, said vhCDR2 has SEQ ID NO:57, and vhCDR3 has SEQ ID NO:58.

21. A composition according to claim 1 wherein said vhCDR1 has SEQ ID NO:59, said vhCDR2 has SEQ ID NO:60, and vhCDR3 has SEQ ID NO:61.

22. A composition according to claim 1 wherein said vhCDR1 has SEQ ID N062 said vhCDR2 has SEQ ID NO:63, and vhCDR3 has SEQ ID NO:64.

23. A composition according to claim 1 wherein said vhCDR1 has SEQ ID NO:65, said vhCDR2 has SEQ ID NO:66, and vhCDR3 has SEQ ID NO:67.

24. A composition according to claim 1 wherein said vhCDR1 has SEQ ID NO:68, said vhCDR2 has SEQ ID NO:69, and vhCDR3 has SEQ ID NO70:.

25. A composition according to claim 1 wherein said vhCDR1 has SEQ ID NO:71, said vhCDR2 has SEQ ID NO:72, and vhCDR3 has SEQ ID NO:73.

26. A composition according to claim 1 wherein said vhCDR1 has SEQ ID NO:74, said vhCDR2 has SEQ ID NO:75, and vhCDR3 has SEQ ID NO:76.

27. A composition according to any of claims 3 to 25 wherein the framework sequences of said sdABD have SEQ ID NO:1, SEQ ID NO:2, SEQ ID NO:3 and SEQ ID NO:4.

28. A composition according to any of claims 1 to 25 wherein said composition further comprises an half-life extension domain covalently attached to said sdABD using a domain linker.

29. A composition according to claim 26 wherein said half-life extension domain is selected from the group consisting of an anti-human serum albumin (HSA) sdABD and all or part of HSA.

30. A composition comprising a MASC fusion protein that binds to the Spike protein having SEQ ID NO: 300 or the sequence described in FIG. 17A, wherein said MASC fusion protein comprises, from N- to C-terminal, FR1-vhCDR1-FR2-vhCDR2-FR3-vhCDR3-FR-domain linker-FR1-vhCDR1-FR2-vhCDR2-FR3-vhCDR3-FR, wherein said vhhCDR1, vhhCDR2 and said vhhCDR3 is selected from the sets depicted in FIG. 13, FIG. 15 and FIG. 25.

31. A composition according to claim 30 wherein said vhCDR1 has a sequence GI(I/Y/W/F/V/L)FGRNA, said vhCDR2 has a sequence TRR(G/H/Y/G/Q)SITY and said vhCDR3 has a sequence AADPA(S/V/L/I/T)PA(P/F/W/Y/L/V)GDY.

32. A composition according to claim 30 wherein said vhCDR1 has SEQ ID NO:5, said vhCDR2 has SEQ ID NO:6, and vhCDR3 has SEQ ID NO:7.

33. A composition according to claim 30 wherein said vhCDR1 has SEQ ID NO:8, said vhCDR2 has SEQ ID NO:9, and vhCDR3 has SEQ ID NO:10.

34. A composition according to claim 30 wherein said vhCDR1 has SEQ ID NO:11, said vhCDR2 has SEQ ID NO:12, and vhCDR3 has SEQ ID NO:13.

35. A composition according to claim 30 wherein said vhCDR1 has SEQ ID NO:14, said vhCDR2 has SEQ ID NO:15, and vhCDR3 has SEQ ID NO:16.

36. A composition according to claim 30 wherein said vhCDR1 has SEQ ID NO:17, said vhCDR2 has SEQ ID NO:18, and vhCDR3 has SEQ ID NO:19.

37. A composition according to claim 30 wherein said vhCDR1 has SEQ ID NO:20, said vhCDR2 has SEQ ID NO:21, and vhCDR3 has SEQ ID NO:22.

38. A composition according to claim 30 wherein said vhCDR1 has SEQ ID NO:23, said vhCDR2 has SEQ ID NO:24, and vhCDR3 has SEQ ID NO:25.

39. A composition according to claim 30 wherein said vhCDR1 has SEQ ID NO:26, said vhCDR2 has SEQ ID NO:27, and vhCDR3 has SEQ ID NO:28.

40. A composition according to claim 30 wherein said vhCDR1 has SEQ ID NO:29, said vhCDR2 has SEQ ID NO:30, and vhCDR3 has SEQ ID NO:31.

41. A composition according to claim 30 wherein said vhCDR1 has SEQ ID NO:32, said vhCDR2 has SEQ ID NO:33, and vhCDR3 has SEQ ID NO:34.

42. A composition according to claim 30 wherein said vhCDR1 has SEQ ID NO:35, said vhCDR2 has SEQ ID NO:36, and vhCDR3 has SEQ ID NO:37.

43. A composition according to claim 30 wherein said vhCDR1 has SEQ ID NO:38, said vhCDR2 has SEQ ID NO:39, and vhCDR3 has SEQ ID NO:40.

44. A composition according to claim 30 wherein said vhCDR1 has SEQ ID NO:41, said vhCDR2 has SEQ ID NO:42, and vhCDR3 has SEQ ID NO:43.

45. A composition according to claim 30 wherein said vhCDR1 has SEQ ID NO44:, said vhCDR2 has SEQ ID NO45:, and vhCDR3 has SEQ ID NO:46.

46. A composition according to claim 30 wherein said vhCDR1 has SEQ ID NO:47, said vhCDR2 has SEQ ID NO:48, and vhCDR3 has SEQ ID NO:49.

47. A composition according to claim 30 wherein said vhCDR1 has SEQ ID NO:50, said vhCDR2 has SEQ ID NO:51, and vhCDR3 has SEQ ID NO:52.

48. A composition according to claim 30 wherein said vhCDR1 has SEQ ID NO:53, said vhCDR2 has SEQ ID NO:54, and vhCDR3 has SEQ ID NO:55.

49. A composition according to claim 30 wherein said vhCDR1 has SEQ ID NO:56, said vhCDR2 has SEQ ID NO:57, and vhCDR3 has SEQ ID NO:58.

50. A composition according to claim 30 wherein said vhCDR1 has SEQ ID NO:59, said vhCDR2 has SEQ ID NO:60, and vhCDR3 has SEQ ID NO:61.

51. A composition according to claim 30 wherein said vhCDR1 has SEQ ID NO62 said vhCDR2 has SEQ ID NO:63, and vhCDR3 has SEQ ID NO:64.

52. A composition according to claim 30 wherein said vhCDR1 has SEQ ID NO:65, said vhCDR2 has SEQ ID NO:66, and vhCDR3 has SEQ ID NO:67.

53. A composition according to claim 30 wherein said vhCDR1 has SEQ ID NO:68, said vhCDR2 has SEQ ID NO:69, and vhCDR3 has SEQ ID NO70:.

54. A composition according to claim 30 wherein said vhCDR1 has SEQ ID NO:71, said vhCDR2 has SEQ ID NO:72, and vhCDR3 has SEQ ID NO:73.

55. A composition according to claim 30 wherein said vhCDR1 has SEQ ID NO:74, said vhCDR2 has SEQ ID NO:75, and vhCDR3 has SEQ ID NO:76.

56. A composition according to any of claims 32 to 54 wherein the framework sequences of said sdABD have SEQ ID NO:1, SEQ ID NO:2, SEQ ID NO:3 and SEQ ID NO:4.

57. A composition according to any of claims 30 to 54 wherein said composition further comprises an half-life extension domain covalently attached to said sdABD using a domain linker.

58. A composition according to claim 55 wherein said half-life extension domain is selected from the group consisting of an anti-human serum albumin (HSA) sdABD and all or part of HSA.

59. A composition comprising a MASC fusion protein that binds to the Spike protein having SEQ ID NO: 300 or the sequence described in FIG. 17A, wherein said MASC fusion protein comprises, from N- to C-terminal, FR1-vhCDR1-FR2-vhCDR2-FR3-vhCDR3-FR-domain linker-FR1-vhCDR1-FR2-vhCDR2-FR3-vhCDR3-FR-domain linker-FR1-vhCDR1-FR2-vhCDR2-FR3-vhCDR3-FR, wherein said vhhCDR1, vhhCDR2 and said vhhCDR3 is selected from the sets depicted in FIG. 13, FIG. 15 and FIG. 25.

60. A composition according to claim 59 wherein said vhCDR1 has a sequence GI(I/Y/W/F/V/L)FGRNA, said vhCDR2 has a sequence TRR(G/H/Y/G/Q)SITY and said vhCDR3 has a sequence AADPA(S/V/L/I/T)PA(P/F/W/Y/L/V)GDY.

61. A composition according to claim 59 wherein said vhCDR1 has SEQ ID NO:5, said vhCDR2 has SEQ ID NO:6, and vhCDR3 has SEQ ID NO:7.

62. A composition according to claim 59 wherein said vhCDR1 has SEQ ID NO:8, said vhCDR2 has SEQ ID NO:9, and vhCDR3 has SEQ ID NO:10.

63. A composition according to claim 59 wherein said vhCDR1 has SEQ ID NO:11, said vhCDR2 has SEQ ID NO:12, and vhCDR3 has SEQ ID NO:13.

64. A composition according to claim 59 wherein said vhCDR1 has SEQ ID NO:14, said vhCDR2 has SEQ ID NO:15, and vhCDR3 has SEQ ID NO:16.

65. A composition according to claim 59 wherein said vhCDR1 has SEQ ID NO:17, said vhCDR2 has SEQ ID NO:18, and vhCDR3 has SEQ ID NO:19.

66. A composition according to claim 59 wherein said vhCDR1 has SEQ ID NO:20, said vhCDR2 has SEQ ID NO:21, and vhCDR3 has SEQ ID NO:22.

67. A composition according to claim 59 wherein said vhCDR1 has SEQ ID NO:23, said vhCDR2 has SEQ ID NO:24, and vhCDR3 has SEQ ID NO:25.

68. A composition according to claim 59 wherein said vhCDR1 has SEQ ID NO:26, said vhCDR2 has SEQ ID NO:27, and vhCDR3 has SEQ ID NO:28.

69. A composition according to claim 59 wherein said vhCDR1 has SEQ ID NO:29, said vhCDR2 has SEQ ID NO:30, and vhCDR3 has SEQ ID NO:31.

70. A composition according to claim 59 wherein said vhCDR1 has SEQ ID NO:32, said vhCDR2 has SEQ ID NO:33, and vhCDR3 has SEQ ID NO:34.

71. A composition according to claim 59 wherein said vhCDR1 has SEQ ID NO:35, said vhCDR2 has SEQ ID NO:36, and vhCDR3 has SEQ ID NO:37.

72. A composition according to claim 59 wherein said vhCDR1 has SEQ ID NO:38, said vhCDR2 has SEQ ID NO:39, and vhCDR3 has SEQ ID NO:40.

73. A composition according to claim 59 wherein said vhCDR1 has SEQ ID NO:41, said vhCDR2 has SEQ ID NO:42, and vhCDR3 has SEQ ID NO:43.

74. A composition according to claim 59 wherein said vhCDR1 has SEQ ID NO44:, said vhCDR2 has SEQ ID NO45:, and vhCDR3 has SEQ ID NO:46.

75. A composition according to claim 59 wherein said vhCDR1 has SEQ ID NO:47, said vhCDR2 has SEQ ID NO:48, and vhCDR3 has SEQ ID NO:49.

76. A composition according to claim 59 wherein said vhCDR1 has SEQ ID NO:50, said vhCDR2 has SEQ ID NO:51, and vhCDR3 has SEQ ID NO:52.

77. A composition according to claim 59 wherein said vhCDR1 has SEQ ID NO:53, said vhCDR2 has SEQ ID NO:54, and vhCDR3 has SEQ ID NO:55.

78. A composition according to claim 59 wherein said vhCDR1 has SEQ ID NO:56, said vhCDR2 has SEQ ID NO:57, and vhCDR3 has SEQ ID NO:58.

79. A composition according to claim 59 wherein said vhCDR1 has SEQ ID NO:59, said vhCDR2 has SEQ ID NO:60, and vhCDR3 has SEQ ID NO:61.

80. A composition according to claim 59 wherein said vhCDR1 has SEQ ID N062 said vhCDR2 has SEQ ID NO:63, and vhCDR3 has SEQ ID NO:64.

81. A composition according to claim 59 wherein said vhCDR1 has SEQ ID NO:65, said vhCDR2 has SEQ ID NO:66, and vhCDR3 has SEQ ID NO:67.

82. A composition according to claim 59 wherein said vhCDR1 has SEQ ID NO:68, said vhCDR2 has SEQ ID NO:69, and vhCDR3 has SEQ ID NO70.

83. A composition according to claim 59 wherein said vhCDR1 has SEQ ID NO:71, said vhCDR2 has SEQ ID NO:72, and vhCDR3 has SEQ ID NO:73.

84. A composition according to claim 59 wherein said vhCDR1 has SEQ ID NO:74, said vhCDR2 has SEQ ID NO:75, and vhCDR3 has SEQ ID NO:76.

85. A composition according to any of claims 61 to 83 wherein the framework sequences of said sdABD have SEQ ID NO:1, SEQ ID NO:2, SEQ ID NO:3 and SEQ ID NO:4.

86. A composition according to any of claims 59 to 83 wherein said composition further comprises an half-life extension domain covalently attached to said sdABD using a domain linker.

87. A composition according to claim 84 wherein said half-life extension domain is selected from the group consisting of an anti-human serum albumin (HSA) sdABD and all or part of HSA.

88. A nucleic acid encoding the composition of any of claims 1 to 29, 30 to 58 and 59 to 87.

89. An expression vector comprising the nucleic acid of claim 88.

90. A host cell comprising the expression vector of claim 60.

91. A method of making a composition according to any of claims 1 to 29, 30 to 58 and 59 to 87 comprising culturing the host cell of claim 90 under conditions whereby said composition is expressed and recovering said composition.

92. A method of treating or preventing an infection by SARS-CoV2 virus comprising administering to a human the composition of any of claims 1 to 29, 30 to 58 and 59 to 87, thereby treating or preventing said infection.

93. A method of neutralizing the SARS-CoV2 virus comprising administering to a human the composition of any of claims 1 to 29, 30 to 58 and 59 to 87, thereby neutralizing said virus.

94. A method according to claim 92 or 93 wherein said administration comprises intraveneous administration.

95. A method according to claim 92 or 93 wherein said administration comprises nasal administration.

96. A method according to claim 92 or 93 wherein said administration comprises inhalation.

97. A method according to claim 96 wherein said inhalation is achieved using a nebulizer.

98. A lyophilized composition comprising a MASC protein of any of claims 1 to 29, 30 to 58 and 59 to 87.

99. A composition comprising an antigen binding domain (ABD) that that binds to the trimeric Spike protein, each monomer of the Spike protein having SEQ ID NO: 300 or the sequence described in FIG. 17A, wherein said ABD binds to a first epitope of a first monomer and binds to a second epitope of a second monomer.

100. A composition according to claim 99 where said first epitope comprises residues 446, 447, 449, 453, 455, 456, 483-486, 489-490, 493-496, 498, 501, and 505 within the ACE2 binding region of the SC2 spike RBD and said second epitope comprises residues 342, 343, 367, 371-375, 404, 436-441.

101. A composition comprising an ABD that binds to the trimeric Spike protein, each monomer of the Spike protein having SEQ ID NO: 300 or the sequence described in FIG. 17A, wherein said binding results in the RBD of said Spike protein being locked into the non-extended position.
